(19) United States
(12) Patent Application Publication
(10) Pub. No.: US 2024/0226187 A9
(43) Pub. Date: Jul. 11, 2024

(54) Compositions and Methods for Delivering a Bacterial Metabolite to a Subject

(71) Applicant: Finch Therapeutics Holdings LLC, Somerville, MA (US)

(72) Inventors: Mark SMITH, Cambridge, MA (US); Sonia TIMBERLAKE, Brookline, MA (US); Zain KASSAM, Medford, MA (US); Anh-Thu Elaine VO, Brookline, MA (US); John HENSKE, Somerville, MA (US); Ylaine GERARDIN, Cambridge, MA (US); Rotem Gura SADOVSKY, Somerville, MA (US)

(73) Assignee: Finch Therapeutics Holdings LLC, Somerville, MA (US)

(21) Appl. No.: 17/769,464

(22) PCT Filed: Oct. 19, 2020
(86) PCT No.: PCT/US2020/056369

§ 371 Date: Apr. 15, 2022

Related U.S. Application Data
(60) Provisional application No. 62/916,847, filed on Oct. 18, 2019.
(60) Provisional application No. 62/979,891, filed on Feb. 21, 2020.
(60) Provisional application No. 63/032,108, filed on May 29, 2020.

(51) Int. Cl. A61K 35/74; A61K 9/19; A61P 1/04

(52) U.S. Cl. CPC A61K 35/74; A61K 9/19; A61P 1/04

(57) ABSTRACT

The present disclosure relates to compositions comprising preparations of uncultured fecal bacteria, methods of manufacturing the compositions, and methods of using the compositions to treat a disorder related to a gut dysbiosis.

### RELATED APPLICATIONS

This application claims priority to U.S. Provisional Patent Application No. 62/916,847, filed Oct. 18, 2019; U.S. Provisional Patent Application No. 62/979,891, filed Feb. 21, 2020; and U.S. Provisional Patent Application No. 63/032,108, filed May 29, 2020, the contents of which are incorporated herein by reference in their entireties.

### BACKGROUND

Implantation or administration of human colonic microbiota into the bowel of a sick patient is called Fecal Microbiota Transplantation (FMT), also commonly known as fecal bacteriotherapy. FMT is believed to repopulate the gut with a diverse array of microbes that control key pathogens by creating an ecological environment inimical to their proliferation and survival. It represents a therapeutic protocol that allows a fast reconstitution of a normal compositional and functional gut microbial community.

FMT has been used to treat Clostridium difficile infection (CDI). FMT has also been suggested in treating other gut infective agents such as E. coli and Vancomycin resistant Enterococci (VRE). It entails infusions through a colonoscope, an enema or via a nasojejunal tube of human microbiota either in the form of homogenized stool, or cultured stool components such as Clostridia, to implant in the colon and thereby displace or eradicate pathogenic bacteria, e.g., C. difficile. Fecal bacteriotherapy has also been successful in treating conditions having a neurological component, such as ASD, Parkinson's Disease, and Multiple Sclerosis and Chronic Fatigue Syndrome.

Compositions for performing FMT typically incorporate the fecal microbiota of stool collected from healthy human donors. However, the microbial content of stool is not uniform across donors, or even longitudinally across different samples collected from the same donor. As a result, different doses of an FMT composition can vary in microbial constitution, for example in the presence or absence of a specific strain of bacteria or in the relative abundance of a bacterial strain.

An inability to regulate the identity and/or relative abundances of strains of bacteria in an FMT formulation may result in reduced and/or variable efficacy when administered to patients, if a particular bacterial strain or group of strains is associated with or needed for the treatment of a condition, for example by inducing a therapeutic response in a patient's cells.

### SUMMARY

In an aspect, the present disclosure provides a pharmaceutical composition comprising a preparation of fecal bacteria from a stool of a healthy human donor, wherein the fecal bacteria are selected to produce at least one short chain fatty acid (SCFA), wherein the preparation of fecal bacteria comprises uncultured bacteria, and wherein the preparation of fecal bacteria comprises lyophilized bacteria.

In another aspect, the present disclosure provides a method of delivering at least one SCFA to a subject in need thereof comprising administering to the subject a pharmaceutical composition disclosed here.

In an aspect, the present disclosure provides a method of treating a gut dysbiosis in a subject in need thereof comprising administering to the subject a pharmaceutical composition disclosed here.

In another aspect, the present disclosure provides a method of delivering a short chain fatty acid (SCFA) to an intestine of a subject in need thereof, the method comprising administering to the subject a pharmaceutical composition comprising a preparation of uncultured fecal bacteria derived from a stool of a healthy human donor, wherein (1) the uncultured fecal bacteria are selected to produce the SCFA at or above a threshold level, (2) fecal bacteria in a stool of the healthy human donor produce the SCFA at or above a threshold level, or (3) the healthy human donor produces a stool comprising the SCFA at or above a threshold level.

In an aspect, the present disclosure provides a method comprising determining a level of at least one SCFA produced by fecal bacteria of a subject; and administering to the subject a pharmaceutical composition based on determining the level of the at least one SCFA to be below a threshold level, wherein the pharmaceutical composition comprises a preparation of uncultured fecal bacteria of stool of a healthy human donor.

In another aspect, the present disclosure provides a method comprising: determining a level of a metabolite produced by fecal bacterial cells to be at or above a threshold level, wherein the fecal bacterial cells are from a stool of a healthy human donor; and extracting fecal bacteria from a stool of the donor based on determining the level of the metabolite to be at or above the threshold level to produce a preparation of uncultured fecal bacteria.

In an aspect, the present disclosure provides a method comprising: determining that a level of a short-chain fatty acid (SCFA) produced by fecal bacterial cells is at or above a threshold level, wherein the fecal bacterial cells are from a stool of a healthy human donor; selecting fecal bacteria of the donor based on determining that the level of the SCFA is at or above the threshold level, wherein the selected fecal bacteria are uncultured; and mixing the selected fecal bacteria with a cryoprotectant to produce a preparation of uncultured fecal bacteria.

In another aspect, the present disclosure provides a method comprising: determining that a relative abundance of one or more short chain fatty acid (SCFA)-producing bacterial strains is at or above a threshold level in stool of a healthy human donor; selecting fecal bacteria of the donor based on determining that the relative abundance of the one or more SCFA-producing bacterial strains is at or above the threshold level, wherein the selected fecal bacteria are uncultured; and mixing the selected fecal bacteria with a cryoprotectant to produce a preparation of uncultured fecal bacteria.

In an aspect, the present disclosure provides a method comprising: determining from a first stool of a donor that an SCFA in the first stool is at or above a threshold level; determining from a second stool of the donor that the SCFA in the second stool is at or above the threshold level; and extracting fecal bacteria from a stool of the donor to produce a preparation of uncultured fecal bacteria based on determining that the SCFA in the first and second stools is at or above a threshold level.

In another aspect, the present disclosure provides a method comprising: determining in a functional assay that fecal bacterial cells from a first stool of a donor produce an SCFA at or above a threshold level; determining in the functional assay that fecal bacterial cells from a second stool of the donor produce the SCFA at or above the threshold level; and extracting fecal bacteria from a stool of the donor to produce a preparation of uncultured fecal bacteria based on determining that the fecal bacterial cells from the first and second stools produce the SCFA at or above the threshold level in the functional assay.

In an aspect, the present disclosure provides a method comprising: determining from a first stool of a donor that a relative abundance of one or more SCFA-producing bacteria is at or above a threshold level; determining from a second stool of the donor that a relative abundance of one or more SCFA-producing bacteria is at or above the threshold level; and extracting fecal bacteria from a stool of the donor to produce a preparation of uncultured fecal bacteria based on determining that the relative abundance of one or more SCFA-producing bacteria is at or above the threshold level in the first and second stools.

In another aspect, the present disclosure provides a pharmaceutical composition comprising a preparation of fecal bacteria from stool of a healthy human donor, wherein the fecal bacteria produce an amount of butyrate at or above a threshold level of butyrate when incubated with a substrate in a functional assay, wherein the preparation of fecal bacteria comprises uncultured bacteria, and wherein the threshold level of butyrate is at least 40 mM.

In an aspect, the present disclosure provides a pharmaceutical composition comprising a preparation of fecal bacteria from stool of a healthy human donor, wherein the fecal bacteria produce an amount of acetate at or above a threshold level of acetate when incubated with a substrate in a functional assay, wherein the preparation of fecal bacteria comprises uncultured bacteria, and wherein the threshold level of acetate is at least 60 mM.

In another aspect, the present disclosure provides a pharmaceutical composition comprising a preparation of fecal bacteria from stool of a healthy human donor, wherein the fecal bacteria produce an amount of propionate at or above a threshold level of propionate when incubated with a substrate in a functional assay, wherein the preparation of fecal bacteria comprises uncultured bacteria, and wherein the threshold level of propionate is at least 10 mM.

In an aspect, the present disclosure provides a pharmaceutical composition comprising a preparation of fecal bacteria from stool of a healthy human donor, wherein the preparation of fecal bacteria comprises short-chain fatty acid (SCFA)-producing bacterial strains, wherein the SCFA-producing bacterial strains represent at least 40% of the total number of bacterial strains in the preparation of fecal bacteria, and wherein the preparation of fecal bacteria comprises uncultured bacteria.

In another aspect, the present disclosure provides a method comprising extracting fecal bacteria from a stool of a healthy human donor to produce a preparation of uncultured fecal bacteria, wherein the healthy human donor is pre-selected for a fecal metabolite at or above a threshold level in one or more, two or more, three or more, four or more, five or more, six or more, seven or more, or ten or more stool samples from the donor.

In an aspect, the present disclosure provides a method comprising extracting fecal bacteria from a stool of a healthy human donor to produce a preparation of uncultured fecal bacteria, wherein one or more, two or more, three or more, four or more, five or more, six or more, seven or more, or ten or more stool samples of the healthy human donor comprise a fecal metabolite at or above a threshold level.

In another aspect, the present disclosure provides a method comprising extracting fecal bacteria from a stool of a healthy human donor to produce a preparation of uncultured fecal bacteria, wherein one or more, two or more, three or more, four or more, five or more, six or more, seven or more, or ten or more stool samples of the healthy human donor are capable of producing a fecal metabolite at or above a threshold level.

In an aspect, the present disclosure provides a method comprising administering a pharmaceutical composition to a subject in need thereof, wherein the pharmaceutical composition comprises a preparation of uncultured fecal bacteria from a stool of a healthy human donor, wherein the subject is pre-selected for at least one SCFA at or below a threshold level in one or more, two or more, three or more, four or more, five or more, six or more, seven or more, or ten or more stool samples from the subject.

In another aspect, the present disclosure provides a method comprising administering a pharmaceutical composition to a subject in need thereof, wherein the pharmaceutical composition comprises a preparation of uncultured fecal bacteria from a stool of a healthy human donor, wherein one or more, two or more, three or more, four or more, five or more, six or more, seven or more, or ten or more stool samples of the subject comprise at least one SCFA at or below a threshold level.

In an aspect, the present disclosure provides a method comprising administering a pharmaceutical composition to a subject in need thereof, wherein the pharmaceutical composition comprises a preparation of uncultured fecal bacteria from a stool of a healthy human donor, wherein one or more, two or more, three or more, four or more, five or more, six or more, seven or more, or ten or more stool samples of the subject are capable of producing at least one SCFA at or below a threshold level.

In another aspect, the present disclosure provides a method comprising administering a pharmaceutical composition to a subject in need thereof, wherein the pharmaceutical composition comprises a preparation of uncultured fecal bacteria from a stool of a healthy human donor, wherein the subject is pre-selected for at least one SCFA at or above a threshold level in one or more, two or more, three or more, four or more, five or more, six or more, seven or more, or ten or more stool samples from the subject.

In an aspect, the present disclosure provides a method comprising administering a pharmaceutical composition to a subject in need thereof, wherein the pharmaceutical composition comprises a preparation of uncultured fecal bacteria from a stool of a healthy human donor, wherein one or more, two or more, three or more, four or more, five or more, six or more, seven or more, or ten or more stool samples of the subject comprise at least one SCFA at or above a threshold level.

In another aspect, the present disclosure provides a method comprising administering a pharmaceutical composition to a subject in need thereof, wherein the pharmaceutical composition comprises a preparation of uncultured fecal bacteria from a stool of a healthy human donor, wherein one or more, two or more, three or more, four or more, five or more, six or more, seven or more, or ten or more stool samples of the subject are capable of producing at least one SCFA at or above a threshold level.

In another aspect, the present disclosure provides a pharmaceutical composition comprising a preparation of fecal bacteria from a stool of a healthy human donor, wherein (i) the fecal bacteria are selected to produce at least one bile acid, (ii) the healthy human donor is selected for its level of at least one bile acid transforming bacterial strain, or (iii) the healthy human donor has a predetermined level of at least one bile acid or at least one bile acid transforming bacterial strain, wherein the fecal bacteria comprises uncultured bacteria, and wherein the preparation of fecal bacteria comprises lyophilized bacteria.

In yet another aspect, the present disclosure provides a method of selecting a stool of a donor for producing a preparation of uncultured fecal bacteria comprising determining the relative abundance of secondary bile acid in a first stool of an individual is at or above a threshold level, determining that a relative abundance of the secondary bile acid in a second stool of a donor is at or above a threshold level, and selecting the individual as the stool donor based on determining that the relative abundance of the secondary bile acid in the first and second stools is at or above the threshold level.

In an aspect, the present disclosure provides a method of selecting a stool of a donor for producing a preparation of uncultured fecal bacteria comprising determining the relative abundance of secondary bile acid in a stool of an individual and selecting the individual as a stool donor if the relative abundance of the secondary bile acid is at or above a threshold level.

In another aspect, the present disclosure provides a method of selecting a stool of a donor for producing a preparation of fecal bacteria comprising determining the relative abundance of bacteria capable of transforming primary bile acids to secondary bile acids and selecting the stool if the relative abundance of the secondary bile acids is at or above the threshold level.

In another aspect, the present disclosure provides a method of selecting a desired donor by screening potential donors' fecal microbial gene content associated with bile acid transforming strains.

In another aspect, the present disclosure provides a method of treating an irritable bowel disease (IBD) patient in need thereof comprising administering a preparation of fecal bacteria replete with bile acid transforming strains, wherein the patient in need thereof has a deficient bile acid metabolism.

In a further aspect, the present disclosure provides a method of treating an irritable bowel disease (IBD) patient in need thereof comprising administering a preparation of fecal bacteria with a relative abundance of secondary bile acids, wherein the patient in need thereof has a deficient bile acid metabolism and the relative abundance of secondary bile acid is at or above a threshold level.

In another aspect, the present disclosure provides a method of treating an irritable bowel disease (IBD) in a patient in need thereof having a deficient bile acid metabolism comprising administering to the patient a preparation of fecal bacteria derived from a donor having a quantity of secondary bile acids above a threshold and bile acid transforming strains.

In another aspect, the present disclosure provides a method of treating Crohn's disease or ulcerative colitis in a patient with deficient bile acid metabolism comprising treating the patient with donor materials replete with (i) a quantity of secondary bile acids and (ii) a quantity of bile acid transforming strains, wherein the quantity is at or above a predetermined threshold.

In a further aspect, the present disclosure provides a method of treating a Crohn's disease patient with deficient bile acid metabolism by increasing bile acid transforming strains in the patient comprising administering to the patient a preparation of fecal bacteria derived from a donor having a quantity of bile acid transforming strains at or above a threshold level, wherein the patient's bile acid metabolism is restored.

In yet another aspect, the present disclosure provides a method of treating an ulcerative colitis patient with deficient bile acid metabolism by increasing bile acid transforming strains in the patient comprising administering to the patient a preparation of fecal bacteria derived from a donor having a quantity of bile acid transforming strains at or above a threshold level, wherein the patient's bile acid metabolism is restored.

In another aspect, the present disclosure provides methods for delivering a bile acid transforming bacteria to an intestine of a subject in need thereof, the method comprising administering to the subject a pharmaceutical composition comprising a preparation of uncultured fecal bacteria derived from a stool of a healthy human donor, wherein: the uncultured fecal bacteria are selected to transform primary bile acid to secondary bile acid at or above a threshold level, fecal bacteria in a stool of the healthy human donor produce the secondary bile acid at or above a threshold level, or the healthy human donor produces a stool comprising the secondary bile acid at or above a threshold level.

In another aspect, the present disclosure also provides methods determining a level of at least one secondary bile acid produced by fecal bacteria of a subject comprising at least one bile acid transforming bacterial strain; and administering to the subject a pharmaceutical composition based on determining the level of the at least one secondary bile acid to be below a threshold level, wherein the pharmaceutical composition comprises a preparation of uncultured fecal bacteria of stool of a healthy human donor.

In another aspect, the present disclosure also provides method comprising: determining a level of a secondary bile acid transformed by fecal bacterial cells to be at or above a threshold level, wherein the fecal bacterial cells are from a stool of a healthy human donor; and extracting fecal bacteria from a stool of the donor based on determining the level of the secondary bile acid to be at or above the threshold level to produce a preparation of uncultured fecal bacteria.

In a further aspect, the present disclosure provides for methods comprising: determining that a level of secondary bile acids transformed by fecal bacterial cells is at or above a threshold level, wherein the fecal bacterial cells are from a stool of a healthy human donor; selecting fecal bacteria of the donor based on determining that the level of the secondary bile acid is at or above the threshold level, wherein the selected fecal bacteria are uncultured; and mixing the selected fecal bacteria with a cryoprotectant to produce a preparation of uncultured fecal bacteria.

In yet another aspect, the present disclosure provides for methods comprising: determining that a relative abundance of one or more bile acid transforming bacterial strains is at or above a threshold level in stool of a healthy human donor; selecting fecal bacteria of the donor based on determining that the relative abundance of the one or more bile acid transforming bacterial strains is at or above the threshold level, wherein the selected fecal bacteria are uncultured; and mixing the selected fecal bacteria with a cryoprotectant to produce a preparation of uncultured fecal bacteria.

In an even further aspect, the present disclosure provides for a pharmaceutical composition comprising a preparation of fecal bacteria from stool of a healthy human donor, wherein the fecal bacteria transform an amount of primary bile acid to secondary bile acid, wherein the secondary bile acid is at or above a threshold level of secondary bile acid when incubated with a substrate in a functional assay, wherein the preparation of fecal bacteria comprises uncultured bacteria, and wherein the threshold level of the secondary bile acid is at least 100 μM.

In an aspect, the present disclosure provides methods comprising extracting fecal bacteria from a stool of a healthy human donor to produce a preparation of uncultured fecal bacteria, wherein the healthy human donor is pre-selected for secondary bile acid at or above a threshold level in one or more, two or more, three or more, four or more, five or more, six or more, seven or more, or ten or more stool samples from the donor.

### BRIEF DESCRIPTION OF THE DRAWINGS

FIG. 1: Butyrate concentration (mM) produced by fecal bacteria after 12 hours of incubation with carbohydrate substrates in an ex vivo assay. For all FIG. 1 to FIG. 3, the x-axis represents the donor (G, S, E, P or U) of the stool used for the ex vivo assay. Each of donors G and U provided two samples (for G, collected on Aug. 29 and Sep. 20 2018; for U, collected on Aug. 29 and 30, 2018). “SPB” is the control sodium phosphate buffer (also used as a control with each donor sample). The y-axis represents mM of butyrate produced during the ex vivo assay.

FIG. 2: Acetate concentration (mM) produced by fecal bacteria after 12 hours of incubation with carbohydrate substrates in an ex vivo assay.

FIG. 3: Propionate concentration (mM) produced by fecal bacteria after 12 hours of incubation with carbohydrate substrates in an ex vivo assay.

FIG. 4A: Butyrate concentration (μg/g) determined from stool of healthy human donors.

FIG. 4B: Change in butyrate concentration (μg/g) induced in stool of IBD patients (diagnosed with Crohn's Disease (CD) or Ulcerative Colitis (UC)) after receiving FMTs containing fecal bacteria from each of the donors in FIG. 4A.

FIG. 4C: Change in butyrate concentration (μg/g) in two donors over time.

FIG. 5: Correlation of differences between donor butyrate concentration and patient baseline butyrate concentration with the differences between Post-FMT and baseline butyrate concentration in Crohn's Disease (CD) or Ulcerative Colitis (UC) patients.

FIG. 6: Correlation of Faecalibacterium abundance (black lines) with butyrate concentration (grey lines) in patients post-FMT treatment. Spearman correlations are indicated in the subplot titles. The dashed lines indicate average donor values of both Faecalibacterim (black dashed) and butyrate (grey dashed).

FIG. 7: Correlative analysis of Firmicutes, Proteobacteria, and Bacteroidetes abundance shift with butyrate concentration in both Ulcerative Colitis (UC) and Crohn's Disease (CD) patients at 12 weeks post-FMT treatment.

FIGS. 8A and 8B: Firmicutes, Proteobacteria, and Bacteroidetes abundance in UC (FIG. 8A) and CD (FIG. 8B) patients post-FMT treatment.

FIGS. 9A and 9B: Correlation of patient Partial Mayo index for UC (FIG. 9A) and Harvery-Bradshaw index for CD (FIG. 9B) with Proteobacteria abundance (correlation p-values for UC, p=0.150 and CD, p=0.502).

FIGS. 10A and 10B: Analysis of the similarity of UC (FIG. 10A) and CD (FIG. 10B) patients to donor in the weeks post-FMT treatment.

FIG. 11: Analysis of butyrate concentration changes in patients who become more similar to their donors.

FIG. 12: The relative abundance of butyrate producers in stool of 115 stool donors.

FIG. 13: Analysis showing efficacy of a donor selection strategy to account for natural variation in the presence and abundance of bacterial taxa between individual donors' fecal microbiota.

### DETAILED DESCRIPTION

Unless defined otherwise, all technical and scientific terms used herein have the same meaning as commonly understood by one of ordinary skill in the art to which this disclosure belongs.

All publications, patents, and patent applications mentioned in this specification are herein incorporated by reference to the same extent as if each individual publication, patent, or patent application was specifically and individually indicated to be incorporated by reference.

As used herein and in the appended claims, the singular forms “a,” “an,” and “the” include plural referents unless the context clearly dictates otherwise. By way of example, “an element” means at least one element and can include more than one element.

As used herein, the term “substantially”, when used to modify a quality, generally allows certain degree of variation without that quality being lost. For example, in certain aspects such degree of variation can be less than 0.1%, about 0.1%, about 0.2%, about 0.3%, about 0.4%, about 0.5%, about 0.6%, about 0.7%, about 0.8%, about 0.9%, about 1%, between 1-2%, between 2-3%, between 3-4%, between 4-5%, or greater than 5% or 10%.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either both of those included limits are also included in the disclosure.

As used herein, the term “relative abundance” refers to relative representation of an organism of a particular kind (e.g., a bacterial strain, species, or genus) relative to all organisms of similar nature in a certain community (e.g., a preparation of uncultured fecal bacteria or a bacterial mixture). Relative abundance is calculated by dividing the number of an organism of a particular kind by the total number of all organisms of similar nature in a certain community. In an aspect, relative abundance is measured by qPCR comparing PCR products generated with 16S primers targeting specific bacterial strains of interest against PCR products generated with universal primers targeting all 16S sequences. See e.g., Chu, N., et al., “Profiling living bacteria informs preparation of fecal microbiota transplantations.” PLoS One 12(1): 1-16 (2017). In another aspect, the relative abundance is measured based on the number of sequence reads detected via high-throughput sequencing. Unless specified otherwise, a bacterial relative abundance mentioned herein is measured via high-throughput sequencing. In a further aspect, propidium monoazide (PMA) is used to differentiate between viable and dead fecal microbes as shown in Chu et al., PLoS One 12(1): 1-16 (2017).

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, a “subject” refers to any animal subject including humans, laboratory animals (e.g., primates, rats, mice), livestock (e.g., cows, sheep, goats, pigs, turkeys, chickens), and household pets (e.g., dogs, cats, rodents, etc.). Preferred subjects are human subjects. The human subject may be a pediatric, adult or a geriatric subject. In some aspects, the terms “patient” and “subject” are used interchangeably.

As used herein, a stool refers to a piece of solid matter or part thereof that is released by a mammal (e.g., human) from a bowel movement.

As used herein, a “microbiota” and “flora” refer to a community of microbes that live in or on a subject's body, both sustainably and transiently, including eukaryotes, archaea, bacteria, and viruses (including bacterial viruses (i.e., phage)). A “fecal microbiota” or “fecal microbiota preparation” refers to a community of microbes present in or prepared from a subject's feces. Typically a pharmaceutical composition described herein is prepared by incorporating such a fecal microbiota into the composition without culturing the fecal microbiota after its purification from a stool. Herein “uncultured fecal bacteria” or a “preparation of uncultured fecal bacteria” refer to a preparation comprising multiple non-pathogenic viable bacterial strains that have been harvested, extracted or purified from one or more stool samples, without culturing the strains (e.g. in culturing medium). Such a preparation of uncultured fecal bacteria can also be referred to as a collection of uncultured fecal bacteria or a population of uncultured fecal bacteria.

In some aspects, a preparation of uncultured fecal bacteria comprises non-selected fecal bacteria. Herein “non-selected fecal bacteria” refers to a collection of viable fecal bacterial strains (e.g., present in a fecal microbiota) extracted from one or more stool samples without subjecting the extracted bacteria to environmental conditions that intentionally select for a particular type, state or taxonomic category of bacteria (e.g., by deliberate removal of certain strains of bacteria, treatment of the bacteria with an agent such as ethanol or chloroform, or culturing). Such non-selected fecal bacteria can comprise bacterial strains in proportional content to corresponding bacterial strains in a fecal or intestinal microbiota of a normal healthy human. Steps taken to non-selectively extract fecal bacteria from a stool sample can include, for example, homogenization and filtering of the stool sample to separate the fecal bacterial strains from non-cellular stool material such as fiber and rough particulate matter, as well as, for example, eukaryotic host cells and viruses. Herein typically a non-selected fecal bacterial preparation can be prepared in either aerobic or anaerobic conditions, or a combination thereof. In certain aspects, a preparation of non-selected fecal bacteria comprises all or substantially all of the bacteria of a fecal microbiota of a stool sample. In certain aspects, a preparation of non-selected fecal bacteria comprises all or substantially all of the strains of a fecal microbiota of a stool sample. In certain aspects, a preparation of non-selected fecal bacteria comprises all or substantially all of the species of a fecal microbiota of a stool sample. In certain aspects, a preparation of non-selected fecal bacteria comprises all or substantially all of the genera of a fecal microbiota of a stool sample. In certain aspects, a preparation of non-selected fecal bacteria comprises all or substantially all of the phyla of a fecal microbiota of a stool sample. Therefore, such non-selective fecal microbiota can substantially resemble microbial constituents and the bacterial population structure found in such fecal sample.

In an aspect, a preparation of uncultured fecal bacteria comprises at least 2, 5, 10, 20, 30, 40, 50, 100, 200, 300, 400, 500, or 600 bacterial species or strains. In another aspect, a preparation of uncultured fecal bacteria comprises between 2 and 5, 5 and 10, 10 and 20, 20 and 30, 30 and 40, 40 and 50, 50 and 60, 60 and 100, 100 and 200, 200 and 300, 300 and 400, 400 and 500, or 500 and 600 bacterial species or strains.

In an aspect, a preparation of uncultured fecal bacteria and/or non-selected fecal bacteria does not comprise an antibiotic resistant population of bacteria.

In another aspect, the preparation of a composition comprising uncultured fecal bacteria can involve steps that select for a particular, type, state, or taxonomic category of bacteria (e.g., by deliberate removal of certain strains of bacteria, treatment of the population with a selective agent such as ethanol or chloroform, and/or screening of the bacteria for the ability to produce a metabolite at or above a threshold level).

Herein uncultured fecal bacteria are distinguished from a single, purified strain of bacteria such as a bacterial isolate. As used herein, “bacterial isolate” refers to an isolated group of substantially genetically identical bacterial cells generated by proliferation via binary fission from a single predecessor bacterial cell (e.g., by culturing the bacteria). Typically, a bacterial isolate is originally isolated as a single cell or genetically pure group of cells, for example, as a single colony on solid culture media or via serial dilutions in liquid culture, and thereafter archived (e.g. as a frozen stock) to provide a consistent and stable source for the isolate. Once isolated, in some aspects, a bacterial isolate can be grown as a pure culture of cells; in other aspects, multiple bacterial isolates can be grown simultaneously in the same vessel as a mixed culture. The term “substantially genetically identical” refers to the very high (e.g. >99.9%) genetic identity shared by different cells in uncontaminated pure compositions of bacterial isolates, owing to their proliferation from a common predecessor, but accounts for minor genetic dissimilarity between cells due to accumulations of relatively rare mutations. Generally, a bacterial isolate is synonymous with a pure culture of bacterial cells. Typically, herein a bacterial isolate consists of non-pathogenic bacteria. In an aspect, a bacterial isolate can be a probiotic, or an ingredient in a probiotic.

As used herein, the term “bacterial cocktail”, sometimes called a “bacterial consortium” or “synthetic bacterial mixture”, refers to an engineered mixture of bacteria comprising a defined consortium of multiple bacterial isolates. The term “defined consortium of multiple bacterial isolates” means that the bacterial cocktail contains two or more bacterial isolates, and that the identity of each bacterial isolate in the cocktail is known, and thus the cocktail can be consistently produced (e.g. by combining isolated bacterial strains) to have a stable composition and properties across separate batches. Herein “identity” of a bacterial isolate can refer to any characteristic of the isolate that uniquely identifies the isolate as different from one or more other bacterial isolates or bacterial strains. Examples of identifying characteristics of a bacterial isolate include nucleotide sequences such as a 16S rRNA sequence, the sequence of one or more coding or non-coding regions of a nucleic acid, and entire genome sequences, levels of gene expression, physiological or metabolic traits, or anatomical traits such as staining pattern or cell wall characteristics.

As used herein, “bacterial mixture” refers to an engineered composition comprising viable bacterial cells. In some aspects, a bacterial mixture comprises one or more non-pathogenic bacterial isolates (e.g., comprising an SCFA-producing bacterial strain). In some aspects, a bacterial mixture comprises a preparation of uncultured fecal bacteria. In some aspects, a bacterial mixture comprises both of one or more non-pathogenic bacterial isolates and a preparation of uncultured fecal bacteria.

As used herein, “SCFA-producing bacterial strain”, which may also be referred to as an “SCFA-producing bacterial isolate” refers to a bacterial strain capable of producing and/or secreting an SCFA. In one aspect, an SCFA-producing bacterial strain is in the form of a bacterial isolate. In another aspect, an SCFA-producing bacterial strain is a part of or a component of a preparation of uncultured fecal bacteria. In aspects, an SCFA-producing bacterial strain can be a member of Firmicutes, Bacteroidetes, Eubacterium sp., Clostridium sp., Faecalibacterium sp., Roseburia sp., Butyrivibrio sp., Anaerostipes sp., Coprococcus sp., Subdoligranulum sp., Anaerotruncus sp., Ruminococcus sp., Eubacterium rectale, Roseburia intestinalis, Roseburia faecis, Roseburia hominis, Roseburia inulinivorans, Roseburia cecicola, Butyrivibrio fibrisolvens, Eubacterium ramulus, Eubacterium hallii, Eubacterium ruminantium, Eubacterium cylindroides, Eubacterium oxidoreducens, Coprococcus catus, Coprococcus eutactus, Coprococcus comes, Faecalibacterium prausnitzii, Subdoligranulum variabile, Anaerotruncus colihominis, Clostridium nexile, Clostridium hathewayi, Clostridium indolis, Clostridium leptum, Ruminococcus gnavus, Ruminococcus obeum, or Anaerostipes caccae. In an aspect, an SCFA-producing bacterial strain can be a member of Clostridium cluster IV or XIVa.

As used herein, “therapeutically effective amount,” “effective amount” or “pharmaceutically active dose” refers to an amount of a composition which is effective in treating the named disease, disorder, condition, or symptom.

As used herein, “isolated” or “purified” refers to a bacterium or other entity or substance that has been (1) separated from at least some of the components with which it was associated when initially produced (whether it was initially produced in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

As used herein, the terms “non-pathogenic” in reference to a bacterium or any other organism or entity includes any such organism or entity that is not capable of causing or affecting a disease, disorder or condition of a host organism containing the organism or entity.

As used herein, “spore” or a population of “spores” includes bacteria (or other single-celled organisms) that are generally viable, more resistant to environmental influences such as heat and bacteriocidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, “colony forming units” (CFUs) refers to an estimate of the number of viable microorganism cells in a given sample. The number of CFUs can be assessed by counting the number of colonies on an agar plate as in standard methods for determining the number of viable bacterial cells in a sample.

As used herein, “viable” means possessing the ability to multiply. The viability of bacterial populations can be monitored as a function of the membrane integrity of the cell. Cells with a compromised membrane are considered to be dead or dying, whereas cells with an intact membrane are considered live. For example, SYTO 9 and propidium iodide are used to stain and differentiate live and dead bacteria. See Stocks, Cytometry A. 2004 October; 61(2):189-95. Cell viability can also be evaluated via molecular viability analyses, e.g., a PCR-based approach, which can differentiate nucleic acids associated with viable cells from those associated with inactivated cells. See Cangelosi and Mescheke, Appl Environ Microbiol. 2014 October; 80(19): 5884-5891.

As used herein, “Shannon Diversity Index” refers to a diversity index that accounts for abundance and evenness of species present in a given community using the formula H=−Σᵢ₌₁^{R}pᵢ ln pᵢ, where H is Shannon Diversity Index, R is the total number of species in the community, and pᵢ is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117 pp.

As used herein, “antibiotic” refers to a substance that is used to treat and/or prevent bacterial infection by killing bacteria, inhibiting the growth of bacteria, or reducing the viability of bacteria.

As used herein, “adverse events (AEs)” refers to any dose that results in procedure- or microbiota-related signs or symptoms. As used herein, “serious adverse events (SAEs)” refers to any medical occurrence that at any dose: results in death or is life-threatening. As used herein “life-threatening” refers to an event in which the patient is at risk of death at the time of the event. Adverse events are graded according to a scale used by one of ordinary skill in the art (e.g., National Cancer Institute (NCI) Common Terminology Criteria for Adverse Events (CTCAE)).

Described herein are pharmaceutical compositions comprising bacteria, methods of using the pharmaceutical compositions to treat or prevent a disorder, and methods of manufacturing the pharmaceutical compositions.

In aspects of the present disclosure, a pharmaceutical composition comprises a preparation of uncultured fecal bacteria selected to produce, release or secrete a metabolic product when administered to a patient. For example, in an aspect uncultured fecal bacteria (e.g., in the form of a fecal microbiota) extracted from the stool of a donor can be subjected to a screen, assay or test to determine the ability or potential or potential of the bacteria to produce a particular metabolite of interest (e.g., SCFA or secondary bile acid), and the fecal bacteria can then be selected for inclusion in a pharmaceutical composition based on the results of the screen, assay or test. In another aspect, a donor's raw stool or a sample of a raw stool (i.e., including fecal bacteria and non-floral material such as fiber) can be subjected to a screen, assay or test to determine the presence, absence and/or quantity of a particular metabolite of interest, and the fecal bacteria in the raw stool (i.e., which have produced the metabolite) can then be selected for inclusion in a pharmaceutical composition based on the results of the screen, assay or test.

In an aspect, a pharmaceutical composition comprises a preparation of uncultured fecal bacteria selected for inclusion in the composition on the basis of an ability of the bacteria to transform primary bile acid to secondary bile acid, for example at a level that is greater than a threshold level. In an aspect, “bile acid transforming bacteria” in reference to bacteria, bacterial taxa or bacterial isolates refers to one or more bacterial strains capable of transforming primary bile acid to secondary bile acid. Without wishing to be bound by theory, primary bile acids are synthetized in the liver from cholesterol in a process that constitutes the major pathway for cholesterol degradation. After synthesis, bile acid is stored in the gallbladder and later secreted into the duodenum. Bile is important for emulsification of dietary lipids, digestion and absorption of fatty acids, cholesterol, fat-soluble vitamins, and other hydrophobic diet components. A majority of secreted bile acids are reabsorbed in the small intestine. Bile acids that are not absorbed in the small intestine migrate to the large intestine where they are normally metabolized by the microbiota, before being reabsorbed from the colon. Fiamoncini, J., The Cross Talk Between Bile Acids and intestinal Microbiota, Microbiome and Metabolome in Diagnosis, Therapy, and other Strategic Applications. (2019). Deoxycholic acid, lithocholic acid, and ursodeoxycholic acid are the most common secondary bile acids formed through bacterial metabolism of primary bile acid in the human intestines.

In an aspect of the present disclosure primary bile acid is selected from the group consisting of cholic acid (CA), chenodeoxycholic acid (CDCA), glycocholic acid (GCA), taurocholic acid (TCA), glycochenodeoxycholic acid (GCDCA), taurochenodeoxycholic acid (TCDCA) and a combination thereof. In an aspect of the present disclosure provides a pharmaceutical composition comprising less than 200 μM. In another aspect, the pharmaceutical composition comprises less than 150 μM, 100 μM, 50 μM, 25 μM, 20 μM, 15 μM, 10 μM, or 5 μM.

In another aspect of the present disclosure secondary bile acid is selected from the group consisting of deoxycholic acid (DCA), isodeoxycholic acid, glycodeoxycholic acid (GDCA), taurodeoxycholic acid (TDCA), glycolithocholic acid (GLCA), taurolithocholic acid (TLCA), lithocholic acid (LCA), ursodeoxycholic acid (UDCA), isoursodeoxycholic acid, glycoursodeoxycholic acid (GUDCA), tauroursodeoxycholic acid (TUDCA) and a combination thereof. See Fiamoncini, Jarlei, Chapter 14—The Cross Talk Between Bile Acids and Intestinal Microbiota: Focus on Metabolic Diseases and Bariatric Surgery, Microbiome and Matabolome in Diagnosis, Therapy, and other Strategic Applications, pages 139-145 (2019) (incorporated herein by reference). In an aspect, the present disclosure provides a pharmaceutical composition comprising a preparation of uncultured fecal bacteria comprising at least one, at least two, at least three, at least four, or at least 5 secondary bile acids at or above a threshold level. In an aspect, the at least one secondary bile acid is deoxycholic acid (DCA) and the threshold level is 100 μM. In another aspect, the threshold level is 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, 200 μM, 300 μM, 350 μM, 400 μM, 450 μM, or 500 μM. In another aspect, the at least one secondary bile acid is deoxycholic acid (DCA) and the threshold level is between 5 μM and 20 μM, 5 μM and 50 μM, 5 μM and 100 μM, and 5 μM and 150 μM, 5 μM and 200 μM, 5 μM and 800 μM, 10 μM and 20 μM, 10 μM and 50 μM, 10 μM and 100 μM, 10 μM and 150 μM, 10 μM and 200 μM, 50 μM and 100 μM, 50 μM and 200 μM, 100 μM and 200 μM, 200 μM and 400 μM, 200 μM and 600 μM, 400 μM and 800 μM, or 200 μM and 800 μM. In another aspect, the at least one secondary bile acid is glycodeoxycholic acid (GDCA) and the threshold level is 100 μM. In another aspect, the threshold level is 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, or 200 μM. In another aspect, the at least one secondary bile acid is GDCA and the threshold level is between 5 μM and 20 μM, 5 μM and 50 μM, 5 μM and 100 μM, and 5 μM and 150 μM, 5 μM and 200 μM, 5 μM and 800 μM, 10 μM and 20 μM, 10 μM and 50 μM, 10 μM and 100 μM, 10 μM and 150 μM, 10 μM and 200 μM, 50 μM and 100 μM, 50 μM and 200 μM, 100 μM and 200 μM, or 200 μM and 800 μM. In another aspect, the at least one secondary bile acid is taurodeoxycholic acid (TDCA) and the threshold level is 100 μM. In another aspect, the threshold level is 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, or 200 μM. In another aspect, the at least one secondary bile acid is TDCA and the threshold level is between 5 μM and 20 μM, 5 μM and 50 μM, 5 μM and 100 μM, and 5 μM and 150 μM, 5 μM and 200 μM, 5 μM and 800 μM, 10 μM and 20 μM, 10 μM and 50 μM, 10 μM and 100 μM, 10 μM and 150 μM, 10 μM and 200 μM, 50 μM and 100 μM, 50 μM and 200 μM, 100 μM and 200 μM, or 200 μM and 800 μM. In yet another aspect, the at least one secondary bile acid is glycolithocholic acid (GLCA) and the threshold level is 100 μM. In another aspect, the threshold level is 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, or 200 μM. In another aspect, the at least one secondary bile acid is GLCA and the threshold level is between 5 μM and 20 μM, 5 μM and 50 μM, 5 μM and 100 μM, and 5 μM and 150 μM, 5 μM and 200 μM, 5 μM and 800 μM, 10 μM and 20 μM, 10 μM and 50 μM, 10 μM and 100 μM, 10 μM and 150 μM, 10 μM and 200 μM, 50 μM and 100 μM, 50 μM and 200 μM, 100 μM and 200 μM, or 200 μM and 800 μM. In another aspect, the at least one secondary bile acid is lithocholic acid (LCA) and the threshold level is 100 μM. In another aspect, the threshold level of LCA in raw stool used as a source for the preparation is at least 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, or 200 μM. In another aspect, the at least one secondary bile acid is LCA and the threshold level is between 5 μM and 20 μM, 5 μM and 50 μM, 5 μM and 100 μM, and 5 μM and 150 μM, 5 μM and 200 μM, 5 μM and 800 μM, 10 μM and 20 μM, 10 μM and 50 μM, 10 μM and 100 μM, 10 μM and 150 μM, 10 μM and 200 μM, 50 μM and 100 μM, 50 μM and 200 μM, 100 μM and 200 μM, or 200 μM and 800 μM. In another aspect, the at least one secondary bile acid is LCA and the threshold is at least 800 μM, at least 1000 μM, at least 1200 μM, at least 1400 μM, at least 1600 μM, at least 1800 μM, at least 2000 μM, or greater than 2000 μM in the raw stool of the donor. In another aspect, the at least one secondary bile acid is ursodeoxycholic acid (UDCA) and the threshold level is 100 μM. In another aspect, the threshold level is 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, or 200 μM. In another aspect, the at least one secondary bile acid is UDCA and the threshold level is between 5 μM and 20 μM, between 5 μM and 50 μM, between 5 μM and 100 μM, and between 5 μM and 150 μM, between 5 μM and 200 μM, between 5 μM and 800 μM, between 10 μM and 20 μM, between 10 μM and 50 μM, between 10 μM and 100 μM, between 10 μM and 150 μM, between 10 μM and 200 μM, between 50 μM and 100 μM, between 50 μM and 200 μM, between 100 μM and 200 μM, or between 200 μM and 800 μM. In yet another aspect, the at least one secondary bile acid is UDCA and the threshold level of UDCA in raw stool used as a source for the preparation is at least 0.25 μg/mg, at least 0.5 μg/mg, at least 0.75 μg/mg, at least 1.0 μg/mg, at least 1.25 μg/mg, at least 1.5 μg/mg, at least 1.75 μg/mg, at least 2.0 μg/mg, at least 2.25 μg/mg, at least 2.5 μg/mg, at least 2.75 μg/mg, or at least 3.0 μg/mg raw stool. In another aspect, the at least one secondary bile acid is UDCA and the threshold level of UDCA in raw stool used as a source for the preparation is greater than 3.0 μg/mg raw stool. In a further aspect, the at least one secondary bile acid is glycoursodeoxycholic acid (GUDCA) and the threshold level is 100 μM. In another aspect, the threshold level is 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, or 200 μM. In another aspect, the at least one secondary bile acid is deoxycholic acid (DCA) and the threshold level is between 5 μM and 20 μM, 5 μM and 50 μM, 5 μM and 100 μM, and 5 μM and 150 μM, 5 μM and 200 μM, 5 μM and 800 μM, 10 μM and 20 μM, 10 μM and 50 μM, 10 μM and 100 μM, 10 μM and 150 μM, 10 μM and 200 μM, 50 μM and 100 μM, 50 μM and 200 μM, 100 μM and 200 μM, or 200 μM and 800 μM. In an even further aspect, at least one secondary bile acid is tauroursodeoxycholic acid (TUDCA) and the threshold level is 100 μM. In another aspect, the threshold level is 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, or 200 μM. In another aspect, the at least one secondary bile acid is TUDCA and the threshold level is between 5 μM and 20 μM, 5 μM and 50 μM, 5 μMaud 100 μM, and 5 μM and 150 μM, 5 μM and 200 μM, 5 μM and 800 μM, 10 μM and 20 μM, 10 μM and 50 μM, 10 μM and 100 μM, 10 μM and 150 μM, 10 μM and 200 μM, 50 μM and 100 μM, 50 μM and 200 μM, 100 μM and 200 μM, or 200 μM and 800 μM.

In an aspect, a preparation of uncultured fecal bacteria incorporated into a pharmaceutical composition comprises one or more bile acid transforming bacterial taxa (e.g., phylum, class, order, family, genus, species or strain). In another example, a preparation of uncultured fecal bacteria incorporated into a pharmaceutical composition comprise one or more bacterial taxa (e.g., phylum, class, order, family, genus, species or strain) capable of transforming primary bile acid to secondary bile acids.

In another aspect of the present disclosure bile acid transforming bacterial strain is a member of a taxonomic group selected from the group consisting of Firmicutes, Bacteroidetes, Eubacterium sp., Clostridium sp., Faecalibacterium sp., Roseburia sp., Butyrivibrio sp., Anaerostipes sp., Coprococcus sp., Subdoligranulum sp., Anaerotruncus sp., Ruminococcus sp., Eubacterium rectale, Roseburia intestinalis, Roseburia faecis, Roseburia hominis, Roseburia inulinivorans, Roseburia cecicola, Butyrivibrio fibrisolvens, Eubacterium ramulus, Eubacterium hallii, Eubacterium ruminantium, Eubacterium cylindroides, Eubacterium oxidoreducens, Coprococcus catus, Coprococcus eutactus, Coprococcus comes, Faecalibacterium prausnitzii, Subdoligranulum variabile, Anaerotruncus colihominis, Clostridium nexile, Clostridium hathewayi, Clostridium indolis, Clostridium leptum, Ruminococcus gnavus, Ruminococcus obeum, and Anaerostipes caccae.

In another aspect of the present disclosure a bile acid transforming bacterial strain is a member of the families Lachnospiraceae, Ruminococcaceae, Bacteroidacaeae, Porphyromonadaceae, or Prevotellaceae.

In an aspect, a pharmaceutical composition comprises a preparation of uncultured fecal bacteria selected for inclusion in the composition on the basis of an ability of the bacteria to produce one or more short-chain fatty acids (SCFAs), for example at a level that is greater than a threshold level. As used herein, a “short-chain fatty acid” or “SCFA” refers to a fatty acid with an aliphatic tail of one to six carbon atoms. SCFAs can be produced by bacteria during bacterial metabolism, such as during fermentation of, for example, carbohydrates, proteins, peptides and glycoprotein precursors. Illustrative SCFAs include, but are not limited to, acetic acid (also known as acetate), butyric acid (also known as butyrate), caproic acid (also known as hexanoic acid or caproate), formic acid (also known as methanoic acid), heptanoic acid (also known as enanthic acid or heptanoate), isobutyric acid (also known as 2-methylpropanoic acid or isobutyrate), isocaproic acid (also known as 4-methylpentanoic acid or 4-methylvaleric acid or isocaproate), isovaleric acid (also known as 3-methylbutanoic acid or (3-methylbutyric acid or isovalerate), propionic acid (also known as propanoic acid or propionate), and valeric acid (also known as pentanoic acid or valerate). Without wishing to be bound by theory, SCFAs are thought to play an essential role in maintaining the health of colonic mucosa, and the presence of gut bacteria that produce SCFAs is associated with sustained clinical remission of certain gut dysbioses, such as inflammatory bowel disease (IBD). Accordingly, in some aspects, bacteria (e.g., uncultured fecal bacteria) incorporated into a pharmaceutical composition described herein are selected to produce one or more SCFAs. For example, uncultured fecal bacteria can produce one or more SCFAs in an intestine of a subject after the composition is administered to the subject (e.g., a subject having IBD). In another example, uncultured fecal bacteria can produce one or more SCFAs ex vivo, for example during performance of an assay capable of detecting and/or measuring a level of SCFA produced by fecal bacteria.

In an aspect, a pharmaceutical composition comprises bacteria (e.g., uncultured fecal bacteria) selected to produce one or more SCFAs at or above a particular threshold level. For example, a preparation of uncultured fecal bacteria incorporated into a pharmaceutical composition can produce or be selected to produce at least 0.5, at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 10.5, at least 11, at least 11.5, at least 12, at least 12.5, at least 13, at least 13.5, at least 14, at least 14.5, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, or greater than 200 mM of butyrate (for example in an intestine of a subject administered the composition or in an ex vivo assay measuring the level of butyrate produced by the bacteria in the presence of a substrate).

In another example, a preparation of uncultured fecal bacteria incorporated into a pharmaceutical composition can produce or be selected to produce at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, or greater than 200 mM of acetate (for example in an intestine of a subject administered the composition or in an ex vivo assay measuring the level of acetate produced by the bacteria in the presence of a substrate).

In another example, a preparation of uncultured fecal bacteria incorporated into a pharmaceutical composition can produce or be selected to produce at least 0.5, at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 10.5, at least 11, at least 11.5, at least 12, at least 12.5, at least 13, at least 13.5, at least 14, at least 14.5, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100, or greater than 100 mM of propionate (for example in an intestine of a subject administered the composition or in an ex vivo assay measuring the level of propionate produced by the bacteria in the presence of a substrate).

In another example, a preparation of uncultured fecal bacteria incorporated into a pharmaceutical composition can produce or be selected to produce at least 0.2, at least 0.4, at least 0.6, at least 0.8, at least 1, at least 1.2, at least 1.4, at least 1.6, at least 1.8, at least 2, at least 2.2, at least 2.4, at least 2.6, at least 2.8, at least 3, at least 3.2, at least 3.4, at least 3.6, at least 3.8, at least 4, at least 4.5, at least 5, at least 5.5, at least 6, at least 6.5, at least 7, at least 7.5, at least 8, at least 8.5, at least 9, at least 9.5 at least 10, or greater than 10 mM of caproate (for example in an intestine of a subject administered the composition or in an ex vivo assay measuring the level of caproate produced by the bacteria in the presence of a substrate).

In another example, a preparation of uncultured fecal bacteria incorporated into a pharmaceutical composition can produce or be selected to produce at least 0.2, at least 0.4, at least 0.6, at least 0.8, at least 1, at least 1.2, at least 1.4, at least 1.6, at least 1.8, at least 2, at least 2.2, at least 2.4, at least 2.6, at least 2.8, at least 3, at least 3.2, at least 3.4, at least 3.6, at least 3.8, at least 4, at least 4.5, at least 5, at least 5.5, at least 6, at least 6.5, at least 7, at least 7.5, at least 8, at least 8.5, at least 9, at least 9.5 at least 10, or greater than 10 mM of heptanoate (for example in an intestine of a subject administered the composition or in an ex vivo assay measuring the level of heptanoate produced by the bacteria in the presence of a substrate).

In another example, a preparation of uncultured fecal bacteria incorporated into a pharmaceutical composition can produce or be selected to produce at least 0.1, at least 0.2, at least 0.3, at least 0.4, at least 0.5, at least 0.6, at least 0.7, at least 0.8, at least 0.9, at least 1, at least 1.1, at least 1.2, at least 1.3, at least 1.4, at least 1.5, at least 1.6, at least 1.7, at least 1.8, at least 1.9, at least 2, at least 2.2, at least 2.4, at least 2.6, at least 2.8, at least 3, at least 3.2, at least 3.4, at least 3.6, at least 3.8, at least 4, at least 4.5, at least 5, at least 5.5, at least 6, at least 6.5, at least 7, at least 7.5, at least 8, at least 8.5, at least 9, at least 9.5 at least 10, or greater than 10 mM of isobutyrate (for example in an intestine of a subject administered the composition or in an ex vivo assay measuring the level of isobutyrate produced by the bacteria in the presence of a substrate).

In another example, a preparation of uncultured fecal bacteria incorporated into a pharmaceutical composition can produce or be selected to produce at least 0.1, at least 0.2, at least 0.3, at least 0.4, at least 0.5, at least 0.6, at least 0.7, at least 0.8, at least 0.9, at least 1, at least 1.1, at least 1.2, at least 1.3, at least 1.4, at least 1.5, at least 1.6, at least 1.7, at least 1.8, at least 1.9, at least 2, at least 2.2, at least 2.4, at least 2.6, at least 2.8, at least 3, at least 3.2, at least 3.4, at least 3.6, at least 3.8, at least 4, at least 4.5, at least 5, at least 5.5, at least 6, at least 6.5, at least 7, at least 7.5, at least 8, at least 8.5, at least 9, at least 9.5 at least 10, or greater than 10 mM of isocaproate (for example in an intestine of a subject administered the composition or in an ex vivo assay measuring the level of isocaproate produced by the bacteria in the presence of a substrate).

In another example, a preparation of uncultured fecal bacteria incorporated into a pharmaceutical composition can produce or be selected to produce at least 0.2, at least 0.4, at least 0.6, at least 0.8, at least 1, at least 1.2, at least 1.4, at least 1.6, at least 1.8, at least 2, at least 2.2, at least 2.4, at least 2.6, at least 2.8, at least 3, at least 3.2, at least 3.4, at least 3.6, at least 3.8, at least 4, at least 4.5, at least 5, at least 5.5, at least 6, at least 6.5, at least 7, at least 7.5, at least 8, at least 8.5, at least 9, at least 9.5 at least 10, or greater than 10 mM of isovalerate (for example in an intestine of a subject administered the composition or in an ex vivo assay measuring the level of isovalerate produced by the bacteria in the presence of a substrate).

In another example, a preparation of uncultured fecal bacteria incorporated into a pharmaceutical composition can produce or be selected to produce at least 0.2, at least 0.4, at least 0.6, at least 0.8, at least 1, at least 1.2, at least 1.4, at least 1.6, at least 1.8, at least 2, at least 2.2, at least 2.4, at least 2.6, at least 2.8, at least 3, at least 3.2, at least 3.4, at least 3.6, at least 3.8, at least 4, at least 4.5, at least 5, at least 5.5, at least 6, at least 6.5, at least 7, at least 7.5, at least 8, at least 8.5, at least 9, at least 9.5 at least 10, or greater than 10 mM of valerate (for example in an intestine of a subject administered the composition or in an ex vivo assay measuring the level of valerate produced by the bacteria in the presence of a substrate).

As described herein, uncultured fecal bacteria can be selected for inclusion in a pharmaceutical composition described herein (e.g., for delivering one or more SCFAs to an intestine of a subject administered the composition) on the basis of a level or quantity of an SCFA in a stool sample of a donor of the fecal bacteria. In another aspect, uncultured fecal bacteria can be selected for inclusion in a pharmaceutical composition described herein (e.g., for delivering one or more SCFAs to an intestine of a subject administered the composition) on the basis of a level or quantity of an SCFA produced by the fecal bacteria in an ex vivo assay. In another aspect, a preparation of uncultured fecal bacteria can be selected for inclusion in a pharmaceutical composition described herein (e.g., for delivering one or more SCFAs to an intestine of a subject administered the composition) on the basis of the relative abundance of one or more SCFA-producing bacterial strains in the preparation. In an aspect, a donor of stool used as starting material to prepare a preparation of uncultured fecal bacteria (e.g., selected on the basis of the relative abundance of one or more SCFA-producing bacterial strains) is administered a prebiotic prior to donating the stool. In another aspect, a donor of stool used as starting material to prepare a preparation of uncultured fecal bacteria (e.g., selected on the basis of the relative abundance of one or more SCFA-producing bacterial strains) is not administered a prebiotic prior to the donation.

In one aspect, a preparation of uncultured fecal bacteria comprises an entire or substantially complete fecal microbiota from stool of a donor. In one aspect, uncultured fecal bacteria comprise an isolated or purified population of live non-pathogenic fecal bacteria. In a further aspect, uncultured fecal bacteria comprise a substantially complete fecal microbiota preparation from a single donor. In another aspect, a pharmaceutical composition described herein comprises a bacterial mixture comprising one or more live, non-pathogenic, bacterial isolates and live, non-pathogenic, purified or extracted, uncultured fecal bacteria.

In an aspect, the preparation of uncultured fecal bacteria from stool of a donor involves a treatment selected from the group consisting of ethanol treatment, detergent treatment, heat treatment, irradiation, and sonication. In another aspect, the preparation of uncultured fecal bacteria from stool of a donor involves no treatment selected from the group consisting of ethanol treatment, detergent treatment, heat treatment, irradiation, and sonication. In one aspect, the preparation of uncultured fecal bacteria from stool of a donor involves a separation step selected from the group consisting of density gradients, filtration (e.g., sieves, nylon mesh), and chromatography. In another aspect, the preparation of uncultured fecal bacteria from stool of a donor involves no separation step selected from the group consisting of density gradients, filtration (e.g., sieves, nylon mesh), and chromatography. In another aspect, a preparation of uncultured fecal bacteria comprises an entire or substantially entire fecal microbiota from a stool sample of a donor. In another aspect, a pharmaceutical composition administered herein comprises a preparation of uncultured fecal bacteria substantially free of donor eukaryotic cells.

In an aspect, a pharmaceutical composition provided or administered herein comprises a preparation of uncultured fecal bacteria comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a pharmaceutical composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a pharmaceutical composition comprises a preparation of flora in proportional content that resembles a normal healthy human fecal flora.

In a further aspect, a pharmaceutical composition comprises fecal bacteria (e.g., uncultured fecal bacteria selected to produce one or more SCFAs at or above a threshold level) from at least 1, 2, 3, 4, 5, 6, 7, 8, 9, or 10 different families. In another aspect, a pharmaceutical composition comprises fecal bacteria from at least 11, 12, 13, 14, 15, 16, 17, 18, 19, or 20 different families. In yet another aspect, a pharmaceutical composition comprises fecal bacteria from at least 21, 22, 23, 24, 25, 26, 27, 28, 29, or 30 different families. In a further aspect, a pharmaceutical composition comprises fecal bacteria from at least 31, 32, 33, 34, 35, 36, 37, 38, 39, or 40 different families. In another aspect, a pharmaceutical composition comprises fecal bacteria from at least 41, 42, 43, 44, 45, 46, 47, 48, 49, or 50 different families. In another aspect, a pharmaceutical composition comprises fecal bacteria from between 1 and 10, between 10 and 20, between 20 and 30, between 30 and 40, between 40 and 50 different families. In an aspect, a pharmaceutical composition provided or administered herein comprises a preparation of uncultured fecal bacteria comprising no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. In another aspect, a pharmaceutical composition provided or administered herein comprises a preparation of fecal bacteria comprising no greater than 20%, 25%, 30%, 35%, 40%, 45%, 50%, 55%, 60%, 65%, 70%, 75%, 80%, 85%, 90%, or 95% weight non-living material/weight biological material. In another aspect, a pharmaceutical composition provided or administered herein comprises, consists of, or consists essentially of, particles of non-living stool material and/or particles of biological material of a fecal sample that passes through a sieve, a column, or a similar filtering device having a sieve, exclusion, or particle filter size of 2.0 mm, 1.0 mm, 0.5 mm, 0.33 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.002 mm. “Non-living stool material” refers to material present in stool when the stool is collected from a donor, and does not include an excipient, e.g., a pharmaceutically inactive substance, such as a cryoprotectant, added during processing of fecal material. “Biological material” refers to the living material in fecal material, and includes microbes including prokaryotic cells, such as bacteria and archaea (e.g., living prokaryotic cells and spores that can sporulate to become living prokaryotic cells), eukaryotic cells such as protozoa and fungi, and viruses. In one aspect, “biological material” refers to the living material, e.g., the microbes, eukaryotic cells, and viruses, which are present in the intestine (e.g., colon) of a normal healthy human. In an aspect, a pharmaceutical composition provided or administered herein comprises an extract of human stool, wherein the composition is substantially odorless. In an aspect, a pharmaceutical composition provided or administered herein comprises fecal material or a fecal floral preparation in a lyophilized, crude, semi-purified or purified formulation.

In an aspect, a preparation of uncultured fecal bacteria included in a pharmaceutical composition comprises highly refined or purified fecal flora, e.g., substantially free of non-floral fecal material. In an aspect, a fecal microbiota (comprising uncultured fecal bacteria) harvested from a donor can be further processed, e.g., to undergo microfiltration before, after, or before and after sieving. In another aspect, a highly purified fecal microbiota product is ultra-filtrated to remove large molecules but retain the therapeutic microflora, e.g., bacteria.

In another aspect, a preparation of uncultured fecal bacteria incorporated into a pharmaceutical composition described herein comprises or consists essentially of a substantially isolated or a purified fecal flora or entire (or substantially entire) microbiota that is (or comprises) an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material; or, a substantially isolated, purified, or substantially entire microbiota as described in Sadowsky et al., WO 2012/122478 A1, or as described in Borody et al., WO 2012/016287 A2.

In an aspect, a preparation of uncultured fecal bacteria included in a pharmaceutical composition comprises the substantially entire fecal microbiota of stool of a donor. In another aspect, uncultured fecal bacteria of a pharmaceutical composition comprise no antibiotic resistant bacteria. In another aspect, a pharmaceutical composition comprises a preparation of uncultured fecal bacteria largely free of extraneous matter (e.g., non-living matter including acellular matter such as residual fiber, DNA, RNA, viral coat material, non-viable material; and living matter such as eukaryotic cells from the donor of the fecal matter).

In an aspect, uncultured fecal bacteria included in a pharmaceutical composition are derived from a disease-screened stool sample of a human donor. In an aspect, a stool sample does not include an antibiotic resistant population. For example, a composition can comprise a preparation of viable flora which in proportional content can resemble normal healthy human fecal flora which does not include antibiotic resistant populations.

In one aspect, a preparation of uncultured fecal bacteria described and used herein comprises one or more, two or more, three or more, four or more, or five or more live fecal microorganisms selected from the group consisting of Acidaminococcus, Akkermansia, Alistipes, Anaerotruncus, Bacteroides, Bifidobacterium, Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella. In one aspect, a preparation of uncultured fecal bacteria comprises one or more, two or more, three or more, four or more, or five or more live fecal microorganisms selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, Staphylococcus epidermidis, Eubacterium limosum, Tissirella praeacuta, Fusobacterium mortiferum I, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridliformis, Bacteroides coagulans, Prevotella oxalis, Prevotella ruminicola, Odoribacter splanchnicus, and Desuifomonas pigra.

In one aspect, a preparation of uncultured fecal bacteria described and used herein comprises Faecalibacterim prausnitzii. In another aspect, a preparation of uncultured fecal bacteria described and used herein comprises Eubacterium rectale. In another aspect, a preparation of uncultured fecal bacteria described and used herein comprises Faecalibacterim prausnitzii and Eubacterium rectale. In yet another aspect, a preparation of uncultured fecal bacteria comprises one or more major butyrate-producing bacteria selected from the group consisting of Eubacterium rectale, Roseburia intestinalis, Roseburia faecis, Roseburia hominis, Roseburia inulinivorans, Butyrivibrio fibrisolvens, Eubacterium ramulus, Eubacterim hallii, Anaerostipes caccae, Coprococcus catus GD/7, Coprococcus eutactus L2-50, Coprococcus comes A2-232, Eubacterium cylindroides, Faecalibacterium prausnitzii, Subdoligranulum variabile, and Anaerotruncus colihominis. In another aspect, a preparation of uncultured fecal bacteria comprises a bacteria species of Clostridium XIVa and Clostridium IV clusters.

In one aspect, a preparation of uncultured fecal bacteria described herein comprises one or more SCFA-producing bacterial strains. In aspects, a preparation of fecal bacteria includes at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 45, at least 50, or greater than 50 SCFA-producing bacterial strains.

In an aspect, a pharmaceutical composition can comprise a preparation of uncultured fecal bacteria selected on the basis of the presence of one or more SCFA-producing bacterial strains in the preparation (or in stool from which the preparation is produced), or on the basis of a relative abundance of one or more SCFA-producing bacterial strains in the preparation (or in stool from which the preparation is produced) at or above a threshold relative abundance. In various aspects, a pharmaceutical composition comprises a preparation of uncultured fecal bacteria comprising one or more SCFA-producing bacterial strains at a relative abundance in the preparation of at least 0.5, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, or greater than 80%.

In one aspect, a preparation of uncultured fecal bacteria described and used herein lacks or is substantially devoid of one or more, two or more, three or more, four or more, or five or more live fecal microorganisms selected from the group consisting of Acidaminococcus, Akkermansia, Alistipes, Anaerotruncus, Bacteroides, Bifidobacterium, Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella. In one aspect, a preparation of uncultured fecal bacteria lacks or is substantially devoid of one or more, two or more, three or more, four or more, or five or live more fecal microorganisms selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, Staphylococcus epidermidis, Eubacterium limosum, Tissirella praeacuta, Fusobacterium mortiferum I, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridliformis, Bacteroides coagulans, Prevotella oxalis, Prevotella ruminicola, Odoribacter splanchnicus, and Desuifomonas pigra.

In an aspect, uncultured fecal bacteria for incorporation into a pharmaceutical composition comprise non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum. In an aspect, a pharmaceutical composition comprises one or more, two or more, three or more, or four or more non-pathogenic Bacteroides species selected from the group of Bacteroides coprocola, Bacteroides plebeius, Bacteroides massiliensis, Bacteroides vulgatus, Bacteroides helcogenes, Bacteroides pyogenes, Bacteroides tectus, Bacteroides uniformis, Bacteroides stercoris, Bacteroides eggerthii, Bacteroides finegoldii, Bacteroides thetaiotaomicron, Bacteroides ovatus, Bacteroides acidifaciens, Bacteroides caccae, Bacteroides nordii, Bacteroides salyersiae, Bacteroides fragilis, Bacteroides intestinalis, Bacteroides coprosuis, Bacteroides distasonis, Bacteroides goldsteinii, Bacteroides merdae, Bacteroides forsythus, Bacteroides splanchnicus, Bacteroides capillosus, Bacteroides cellulosolvens, and Bacteroides ureolyticus.

In an aspect, a preparation of uncultured fecal bacteria extracted from stool of a donor comprises all (100%) of the bacterial strains originally present in the stool of the donor. In an aspect, a preparation of uncultured fecal bacteria extracted from stool of a donor comprises 99.9% of the bacterial strains originally present in the stool of the donor. In an aspect, a preparation of uncultured fecal bacteria extracted from stool of a donor comprises 99.8, 99.7, 99.6, 99.5, 99.4, 99.3, 99.2, 99.1, 99, 98, 97, 96, 95, 94, 93, 92, 91, 90, 89, 88, 87, 85, 84, 83, 82, 81, 80, 75, 70, 65, 60, 55, 50, 45, or 40% of the bacterial strains originally present in the stool of the donor.

In an aspect, a pharmaceutical composition disclosed herein comprises a sterile fecal filtrate or a non-cellular fecal filtrate. In one aspect, a sterile fecal filtrate originates from a donor stool. In another aspect, a sterile fecal filtrate originates from cultured microorganisms. In another aspect, a sterile fecal filtrate comprises a non-cellular non-particulate fecal component. In one aspect, a sterile fecal filtrate is made as described in WO2014/078911, published May 30, 2014. In another aspect, a sterile fecal filtrate is made as described in Ott et al., Gastroenterology 152:799-911(2017).

In one aspect, a fecal filtrate comprises secreted, excreted or otherwise liquid components or a microbiota, e.g., biologically active molecules (BAMs), which can be antibiotics or anti-inflammatories, are preserved, retained or reconstituted in a flora extract.

In one aspect, preparation of a fecal filtrate comprises receiving stool from a donor, homogenizing and centrifuging the stool, and then filtering with very high-level filtration using e.g., either metal sieving or Millipore filters, or equivalent, to ultimately permit only cells of bacterial origin to remain, e.g., often less than about 5 micrometers diameter. After the initial centrifugation, the solid material can be separated from the liquid, and the solid ban then be filtered in progressively reducing size filters and tangential filters, e.g., using a Millipore filtration, and optionally, also comprising use of nano-membrane filtering. The filtering can also be done by sieves as described in WO 2012/122478, but in contrast using sieves that are smaller than 0.0120 mm, down to about 0.0110 mm, which ultimately result in having only bacterial cells present.

The supernatant separated during centrifugation can in some aspects be filtered progressively in a filtering, e.g., a Millipore filtering or equivalent systems, to produce a liquid which is finely filtered through an about 0.22 micron filter. This removes all particulate matter including all living matter, including bacteria and viruses. The product then is sterile, but the aim is to remove the bacteria but to keep their secretions, especially antimicrobial bacteriocins, bacteria-derived cytokine-like products and all accompanying Biologically Active Molecules (BAMs), including: thuricin (which is secreted by bacilli in donor stools), bacteriocins (including colicin, troudulixine or putaindicine, or microcin or subtilosin A), lanbiotics (including nisin, subtilin, epidermin, mutacin, mersacidin, actagardine, cinnamycin), lacticins and other antimicrobial or anti-inflammatory compounds.

In one aspect, a pharmaceutical composition comprises reconstituted fecal flora consisting essentially of a combination of a purified fecal microbiota (e.g., a preparation of uncultured fecal bacteria) and a non-cellular fecal filtrate. In another aspect, a pharmaceutical composition comprises a purified fecal microbiota (e.g., a preparation of uncultured fecal bacteria) supplemented with one or more non-cellular non-particulate fecal components. In one aspect, a pharmaceutical composition comprises one or more non-cellular non-particulate fecal components. In one aspect, one or more non-cellular non-particulate fecal components comprise synthetic molecules, biologically active molecules produced by a fecal microorganism, or both. In another aspect, one or more non-cellular non-particulate fecal components comprise biologically active proteins or peptides, micronutrients, fats, sugars, small carbohydrates, trace elements, mineral salts, ash, mucous, amino acids, nutrients, vitamins, minerals, or any combination thereof. In one aspect, one or more non-cellular non-particulate fecal components comprise one or more biologically active molecules selected from the group consisting of bacteriocin, lanbiotic, and lacticin. In another aspect, one or more non-cellular non-particulate fecal components comprise one or more bacteriocins selected from the group consisting of colicin, troudulixine, putaindicine, microcin, and subtilosin A. In one aspect, one or more non-cellular non-particulate fecal components comprise one or more lanbiotics selected from the group consisting of thuricin, nisin, subtilin, epidermin, mutacin, mersacidin, actagardine, and cinnamycin. In another aspect, one or more non-cellular non-particulate fecal components comprise an anti-spore compound, an antimicrobial compound, an anti-inflammatory compound, or any combination thereof. In a further aspect, one or more non-cellular non-particulate fecal components comprise an interleukin, a cytokine, a leukotriene, an eicosanoid, or any combination thereof.

In another aspect, a pharmaceutical composition comprises both uncultured fecal bacteria, e.g., a partial or a complete representation of the human GI microbiota, and an isolated, processed, filtered, concentrated, reconstituted and/or artificial liquid component (e.g., fecal filtrate) of the flora (the microbiota) which comprises, among others ingredients, bacterial secretory products such as e.g., bacteriocins (proteinaceous toxins produced by bacteria, including colicin, troudulixine or putaindicine, or microcin or subtilosin A), lanbiotics (a class of peptide antibiotics that contain a characteristic polycyclic thioether amino acid lanthionine or methyllanthionine, and unsaturated amino acids dehydroalanine and 2-aminoisobutyric acid; which include thuricin (which is secreted by bacilli in donor stools), nisin, subtilin, epidermin, mutacin, mersacidin, actagardine, cinnamycin), a lacticin (a family of pore-forming peptidic toxins) and other antimicrobial or anti-inflammatory compounds and/or additional biologically active molecules (BAMs) produced by bacteria or other microorganisms of the microbiota, and/or which are found in the “liquid component” of a microbiota.

In one aspect, a pharmaceutical composition comprising a preparation of uncultured fecal bacteria (e.g., selected to produce one or more SCFAs) is used concurrently with a fecal non-cellular filtrate-based pharmaceutical composition. In another aspect, a patient is treated with a first fecal non-cellular filtrate-based pharmaceutical composition before being given a second pharmaceutical composition comprising uncultured fecal bacteria (e.g., selected to produce one or more SCFAs), or vice versa. In a further aspect, a treatment method comprises three steps: first, antibiotic pretreatment to non-selectively remove infectious pathogen(s); second, a fecal non-cellular filtrate-based treatment step to further suppress selected infectious pathogen(s); and third, treatment with a pharmaceutical composition comprising uncultured fecal bacteria selected to produce one or more SCFAs to re-establish a functional intestinal microbiome.

In an aspect, a composition comprising a preparation of uncultured fecal bacteria that is administered to a subject having or at risk for a disorder (e.g., a disorder that can be treated by delivery of one or more SCFAs to the intestine of the subject) effects a cure, reduction of the symptoms, or a percentage reduction of symptoms of the disorder based on replacement of bacterial cells endogenous to the intestinal flora of the subject with bacterial cells from the administered bacterial preparation. The change of flora can be as “near-complete” as possible. Typically, the change in enteric flora comprises introduction of an array of flora derived from the stool of a healthy human donor into the gastro-intestinal system of the subject, which can substantially or completely displace pathogenic enteric flora in a patient requiring such treatment (e.g., an IBD or colorectal cancer patient). In an aspect, engraftment of the administered uncultured fecal bacteria in the intestine of the subject results in production and secretion of one or more SCFAs by the engrafted bacteria, which as described herein can produce therapeutic effects in the subject.

The pharmaceutical compositions described herein can comprise microbes, e.g. bacteria, derived from a stool sample of a donor, e.g. a healthy human donor. In an aspect, a composition incorporates uncultured fecal bacteria derived from all or a portion of a fecal microbiota of a stool sample of a healthy human donor. For example, a composition can incorporate a substantially complete fecal microbiota of a stool sample of a healthy human donor. In an aspect, a composition incorporates a bacterial isolate of a fecal microbiota, wherein the bacterial isolate has been purified and/or cultured from all or a portion of the fecal microbiota of a stool sample from a healthy human donor. The harvesting, extraction and/or purification of a fecal microbiota from a stool sample can thus be performed to prepare a composition comprising at least one of uncultured fecal bacteria or a bacterial isolate.

In one aspect, an exemplary fecal microbiota for use in preparing a composition described herein (e.g., comprising uncultured fecal bacteria) comprises starting material from a human donor. In another aspect, an exemplary fecal microbiota comprises material from one or more healthy human donors. In yet another aspect, an exemplary fecal microbiota comprises starting material from a pool of known, defined donors. In another aspect, a donor is an adult male. In a further aspect, a donor is an adult female. In yet another aspect, a donor is an adolescent male. In another aspect, a donor is an adolescent female. In another aspect, a donor is a female toddler. In another aspect, a donor is a male toddler. In another aspect, a donor is healthy. In one aspect, a human donor is a child below about 18, 15, 12, 10, 8, 6, 4, 3, 2, or 1-year-old. In another aspect, a human donor is an elderly individual. In a further aspect, a human donor is an individual above about 30, 35, 40, 45, 50, 55, 60, 65, 70, 75, 80, 85, 90, or 95 years old. In another aspect, a donor is between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a donor is a young old individual (65-74 years). In one aspect, a donor is a middle old individual (75-84 years). In one aspect, a donor is an old individual (>85 years). In yet another aspect, a donor is a carefully screened, healthy, neurotypical human.

In an aspect, a fecal donor can be prescreened for its fecal microbiome profile. In another aspect, a fecal donor can be selected on the basis of the presence of one or more bacterial taxa (e.g., phylum, class, order, family, genus, species or strain) in the donor's stool. In another aspect, a fecal donor can be selected on the basis of the presence of one or more bacterial taxa (e.g., phylum, class, order, family, genus, species or strain) in the donor's stool at a level above a threshold relative abundance. In an aspect, a fecal donor can be selected on the basis of the presence or relative abundance of one or more SCFA-producing bacterial strains in the stool of the donor. In another aspect, a fecal donor can be selected on the basis of the presence or relative abundance of one or more bacterial taxa (e.g., phylum, class, order, family, genus, species or strain) of bile acid transforming strains.

In an aspect, a relative abundance of one or more bacterial strains in stool of a donor is increased by ingestion by the donor of a prebiotic and/or probiotic that promotes the proliferation or presence of the bacterial strain or strains, compared to a relative abundance of the bacterial strain or strains in the absence of ingesting the probiotic and/or prebiotic. In an aspect, a fecal donor can ingest a stimulant for one or more SCFA-producing bacterial strains (e.g., as bacterial isolates) prior to donation of a stool for use in preparing uncultured fecal bacteria for incorporation into a pharmaceutical composition. Examples of stimulants for SCFA-producing bacterial strains include probiotics and prebiotics. In an aspect, a fecal donor can ingest a probiotic comprising one or more SCFA-producing bacterial strains (e.g., as bacterial isolates) prior to donation of a stool for use in preparing uncultured fecal bacteria for incorporation into a pharmaceutical composition. In another aspect, a fecal donor can ingest a prebiotic prior to donation of a stool for use in preparing uncultured fecal bacteria for incorporation into a pharmaceutical composition. In an aspect, administration of a prebiotic to a donor increases a relative abundance of one or more SCFA-producing bacterial strains in the stool of the donor. In an aspect, a prebiotic administered to a donor prior to collection of stool from the donor comprises, for example, an amino acid (e.g., valine, leucine, isoleucine), lactic acid, ammonium nitrate, amylose, barley mulch, biotin, carbonate, cellulose, chitin, choline, fructooligosaccharides (FOSs), fructose, glucose, glycerol, heteropolysaccharide, histidine, homopolysaccharide, hydroxyapatite, inulin, isomaltulose, lactose, lactulose, maltodextrins, maltose, nitrogen, oligodextrose, oligofructose, oligofructose-enriched inulin, an oligosaccharide (e.g. comprising a galactooligosaccharide (GOS), trans-galactooligosaccharide, fructooligosaccharide (FOS), xylooligosaccharides (XOS), mannooligosaccharide, or chitooligosaccharide), pectin, phosphate salts, phosphorus, polydextroses, polyols, potash, potassium, sodium nitrate, starch, sucrose, sulfur, sun fiber, tagatose, thiamine, trehalose, vitamins, a water-soluble carbohydrate, a fermentable polysaccharide, a dietary fiber, resistant starch, barley, white navy bean powder, or a combination thereof.

In an aspect, a carefully screened donor undergoes a complete medical history and physical exam. Donors are excluded if they have a risk of infectious agents. Additional exclusion criteria comprise the following:
1. Known viral infection with Hepatitis B, C or HIV
2. Known exposure to HIV or viral hepatitis at any time
3. High risk behaviors including sex for drugs or money, men who have sex with men, more than one sexual partner in the preceding 12 months, any past use of intravenous drugs or intranasal cocaine, history of incarceration.
4. Tattoo or body piercing within 12 months.
5. Travel to areas of the world where risk of traveler's diarrhea is higher than the US.
6. Current communicable disease, e.g., upper respiratory viral infection.
7. History of irritable bowel syndrome. Specific symptoms can include frequent abdominal cramps, excessive gas, bloating, abdominal distension, fecal urgency, diarrhea, constipation.
8. History of inflammatory bowel disease such as Crohn's disease, ulcerative colitis, microscopic colitis.
9. Chronic diarrhea.
10. Chronic constipation or use of laxatives.
11. History of gastrointestinal malignancy or known colon polyposis.
12. History of any abdominal surgery, e.g., gastric bypass, intestinal resection, appendectomy, cholecystectomy, etc.
13. Use of Probiotics or any other over the counter aids used by the potential donor for purpose of regulating digestion. Yogurt and kefir products are allowed if taken merely as food rather than nutritional supplements.
14. Antibiotics for any indication within the preceding 6 months.
15. Any prescribed immunosuppressive or anti-neoplastic medications.
16. Metabolic Syndrome, established or emerging. Criteria used for definition here are stricter than any established criteria. These include history of increased blood pressure, history of diabetes or glucose intolerance.
17. Known systemic autoimmunity, e.g., connective tissue disease, multiple sclerosis.
18. Known atopic diseases including asthma or eczema.
19. Chronic pain syndromes including fibromyalgia, chronic fatigue syndrome.
20. Ongoing (even if intermittent) use of any prescribed medications, including inhalers or topical creams and ointments.
21. Neurologic, neurodevelopmental, and neurodegenerative disorders including autism, Parkinson's disease.
22. General. Body mass index>26 kg/m2, central obesity defined by waste:hip ratio >0.85 (male) and >0.80 (female).
23. Blood pressure >135 mmHg systolic and >85 mmHg diastolic.
24. Skin—presence of a rash, tattoos or body piercing placed within a year, or jaundice
25. Enlarged lymph nodes.
26. Wheezing on auscultation.
27. Hepatomegaly or stigmata of liver disease.
28. Swollen or tender joints. Muscle weakness.
29. Abnormal neurologic examination.
30. Positive stool Clostridium difficile toxin B tested by PCR.
31. Positive stool cultures for any of the routine pathogens including Salmonella, Shigella, Yersinia, Campylobacter, E. coli 0157:H7.
32. Abnormal ova and parasites examination.
33. Positive Giardia, Cryptosporidium, or Helicobacter pylori antigens.
34. Positive screening for any viral illnesses, including HIV 1 and 2, Viral Hepatitis A IgM, Hepatitis surface antigen and core Ab.
35. Abnormal RPR (screen for syphilis).
36. Any abnormal liver function tests including alkaline phosphatase, aspartate aminotransaminase, alanine aminotransferase.
37. Raised serum triglycerides >150 mg/D1
38. HDL cholesterol <40 mg/dL (males) and <50 mg/dL (females)
39. High sensitivity CRP >2.4 mg/L
40. Raised fasting plasma glucose (>100 mg/dL)

In one aspect, provided herein is a process for collecting and processing a stool sample to give rise to a preparation of uncultured fecal bacteria. The process can comprise first collecting a stool sample from one or more healthy (e.g., screened) donor(s). In one aspect, a fresh stool is transported via a stool collection device, which can provide or comprises a suitably oxygen free (or substantially oxygen free) appropriate container. In one aspect, the container can be made oxygen free by e.g., incorporating into the container a built in or clipped-on oxygen-scavenging mechanism, e.g., oxygen scavenging pellets as described e.g., in U.S. Pat. No. 7,541,091. In another aspect, the container itself is made of an oxygen scavenging material, e.g., oxygen scavenging iron, e.g., as described by O2BLOCK™, or equivalents, which uses a purified and modified layered clay as a performance-enhancing carrier of oxygen-scavenging iron; the active iron is dispersed directly in the polymer. In one aspect, oxygen-scavenging polymers are used to make the container itself or to coat the container, or as pellets to be added; e.g., as described in U.S. Pat. App. Pub. 20110045222, describing polymer blends having one or more unsaturated olefinic homopolymers or copolymers; one or more polyamide homopolymers or copolymers; one or more polyethylene terephthalate homopolymers or copolymers; that exhibit oxygen-scavenging activity. In one aspect, oxygen-scavenging polymers are used to make the container itself or to coat the container, or as pellets to be added; e.g., as described in U.S. Pat. App. Pub. 20110008554, describing compositions comprising a polyester, a copolyester ether and an oxidation catalyst, wherein the copolyester ether comprises a polyether segment comprising poly(tetramethylene-co-alkylene ether). In one aspect, oxygen-scavenging polymers are used to make the container itself or to coat the container, or as pellets to be added; e.g., as described in U.S. Pat. App. Pub. 201000255231, describing a dispersed iron/salt particle in a polymer matrix, and an oxygen scavenging film with oxygen scavenging particulates.

Alternatively, in addition to or in place of the oxygen-scavenging mechanism, the air in the container can be replaced (completely or substantially) with nitrogen and/or other inert non-reactive gas or gases. In one aspect, the container simulates (creates) partially, substantially or completely an anaerobic environment.

In one aspect, the stool (e.g., fecal sample) is held in an aesthetically acceptable container that will not leak nor smell yet maintain an anaerobic environment. In one aspect, the container is sterile before receiving the fecal flora.

In one aspect, a stool sample provided herein is maintained at room temperature during most or all of its transportation and/or storage at e.g., a “stool bank”. For example, once delivered to a “processing stool bank” it is stored at ambient temperature, e.g., room temperature. In one aspect, stabilizing agents, such as glycerol, are added to the harvested and/or stored material.

In one aspect, the stool is tested for various pathogens, as noted above. In one aspect, once cleared of infective agents, a stool sample is homogenized and filtered to remove large particles of matter. In one aspect, the stool is subdivided into desired volumes, e.g., which can be between 5 cc and 3 or more liters. For example, in one aspect, a container comprises a 50 gram (g) stool, which can be held in an appropriate oxygen resistant plastic, e.g., a metallized polyethylene terephthalate polyester film, or a metallized MYLAR™.

In one aspect, the stool is subject to homogenization by for example, mixing, agitating, stirring or shaking. In certain aspects, a stool sample is diluted with a homogenization buffer prior to homogenization. A homogenization buffer can, for example, contain a cryoprotectant (e.g., trehalose), an antioxidant or reducing agent (e.g., cysteine), and a buffer (e.g., 0.25×PBS at pH 7.4).

In one aspect, to separate the non-bacterial components from the fecal microbiota, the stool can be homogenized and filtered from rough particulate matter. In one aspect, the microscopic fiber/nonliving matter is then separated from the bacteria. Several methods can be used, including e.g., recurrent filtration with filter sizes, e.g., progressively coming down to the size of a typical bacterium.

In one aspect, different filters are used to isolate bacterial sp., or a technique as used by Williams in WO 2011/033310A1, which uses a crude technique of filtration with a gauze.

In one aspect, a filtration procedure for filtering whole stool is suitably used to reach the highest concentration of almost 100% bacteria. In one aspect, the filtering procedure is a two-step procedure suitably using glass fibre depth filters for initial clarification. In one aspect, the stool is filtered under positive pressure. In one aspect, this would be using a combination or sandwich configuration with a 30 micron PVDF filter. In one aspect, this sandwich procedure will be filtering the product under positive pressure. Later, membrane concentration can, in one aspect, be used as another step to reduce the volume of the filtrate. In one aspect, this can be done prior to freeze drying or spray drying under nitrogen cover.

Alternative membranes that can be used for filtration include, but not limited to, nylon filters, cellulose nitrate filters, polyethersulfone (PES) filters, polytetrafluorethylene (PTFE) filters, TEFLON™ filters, mixed cellulose Ester filters, polycarbonate filters, polypropylene filters, Polyvinylchloride (PVC) filters or quartz filters. Various combinations of these can be used to achieve a high purity of bacteria with solids and liquid removed.

In another aspect, a pharmaceutical composition comprises a bacterial mixture comprising a preparation of uncultured fecal bacteria supplemented, spiked, enriched, or enhanced with one or more bacterial isolates (e.g., a probiotic). For example, a bacterial mixture can comprise a preparation of uncultured fecal bacteria spiked with one or more bacterial isolates comprising one or more SCFA-producing bacterial strains. By enriching or spiking a preparation of uncultured fecal bacteria derived from a stool sample (e.g., a fecal microbiota) of a healthy donor with one or more non-pathogenic bacterial isolates (e.g. comprising one or more SCFA-producing bacterial strains), a bacterial mixture can be produced in which the amount of a particular bacterial strain or strains (i.e. the spiked-in bacterial isolate(s)) can be accounted for and precisely controlled. Without wishing to be bound by theory, this is advantageous, for example, where a bacterial isolate mixed with the preparation of uncultured fecal bacteria is important for or involved in the treatment of a disorder of a subject (e.g., a disorder that can be treated by delivery of SCFA to the subject's intestine), but insufficient on its own to generate a complete or optimal treatment response in the subject. Unlike probiotics, administration to a subject of one or more bacterial isolates together with a preparation of uncultured fecal bacteria (i.e., derived from a healthy donor) provides the subject with the advantage of the administered bacterial isolate combined with multi-factorial benefits conferred by the additional fecal bacterial strains present in the uncultured preparation. These additional fecal bacterial strains may combine to, for example, provide for the necessary context or interactions (e.g. via one or more released factors) to enable the bacterial isolate to induce an optimal response in the subject, or may directly induce a response in the subject that combines and/or synergizes with a response induced by the bacterial isolate to treat the subject. Accordingly, in certain aspects, a pharmaceutical composition comprising a mixture of one or more bacterial isolates and a preparation of uncultured fecal bacteria can be more effective in treating a disorder of a subject (e.g., a disorder that can be treated by delivery of SCFA to the subject's intestine) than a composition comprising either the bacterial isolate or the preparation of uncultured fecal bacteria alone.

Pharmaceutical Compositions, Formulations, and Administration

Described herein are pharmaceutical compositions comprising a bacterial mixture comprising a preparation of uncultured fecal bacteria in various formulations. Any pharmaceutical composition (and/or additional therapeutic agents) described herein can take the form of tablets, pills, pellets, capsules, capsules containing liquids, capsules containing multiparticulates, powders, solutions, emulsion, drops, suppositories, emulsions, aerosols, sprays, suspensions, delayed-release formulations, sustained-release formulations, controlled-release formulations, or any other form suitable for use.

The formulations comprising the pharmaceutical compositions described herein can conveniently be presented in unit dosage forms. For example, the dosage forms can be prepared by methods which include the step of bringing the therapeutic agents into association with a carrier, which constitutes one or more accessory ingredients. For example, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by press tableting).

In another aspect, a pharmaceutical composition can include a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and a combination thereof. In another aspect, a pharmaceutical composition can contain auxiliary substances such as wetting or emulsifying agents, stabilizing or pH buffering agents. In an aspect, a pharmaceutical composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a pharmaceutical composition contains about 2%-70%, 5%-60%, 10%-50%, 15%-40%, 20%-30%, 25%-60%, 30%-60%, or 35%-60% of active ingredient.

In an aspect, a pharmaceutical composition can include or be incorporated into tablets, drenches, boluses, capsules or premixes. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, for preparing solid compositions such as tablets, an active ingredient is mixed with a pharmaceutical carrier, e.g., conventional tableting ingredients such as cornstarch, lactose, sucrose, sorbitol, talc, stearic acid, magnesium stearate, dicalcium phosphate or gums, or other pharmaceutical diluents, e.g. water, to form a solid preformulation composition containing a homogeneous mixture of a composition described herein. When referring to these preformulation compositions as homogeneous, it is meant that the active ingredient is dispersed evenly throughout the composition so that the composition can be readily subdivided into equally effective unit dosage forms such as tablets, pills and capsules. This solid preformulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ CFUs). A pharmaceutical composition described herein can be flavored.

In an aspect, a pharmaceutical composition comprising a bacterial mixture described herein (and optionally one or more additional therapeutic agents) is formulated as a composition adapted for a mode of administration described herein.

In various aspects, the administration of the pharmaceutical compositions is any one of oral, intravenous, intraperitoneal, and parenteral. For example, routes of administration include, but are not limited to, oral, intraperitoneal, intravenous, intramuscular, or rectal. In various aspects, the administration of the pharmaceutical compositions is oral, naso-gastric, antegrade gastrointestinal, retrograde gastrointestinal, endoscopic, or enemic.

In an aspect, a pharmaceutical composition described herein can be formulated as a composition adapted for oral administration. Compositions for oral delivery can be in the form of tablets, lozenges, aqueous or oily suspensions, granules, powders, sprinkles, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration to provide sustained delivery of the bacterial mixture over an extended period of time. Selectively permeable membranes surrounding an osmotically active agent are also suitable for orally administered compositions. In these latter platforms, fluid from the environment surrounding the capsule is imbibed by a driving compound, which swells to displace the agent or agent composition through an aperture. These delivery platforms can provide an essentially zero order delivery profile as opposed to the spiked profiles of immediate release formulations. A time-delay material, such as glycerol monostearate or glycerol stearate, can also be useful. Oral compositions can include standard excipients such as mannitol, lactose, starch, magnesium stearate, sodium saccharin, cellulose, ethacrylic acid and derivative polymers thereof, and magnesium carbonate. In an aspect, the excipients are of pharmaceutical grade. Suspensions, in addition to the active compounds, can contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

In various aspects, a pharmaceutical composition is formulated as a solid dosage form such as a tablets, dispersible powder, granule, or capsule. In an aspect, the pharmaceutical composition is formulated as a capsule. In another aspect, the pharmaceutical composition is formulated as a tablet. In yet another aspect, the pharmaceutical composition is formulated as a soft-gel capsule. In a further aspect, the pharmaceutical composition is formulated as a gelatin capsule.

In an aspect, a pharmaceutical composition is in the form of: an enema composition which can be reconstituted with an appropriate diluent; an enteric-coated capsule; an enteric-coated microcapsule; an acid-resistant tablet; an acid-resistant capsules; an acid-resistant microcapsule; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated and/or acid-resistant microcapsules of the composition, powder, jelly, or liquid.

In various aspects, formulations can additionally comprise a pharmaceutically acceptable carrier or excipient. As one skilled in the art will recognize, the formulations can be in any suitable form appropriate for the desired use and route of administration.

In some dosage forms, a pharmaceutical composition described herein is mixed with at least one inert, pharmaceutically acceptable excipient or carrier such as sodium citrate, dicalcium phosphate, etc., and/or a) fillers or extenders such as starches, lactose, sucrose, glucose, mannitol, silicic acid, microcrystalline cellulose, and Bakers Special Sugar, etc., b) binders such as, for example, carboxymethylcellulose, alginates, gelatin, polyvinylpyrrolidone, sucrose, acacia, polyvinyl alcohol, polyvinylpyrrolidone, methylcellulose, hydroxypropyl cellulose (HPC), and hydroxymethyl cellulose etc., c) humectants such as glycerol, etc., d) disintegrating agents such as agar-agar, calcium carbonate, potato or tapioca starch, alginic acid, certain silicates, sodium carbonate, cross-linked polymers such as crospovidone (cross-linked polyvinylpyrrolidone), croscarmellose sodium (cross-linked sodium carboxymethylcellulose), sodium starch glycolate, etc., e) solution retarding agents such as paraffin, etc., f) absorption accelerators such as quaternary ammonium compounds, etc., g) wetting agents such as, for example, cetyl alcohol and glycerol monostearate, etc., h) absorbents such as kaolin and bentonite clay, etc., and i) lubricants such as talc, calcium stearate, magnesium stearate, solid polyethylene glycols, sodium lauryl sulfate, glyceryl behenate, etc., and mixtures of such excipients. One of skill in the art will recognize that particular excipients can have two or more functions in the oral dosage form. In the case of an oral dosage form, for example, a capsule or a tablet, the dosage form can also comprise buffering agents.

In an aspect, a pharmaceutical composition comprising a bacterial mixture is combined with one or more pharmaceutically acceptable cryoprotectants, lyoprotectants, binders, disintegrants, excipients, fillers, and/or preservatives, acid suppressants, antacids, H2 antagonists, and proton pump inhibitors, or combinations thereof.

In an aspect, a pharmaceutical composition comprising a bacterial mixture is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a pharmaceutical composition.

In one aspect, a pharmaceutical composition administered herein further comprises an acid suppressant, an antacid, an H2 antagonist, a proton pump inhibitor or a combination thereof. In one aspect, a pharmaceutical composition administered herein is substantially free of non-living matter. In another aspect, a pharmaceutical composition administered herein substantially free of acellular material selected from the group consisting of residual fiber, DNA, viral coat material, and non-viable material. In another aspect, a pharmaceutical composition administered does not comprise an acid suppressant, an antacid, an H2 antagonist, a proton pump inhibitor or a combination thereof. In yet another aspect, a pharmaceutical composition administered does not comprise an acid suppressant. In another aspect, a pharmaceutical composition administered does not comprise an antacid. In another aspect, a pharmaceutical composition administered does not comprise an H2 antagonist. In another aspect, a pharmaceutical composition administered does not comprise a proton pump inhibitor. In another aspect, a pharmaceutical composition administered does not comprise metoclopramide.

In an aspect, a bacterial mixture is dry, e.g., when it includes lyophilized bacterial cells/spores or comprises dry binders, fillers, and dispersants. Alternately, the bacterial mixture can be aqueous, e.g., when it comprises non-dry binders, fillers, and dispersants.

In an aspect, a bacterial mixture described herein can be subject to lyophilization. As used herein, “lyophilization” or “freeze drying” refers to the process of drying a material by first freezing it and then encouraging the ice within it to sublimate in a vacuum environment.

In one aspect, a bacterial mixture comprises a lyophilized formulation further comprising a reducing agent and/or antioxidant. In certain aspects, the reducing agent comprises cysteine selected from the group consisting of D-cysteine and L-cysteine. In another aspect, cysteine is at a concentration of at least about 0.025%. In one aspect, cysteine is at a concentration of about 0.025%. In another aspect, cysteine is at a concentration of 0.025%. In another aspect, another reducing agent other than cysteine is used in lieu of, or in combination with cysteine. In an aspect, another reducing agent is selected from the group comprising ascorbic acid, sodium ascorbate, thioglycolic acid, sodium sulfite, sodium bisulfite, sodium metabisulfite, potassium metabisulfite, glutathione, methionine, thioglycerol, and alpha tocopherol.

In one aspect, cysteine is at a concentration of at least about 0.005%, at least about 0.01%, at least about 0.015%, at least about 0.02%, at least about 0.025%, at least about 0.03%, at least about 0.035%, at least about 0.04%, at least about 0.045%, at least about 0.05%, at least about 0.055%, at least about 0.06%, at least about 0.065%, at least about 0.07%, at least about 0.075%, at least about 0.08%, at least about 0.085%, at least about 0.09%, at least about 0.095%, at least about 0.1%, at least about 0.12%, at least about 0.14%, at least about 0.16%, at least about 0.18%, at least about 0.2%, at least about 0.25%, at least about 0.3%, at least about 0.4%, at least about 0.5%, at least about 0.6%, at least about 0.7%, at least about 0.8%, at least about 0.9%, at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, or at least about 26%.

In one aspect, a bacterial mixture comprises a cryoprotectant or mixture of cryoprotectants. As used herein, a “cryoprotectant” refers to a substance that is added to a formulation in order to protect an active ingredient during freezing. For example, a cryoprotectant can comprise, consist essentially of, or consist of polyethylene glycol, skim milk, erythritol, arabitol, sorbitol, glucose, fructose, alanine, glycine, proline, sucrose, lactose, ribose, trehalose, dimethyl sulfoxide (DMSO) or equivalent, a glycerol, a polyethylene glycol (PEG) or equivalent, or an amino acid (e.g., alanine, glycine, proline). In an aspect of the present disclosure, a cryoprotectant can be selected from the group comprising 5% Sucrose; 10% Sucrose; 10% Skim milk; 10% Trehalose with 2.5% sucrose; 5% Trehalose with 2.5% sucrose; 5% Mannitol; 5% Mannitol with 0.1% Polysorbate 80; 10% Mannitol; 10% Mannitol with 0.1% Polysorbate 80; 5% Trehalose; 5% Trehalose with 0.1% Polysorbate 80; 10% Trehalose; and 10% Trehalose with 0.1% Polysorbate 80.

In an aspect, a bacterial mixture comprises a lyoprotectant. As used herein, a “lyoprotectant” refers to a substance that is added to a formulation in order to protect an active ingredient during lyophilization. In one aspect, the same substance or the same substance combination is used as both a cryoprotectant and a lyoprotectant. Exemplary lyoprotectants include sugars such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and a combination thereof. In an aspect, a lyoprotectant is a non-reducing sugar, such as trehalose or sucrose. In an aspect, a cryoprotectant or a lyoprotectant consists essentially of, or consists of, one or more substances mentioned in this paragraph and the paragraph above.

In an aspect, a cryoprotectant or a lyoprotectant comprise an intracellular agent, e.g., DMSO, Glycerol, or PEG, which penetrates inside the cell preventing the formation of ice crystals that could result in membrane rupture. In an aspect, a cryoprotectant or a lyoprotectant comprise an extracellular agent, e.g., sucrose, trehalose, or dextrose, which does not penetrate into the cell membrane but acts to improve the osmotic imbalance that occurs during freezing.

In one aspect, the present disclosure provides a pharmaceutical composition comprising a lyophilized fecal microbe preparation comprising a lyophilization formulation comprising at least about 12.5% trehalose.

In an aspect, a lyophilized formulation comprises trehalose. In an aspect, a lyophilized formulation comprises 2% to 30%, 3% to 25%, 4% to 20%, 5% to 15%, 6% to 10%, 2% to 30%, 2% to 25%, 2% to 20%, 2% to 15%, or 2% to 10% trehalose. In an aspect, a lyophilized formulation comprises at least 2%, 3%, 4%, 5%, 6%, 7%, 8%, 9%, 10%, or 15% trehalose. In an aspect, a lyophilized formulation comprises at most 2%, 3%, 4%, 5%, 6%, 7%, 8%, 9%, 10%, or 15% trehalose. In another aspect, a lyophilized formulation comprises about 5% trehalose. In another aspect, a lyophilized formulation comprises trehalose and sucrose. In another aspect, a lyophilized formulation comprises between about 8% and 12% trehalose with between about 1.5% and 3.5% sucrose and between about 0.5% and 1.5% NaCl.

In one aspect, a lyophilization formulation comprises at least about 5%, at least about 7.5%, at least about 10%, at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20%, at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, or at least about 60% of trehalose.

In an aspect, a pharmaceutical composition provided herein, after at least 12 weeks of storage at ambient temperature or lower, is effective for treating or preventing a disorder in a patient by delivering one or more SCFAs to an intestine of the patient. In an aspect, a pharmaceutical composition remains effective after at least 4, 8, 10, 16, 20, 24, 30, 40, 50, 60, 70, 80 or 100 weeks of storage at ambient temperature or lower.

In an aspect, a pharmaceutical composition described herein can be lyophilized or freeze dried and stored at ambient temperatures (e.g., room temperature), at a freezing temperature, or at between about 2° C. and 8° C. In an aspect, freeze-drying allows the majority of cells to remain viable, and produces a powdered form of the product that can be gently pulverized into a powder. The powder, or lyophilized or freeze-dried composition, then can be encapsulated into a carrier, e.g., a tablet, geltab, pill or capsule, e.g., an enteric-coated capsule, or placed into oil-filled capsules for ingestion. Alternatively, the freeze-dried or lyophilized product, or powder, can be reconstituted at ambient temperatures before delivery to an individual in e.g., a fluid, e.g., a sterile fluid, such as saline, a buffer or a media such as a fluid-glucose-cellobiose agar (RGCA) media.

For freeze-drying, in an aspect, bacteria are held in a liquid that will prevent bursting of cells on thawing. This can include various stabilizers, e.g., glycerol and appropriate buffers, and/or ethylene glycol. In an aspect, the cryoprotecting process uses final concentrations of stabilizer(s) of between about 10% and 80%, 20% and 70%, 30% and 60%, or 40% and 50%, depending on the stabilizer(s) used; in an aspect, this helps stabilize proteins by preventing formation of ice crystals that would otherwise destroy protein structures.

In an aspect, stabilizers that help reduce destruction of living bacteria include skim milk, erythritol, arabitol, sorbitol, glucose, fructose and other polyols. Polymers such as dextran and polyethylene glycol can also be used to stabilize bacterial cells.

In an aspect, manufacturing a pharmaceutical composition can comprise steps of: (1) coating the exterior of a dissociated capsule (i.e., comprising separate capsule body and capsule cap) with the exterior enteric coating, (2) filling the capsule body with a bacterial mixture (e.g., comprising a preparation of uncultured fecal bacteria), and (3) closing the capsule cap over the capsule body, thereby encapsulating the bacterial mixture in the enteric-coated capsule.

Optionally, manufacturing a pharmaceutical composition can comprise steps of: (1) coating the exterior of a dissociated capsule (i.e., comprising separate capsule body and capsule cap) with the exterior enteric coating, (2) coating the interior of the dissociated capsule with an interior coating, (3) filling the capsule body with a bacterial mixture (e.g., comprising a preparation of uncultured fecal bacteria), and (4) closing the capsule cap over the capsule body, thereby encapsulating the bacterial mixture in the dual-coated capsule.

Alternately, manufacturing a pharmaceutical composition can comprise step of: (1) coating the interior of the dissociated capsule (i.e., comprising separate capsule body and capsule cap) with an interior coating, (2) coating the exterior of a dissociated capsule with the exterior enteric coating, (3) filling the capsule body with a bacterial mixture (e.g., comprising a preparation of uncultured fecal bacteria), and (4) closing the capsule cap over the capsule body, thereby encapsulating the bacterial mixture in the dual-coated capsule.

In an aspect, one or more additional therapeutic agents can be included in a pharmaceutical composition, and encapsulated by the capsule.

In an aspect, the bodies and caps of gelatin capsules (e.g., size #00) are separated. An exterior enteric coating suspension is prepared by dispersing one or more enteric coating polymers along with other components in a solution. The exterior enteric coating suspension is applied to the exterior of separated capsule bodies and caps, e.g., using a fluid bed Wurster column coater, Fluid Bed Coater, or an equivalent). The capsules are fluidized in the product bowl and the exterior enteric coating suspension is sprayed to produce the outer coating to a target of between about 2 mg/cm2 and 6 mg/cm2, e.g., 3 mg/cm2. After completion of this step, the capsules are set to dry, e.g., between about 8 hours and 24 hours. After drying, exemplary capsules are weighed to calculate weight gain from the exterior enteric coating. Capsules can be inspected for irregularities.

In an aspect, EUDRAGIT® S100 (poly(methacrylic acid, methylmethacrylate)), starch, triethyl citrate, and PlasACRYL™ T20 are dissolved in a solution of water, ethanol, and n-butanol, mixed, and then charged to a suitable spraying device. The solution is then spray coated on the outer surface of the capsule bodies and capsule caps to a target weight gain. The capsule bodies and capsule caps are allowed to dry for about 8 hours to about 24 hours, or longer, e.g., for a week, a month, or more, before further procession, e.g., filling with a bacterial mixture.

In an aspect, it may be desirable to provide an amount of the bacterial mixture to a capsule's cap in addition to providing the composition in the capsule's body. In this aspect, more of the composition will be included in a capsule and/or less air will be contained in a closed capsule.

In an aspect, the interior surface of a capsule comprises an internal coating.

Any of the above-described compositions and materials (e.g., bacterial mixtures, inner coatings, capsules, and outer coatings) can be combined into a pharmaceutical composition described herein. A skilled artisan would know how to select an inner coating; capsule, and outer coating according to his/her present need, which could be based, for example, on a specific bacterial isolate(s) incorporated into a bacterial mixture of the composition and/or the desired delivery location in a subject (e.g., in the colon or small intestine, including the ileum, jejunum or duodenum) of a component of the bacterial mixture (e.g. comprising a preparation of uncultured fecal bacteria, a bacterial isolate and/or an additional therapeutic agent).

Additional relevant teachings are disclosed in WO 2007122374, which is hereby incorporated herein by reference in its entirety.

In an aspect, during the manufacture of a pharmaceutical composition, a pharmaceutically-acceptable cryoprotectant, lyoprotectant, binder, di sintegrant, filler, preservative, acid suppressant, antacid, H2 antagonist, and proton pump inhibitor, or combination thereof can be mixed into the pharmaceutical composition (e.g., comprising a bacterial mixture) to promote desirable properties.

In an aspect, the pharmaceutical composition comprises a surface active agent. Surface active agents suitable for use include, but are not limited to, any pharmaceutically acceptable, non-toxic surfactant. Classes of surfactants suitable for use include, but are not limited to, polyethoxylated fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-olyoxypropylene block copolymers, sorbitan fatty acid esters, lower alcohol fatty acid esters, ionic surfactants, and mixtures thereof. In some aspects, compositions can comprise one or more surfactants including, but not limited to, sodium lauryl sulfate, polysorbate 20, polysorbate 40, polysorbate 60, polysorbate 80, and triethyl citrate.

In an aspect, the pharmaceutical composition comprises pharmaceutically acceptable plasticizers to obtain the desired mechanical properties such as flexibility and hardness. Such plasticizers include, but are not limited to, triacetin, citric acid esters, triethyl citrate, phthalic acid esters, dibutyl sebacate, cetyl alcohol, polyethylene glycols, polysorbates or other plasticizers.

In another aspect, the pharmaceutical composition comprises one or more application solvents. Some of the more common solvents that can be used to apply, for example, a delayed-release coating composition include isopropyl alcohol, acetone, methylene chloride and the like.

In yet another aspect, the pharmaceutical composition comprises one or more alkaline materials. Alkaline material suitable for use in compositions include, but are not limited to, sodium, potassium, calcium, magnesium and aluminum salts of acids such as phosphoric acid, carbonic acid, citric acid and other aluminum/magnesium compounds. In addition, the alkaline material can be selected from antacid materials such as aluminum hydroxides, calcium hydroxides, magnesium hydroxides and magnesium oxide.

Besides inert diluents, the orally administered compositions can also include adjuvants such as sweetening, flavoring, and perfuming agents.

In various aspects, the pharmaceutical compositions are formulated for systemic or local delivery. In an aspect, administration is systemic. In another aspect, it may be desirable to administer locally to the area in need of treatment.

Various methods can be used to formulate and/or deliver a pharmaceutical composition (e.g., comprising a bacterial mixture and/or additional therapeutic agent) described herein to a location of interest. For example, the pharmaceutical compositions can be formulated for delivery to the GI tract. The GI tract includes organs of the digestive system such as mouth, esophagus, stomach, small intestine, duodenum, jejunum, ileum, large intestine and rectum and includes all subsections thereof (e.g. the small intestine may include the duodenum, jejunum and ileum; the large intestine may include the colon transversum, colon descendens, colon ascendens, colon sigmoidenum and cecum). For example, the compositions can be formulated for delivery of one or more active agents to one or more of the stomach, small intestine, large intestine and rectum, or any subsection thereof (e.g. duodenum, jejunum and ileum, colon transversum, colon descendens, colon ascendens, colon sigmoidenum and cecum). In some aspects, the compositions described herein can be formulated for delivery of one or more active agents to the upper or lower GI tract. In an aspect, a composition can be administered to a subject, by, for example, directly or indirectly contacting the mucosal tissues of the GI tract with the composition.

In various aspects, the administration of the pharmaceutical compositions is into the GI tract via, for example, oral delivery, nasogastral tube, intestinal intubation (e.g. an enteral tube or feeding tube such as, for example, a jejunal tube or gastro-jejunal tube, etc.), direct infusion (e.g., duodenal infusion), endoscopy, colonoscopy, or enema.

In one aspect, a method comprises administering a pharmaceutical composition orally, by enema, or via rectal suppository. In one aspect, a pharmaceutical composition administered herein is formulated as an enteric coated (and/or acid-resistant) capsule or microcapsule, or formulated as part of or administered together with a food, a food additive, a dairy-based product, a soy-based product or a derivative thereof, a jelly, a gelatin-based chewable (e.g., gummy), flavored liquid, ice block, ice cream, or a yogurt. In another aspect, a pharmaceutical composition administered herein is formulated as an acid-resistant enteric coated capsule. A pharmaceutical composition can be provided as a powder for sale in combination with a food or drink. A food or drink can be a dairy-based product or a soy-based product. In another aspect, a food or food supplement contains enteric-coated and/or acid-resistant microcapsules containing a pharmaceutical composition.

In an aspect, a pharmaceutical composition comprises a liquid culture. In another aspect, a pharmaceutical composition is homogenized, lyophilized, pulverized and powdered. It can then be infused, dissolved such as in saline, as an enema. Alternatively, the powder can be encapsulated as enteric-coated and/or acid-resistant delayed release capsules for oral administration. In an aspect, the powder can be double encapsulated with acid-resistant/delayed release capsules for oral administration. These capsules can take the form of enteric-coated and/or acid-resistant delayed release microcapsules. A powder can be provided in a palatable form for reconstitution for drinking or for reconstitution as a food additive. In a further aspect, a food is yogurt. In one aspect, a powder can be reconstituted to be infused via naso-duodenal infusion.

In another aspect, a pharmaceutical composition administered herein is in a liquid, frozen, freeze-dried, spray-dried, foam-dried, lyophilized, or powder form. In a further aspect, a pharmaceutical composition administered herein is formulated as a delayed or gradual enteric release form. In another aspect, a pharmaceutical composition administered herein comprises an excipient, a saline, a buffer, a buffering agent, or a fluid-glucose-cellobiose agar (RGCA) media. In another aspect, a pharmaceutical composition administered herein comprises a cryoprotectant. In one aspect, a cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In various aspects, provided herein are modified-release formulations comprising a bacterial mixture (e.g., comprising a preparation of uncultured fecal bacteria), wherein the formulation releases a substantial amount of the bacterial mixture (and optionally additional therapeutic agents) into one or more regions of the GI tract. For example, the formulation can release at least about 60% of the bacterial isolates after the stomach and into one or more regions of the GI tract.

In various aspects, the modified-release formulation can release at least 60% of the bacterial mixture (and optionally additional therapeutic agents) after the stomach into one or more regions of the intestine. For example, the modified-release formulation can release at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial mixture (and optionally additional therapeutic agents) in the intestines.

In various aspects, the modified-release formulation can release at least 60% of the bacterial mixture (and optionally additional therapeutic agents) in the small intestine. For example, the modified-release formulation can release at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial mixture (and optionally additional therapeutic agents) in the small intestine (e.g., one or more of duodenum, jejunum, ileum, and ileocecal junction).

In various aspects, the modified-release formulation can release at least 60% of the bacterial mixture (and optionally additional therapeutic agents) in the large intestine. For example, the modified-release formulation can release at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial isolates (and/or additional therapeutic agents) in the large intestine (e.g., one or more of cecum, ascending, transverse, descending or sigmoid portions of the colon, and rectum).

In some aspects, the pharmaceutical composition is formulated for release in the stomach. In other aspects, the pharmaceutical composition is formulated so as to not substantially release the bacterial mixture in the stomach.

In certain aspects, the modified-release formulation releases the bacterial mixture (and optionally additional therapeutic agents) at a specific pH. For example, in some aspects, the modified-release formulation is substantially stable in an acidic environment and substantially unstable (e.g., dissolves rapidly or is physically unstable) in a near neutral to alkaline environment. In some aspects, stability is indicative of not substantially releasing while instability is indicative of substantially releasing. For example, in some aspects, the modified-release formulation is substantially stable at a pH of about 7.0 or less, or about 6.5 or less, or about 6.0 or less, or about 5.5 or less, or about 5.0 or less, or about 4.5 or less, or about 4.0 or less, or about 3.5 or less, or about 3.0 or less, or about 2.5 or less, or about 2.0 or less, or about 1.5 or less, or about 1.0 or less. In some aspects, the present formulations are stable in lower pH areas and therefore do not substantially release in, for example, the stomach. In some aspects, modified-release formulation is substantially stable at a pH of about 1 to about 4 or lower and substantially unstable at pH values that are greater. In these aspects, the modified-release formulation does not substantially release in the stomach. In these aspects, the modified-release formulation substantially releases in the small intestine (e.g. one or more of the duodenum, jejunum, and ileum) and/or large intestine (e.g. one or more of the cecum, ascending colon, transverse colon, descending colon, and sigmoid colon). In some aspects, modified-release formulation is substantially stable at a pH of about 4 to about 5 or lower and consequentially is substantially unstable at pH values that are greater and therefore is not substantially released in the stomach and/or small intestine (e.g. one or more of the duodenum, jejunum, and ileum). In these aspects, the modified-release formulation substantially releases in the large intestine (e.g. one or more of the cecum, ascending colon, transverse colon, descending colon, and sigmoid colon). In various aspects, the pH values recited herein can be adjusted as known in the art to account for the state of the subject, e.g. whether in a fasting or postprandial state.

In some aspects, the modified-release formulation is substantially stable in gastric fluid and substantially unstable in intestinal fluid and, accordingly, is substantially released in the small intestine (e.g. one or more of the duodenum, jejunum, and ileum) and/or large intestine (e.g. one or more of the cecum, ascending colon, transverse colon, descending colon, and sigmoid colon).

In some aspects, the modified-release formulation is stable in gastric fluid or stable in acidic environments. These modified-release formulations release about 30% or less by weight of the pharmaceutical composition (e.g., comprising a bacterial mixture) in the modified-release formulation in gastric fluid with a pH of about 4 to about 5 or less, or simulated gastric fluid with a pH of about 4 to about 5 or less, in about 15, or about 30, or about 45, or about 60, or about 90 minutes. Modified-release formulations of can release from about 0% to about 30%, from about 0% to about 25%, from about 0% to about 20%, from about 0% to about 15%, from about 0% to about 10%, about 5% to about 30%, from about 5% to about 25%, from about 5% to about 20%, from about 5% to about 15%, from about 5% to about 10% by weight of the composition in the modified-release formulation in gastric fluid with a pH of 4-5, or less or simulated gastric fluid with a pH of 4-5 or less, in about 15, or about 30, or about 45, or about 60, or about 90 minutes. Modified-release formulations can release about 1%, about 2%, about 3%, about 4%, about 5%, about 6%, about 7%, about 8%, about 9%, or about 10% by weight of the total composition in the modified-release formulation in gastric fluid with a pH of 5 or less, or simulated gastric fluid with a pH of 5 or less, in about 15, or about 30, or about 45, or about 60, or about 90 minutes.

In some aspects, the modified-release formulation is unstable in intestinal fluid. These modified-release formulations release about 70% or more by weight of the bacterial mixture and/or additional therapeutic agent in the modified-release formulation in intestinal fluid or simulated intestinal fluid in about 15, or about 30, or about 45, or about 60, or about 90 minutes. In some aspects, the modified-release formulation is unstable in near neutral to alkaline environments. These modified-release formulations release about 70% or more by weight of the bacterial mixture and/or additional therapeutic agent in the modified-release formulation in intestinal fluid with a pH of about 4-5 or greater, or simulated intestinal fluid with a pH of about 4-5 or greater, in about 15, or about 30, or about 45, or about 60, or about 90 minutes. A modified-release formulation that is unstable in near neutral or alkaline environments can release 70% or more by weight of the pharmaceutical composition (e.g., comprising a microbial cocktail) in the modified-release formulation in a fluid having a pH greater than about 5 (e.g., a fluid having a pH of from about 5 to about 14, from about 6 to about 14, from about 7 to about 14, from about 8 to about 14, from about 9 to about 14, from about 10 to about 14, or from about 11 to about 14) in from about 5 minutes to about 90 minutes, or from about 10 minutes to about 90 minutes, or from about 15 minutes to about 90 minutes, or from about 20 minutes to about 90 minutes, or from about 25 minutes to about 90 minutes, or from about 30 minutes to about 90 minutes, or from about 5 minutes to about 60 minutes, or from about 10 minutes to about 60 minutes, or from about 15 minutes to about 60 minutes, or from about 20 minutes to about 60 minutes, or from about 25 minutes to about 90 minutes, or from about 30 minutes to about 60 minutes.

Examples of simulated gastric fluid and simulated intestinal fluid include, but are not limited to, those disclosed in the 2005 Pharmacopeia 23NF/28USP in Test Solutions at page 2858 and/or other simulated gastric fluids and simulated intestinal fluids known to those of skill in the art, for example, simulated gastric fluid and/or intestinal fluid prepared without enzymes.

In various aspects, the modified-release formulation can be substantially stable in chyme. For example, there is, in some aspects, a loss of less about 50% or about 40%, or about 30%, or about 20%, or about 10% of the activity or viability of the bacteria in the bacterial mixture in about 10, or 9, or 8, or 7, or 6, or 5, or 4, or 3, or 2, or 1 hour from administration.

In various aspects, the modified-release formulations can be designed for immediate release (e.g. upon ingestion). In various aspects, the modified-release formulations can have sustained-release profiles, i.e. slow release of the active ingredient(s) in the body (e.g., GI tract) over an extended period of time. In various aspects, the modified-release formulations can have a delayed-release profile, i.e. not immediately release the active ingredient(s) upon ingestion; rather, postponement of the release of the active ingredient(s) until the composition is lower in the GI tract; for example, for release in the small intestine (e.g., one or more of duodenum, jejunum, ileum) or the large intestine (e.g., one or more of cecum, ascending, transverse, descending or sigmoid portions of the colon, and rectum). For example, a composition can be enteric coated to delay release of the active ingredient(s) until it reaches the small intestine or large intestine.

In various aspects, the modified-release formulations can utilize one or more modified-release coatings such as delayed-release coatings to provide for effective, delayed yet substantial delivery of the bacterial mixture to the GI tract together with, optionally, additional therapeutic agents.

In an aspect, the delayed-release coating includes an enteric agent that is substantially stable in acidic environments and substantially unstable in near neutral to alkaline environments. In an aspect, the delayed-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethyl ethylcellulose, and EUDRAGIT®-type polymer (poly(methacrylic acid, methylmethacrylate), hydroxypropyl methylcellulose acetate succinate, cellulose acetate trimellitate, shellac or other suitable enteric coating polymers. The EUDRAGIT®-type polymers include, for example, EUDRAGIT® FS 30D, L 30 D-55, L 100-55, L 100, L 12.5, L 12.5 P, RL 30 D, RL PO, RL 100, RL 12.5, RS 30 D, RS PO, RS 100, RS 12.5, NE 30 D, NE 40 D, NM 30 D, S 100, S 12,5, and S 12,5 P. Similar polymers include Kollicoat® MAE 30 DP and Kollicoat® MAE 100 P. In some aspects, one or more of EUDRAGIT® FS 30D, L 30 D-55, L 100-55, L 100, L 12,5, L 12,5 P RL 30 D, RL PO, RL 100, RL 12,5, RS 30 D, RS PO, RS 100, RS 12,5, NE 30 D, NE 40 D, NM 30 D, S 100, S 12,5 S 12,5 P, Kollicoat® MAE 30 DP and Kollicoat® MAE 100 P is used. In various aspects, the enteric agent can be a combination of the foregoing solutions or dispersions.

In certain aspects, one or more coating system additives are used with the enteric agent. For example, one or more PlasACRYL™ additives can be used as an anti-tacking agent coating additive. Illustrative PlasACRYL™ additives include, but are not limited to, PlasACRYL™ HTP20 and PlasACRYL™ T20.

In another aspect, the delayed-release coating can degrade as a function of time when in aqueous solution without regard to the pH and/or presence of enzymes in the solution. Such a coating can comprise a water insoluble polymer. Its solubility in aqueous solution is therefore independent of the pH. The term “pH independent” as used herein means that the water permeability of the polymer and its ability to release pharmaceutical ingredients is not a function of pH and/or is only very slightly dependent on pH. Such coatings can be used to prepare, for example, sustained release formulations. Suitable water insoluble polymers include pharmaceutically acceptable non-toxic polymers that are substantially insoluble in aqueous media, e.g., water, independent of the pH of the solution. Suitable polymers include, but are not limited to, cellulose ethers, cellulose esters, or cellulose ether-esters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like. Other examples of insoluble polymers include, but are not limited to, lacquer, and acrylic and/or methacrylic ester polymers, polymers or copolymers of acrylate or methacrylate having a low quaternary ammonium content, or mixture thereof and the like. Other examples of insoluble polymers include EUDRAGIT RS®, EUDRAGIT RL®, and EUDRAGIT NE®. Insoluble polymers can include polyvinyl esters, polyvinyl acetals, polyacrylic acid esters, butadiene styrene copolymers, and the like. In an aspect, colonic delivery is achieved by use of a slowly eroding wax plug (e.g., various PEGS, including for example, PEG6000).

In a further aspect, the delayed-release coating can be degraded by a microbial enzyme present in the gut flora. In an aspect, the delayed-release coating can be degraded by bacteria present in the small intestine. In another aspect, the delayed-release coating can be degraded by bacteria present in the large intestine.

In various aspects, the modified release formulation can be designed for release in the colon. Various colon-specific delivery approaches can be utilized. For example, the modified release formulation can be formulated using a colon-specific drug delivery system (CODES) as described for example, in Li et al., AAPS PharmSciTech (2002), 3(4): 1-9, the entire contents of which are incorporated herein by reference. Drug release in such a system is triggered by colonic microflora coupled with pH-sensitive polymer coatings. For example, the formulation can be designed as a core tablet with three layers of polymer. The first coating is an acid-soluble polymer (e.g., EUDRAGIT E), the outer coating is enteric, along with a hydroxypropyl methylcellulose barrier layer interposed in between. In another aspect, colon delivery can be achieved by formulating the pharmaceutical composition (e.g., comprising a microbial cocktail) with specific polymers that degrade in the colon such as, for example, pectin. The pectin can be further gelled or crosslinked with a cation such as a zinc cation. In an aspect, the formulation is in the form of ionically crosslinked pectin beads which are further coated with a polymer (e.g., EUDRAGIT polymer). Additional colon specific formulations include, but are not limited to, pressure-controlled drug delivery systems (prepared with, for example, ethylcellulose) and osmotic controlled drug delivery systems (i.e., ORDS-CT).

Formulations for colon specific delivery of the bacterial mixture (and/or additional therapeutic agents), as described herein, can be evaluated using, for example, in vitro dissolution tests. For example, parallel dissolution studies in different buffers can be undertaken to characterize the behavior of the formulations at different pH levels. Alternatively, in vitro enzymatic tests can be carried out. For example, the formulations can be incubated in fermenters containing suitable medium for bacteria, and the amount of drug released at different time intervals is determined. Drug release studies can also be done in buffer medium containing enzymes or rat or guinea pig or rabbit cecal contents and the amount of drug released in a particular time is determined. In a further aspect, in vivo evaluations can be carried out using animal models such as dogs, guinea pigs, rats, and pigs. Further, clinical evaluation of colon specific drug delivery formulations can be evaluated by calculating drug delivery index (DDI) which considers the relative ratio of RCE (relative colonic tissue exposure to the drug) to RSC (relative amount of drug in blood i.e. that is relative systemic exposure to the drug). Higher drug DDI indicates better colon drug delivery. Absorption of drugs from the colon can be monitored by colonoscopy and intubation.

In various aspects, the present formulations provide for substantial uniform delivery of the bacterial mixture (and/or additional therapeutic agent) in the area of release in the GI tract. In an aspect, the present formulations minimize patchy or heterogeneous release of the bacterial mixture.

In various aspects, the present formulations provide for release of multiple doses of one or more bacterial mixtures along the GI tract. For example, the composition and/or formulation can release multiple doses of the same bacterial mixture at different locations along the intestines, at different times, and/or at different pH. Alternatively, the composition and/or formulation can release a dose of different bacterial mixtures at different locations along the intestines, at different times, and/or at a different pH. In an aspect, the pharmaceutical composition comprises a first bacterial mixture comprising one or more bacterial isolates that is released at a first location in the intestine, and a second bacterial mixture comprising a preparation of uncultured fecal bacteria that is released at a second location in the intestine. In an aspect, the first bacterial mixture is released in the ileum, and the second bacterial mixture is released in the colon.

The overall release profile of such a formulation can be adjusted using, for example, multiple particle types or multiple layers. For example, in an aspect, a first bacterial mixture (or first dose of a bacterial mixture) can be formulated for release in, for example, the small intestine (e.g., one or more of duodenum, jejunum, ileum), whereas the second bacterial mixture (or second dose of the bacterial mixture) is formulated for delayed release in, for example, the large intestine (e.g., one or more of cecum, ascending, transverse, descending or sigmoid portions of the colon, and rectum). In another example, the first bacterial mixture (or first dose of a bacterial mixture) can be formulated for release in, for example, the small intestine (e.g., one or more of duodenum, jejunum, ileum), whereas the second bacterial mixture (or second dose of a bacterial mixture) is formulated for delayed release in, for example, another part of the small intestine (e.g., one or more of duodenum, jejunum, ileum). In another aspect, the first bacterial mixture (or first dose of a bacterial mixture) can be formulated for release in, for example, the large intestine (e.g., one or more of cecum, ascending, transverse, descending or sigmoid portions of the colon, and rectum), whereas the second bacterial mixture (or second dose of the bacterial mixture) is formulated for delayed release in, for example, another part of the large intestine (e.g., one or more of cecum, ascending, transverse, descending or sigmoid portions of the colon, and rectum). In various aspects, the composition and/or formulation can release at least one dose, at least two doses, at least three doses, at least four doses, or at least five doses of the bacterial mixture at different locations along the intestines, at different times, and/or at different pH. Likewise, in various aspects, the composition and/or formulation can release at least one bacterial mixture, at least two bacterial mixtures, at least three bacterial mixtures, at least four bacterial mixtures, or at least five bacterial mixtures at different locations along the intestines, at different times, and/or at different pH.

In another aspect, a delayed or gradual enteric release formulation comprises the use of a bilayer tablet or capsule which comprises a first layer comprising a polyalkylene oxide, a polyvinylpyrrolidone, a lubricant, or a mixture thereof, and a second osmotic push layer comprising polyethylene oxide, carboxy-methylcellulose, or both. In an aspect, a delayed or gradual enteric release formulation comprises the use of a release-retarding matrix material selected from the group consisting of an acrylic polymer, a cellulose, a wax, a fatty acid, shellac, zein, hydrogenated vegetable oil, hydrogenated castor oil, polyvinylpyrrolidine, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a co-polymer of lactic and glycolic acid, carboxymethyl starch, potassium methacrylate/divinylbenzene copolymer, crosslinked polyvinylpyrrolidone, poly inylalcohols, polyvinylalcohol copolymers, polyethylene glycols, non-crosslinked polyvinylpyrrolidone, polyvinylacetates, polyvinylacetate copolymers, or any combination thereof. In an aspect, a delayed or gradual enteric release formulation comprises the use of a microenvironment pH modifier.

It will be understood that a pharmaceutical composition described herein can comprise multiple distinct bacterial mixtures, for example to achieve different delivery location profiles for each bacterial mixture. In an aspect, a pharmaceutical composition comprises at least two bacterial mixtures, such that a first bacterial mixture comprises one or more bacterial isolates and a second bacterial mixture comprises a preparation of uncultured fecal bacteria. In an aspect, the second bacterial mixture further comprises one or more bacterial isolates that are different than the bacterial isolate(s) in the first bacterial mixture. Alternatively, the second bacterial mixture can consist essentially of the preparation of uncultured fecal bacteria. In another aspect, the first bacterial mixture can comprise only one bacterial isolate. A pharmaceutical composition can comprise any number of bacterial mixtures, for example one, two, three, four, five, six, seven, eight, nine, ten, or more than ten bacterial mixtures that each contain a different bacterial isolate, a different combination of bacterial isolates, a preparation of uncultured fecal bacteria, or a different combination of uncultured fecal bacteria with one or more bacterial isolates.

In an aspect, a pharmaceutical composition can be a drench. In one aspect, a drench is prepared by choosing a saline-suspended form of a pharmaceutical composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition many substances, which affect the hydrophilicity, density and surface tension of the liquid, can assist in making suspensions in individual cases. For example, silicone anti-foams, glycols, sorbitol, and sugars can be useful suspending agents.

In some aspects, one or more bacterial isolates described herein are in the form of live, vegetative cells. In some aspects, one or more bacterial isolates described herein are in the form of spores. In some aspects, one or more bacterial isolates described herein are lyophilized. By way of non-limiting example, lyophilization can be via methods known in the art, including those described in U.S. Pat. No. 7,799,328, the contents of which are hereby incorporated by reference in their entirety. In some aspects, lyophilized bacterial mixtures described herein are placed in an enterically coated soft gel or capsule.

In various aspects, formulations can take the form of those described in one or more of U.S. Pat. Nos. 8,535,713 and 8,9117,77 and US Patent Publication Nos. 20120141585, 20120141531, 2006/001896, 2007/0292523, 2008/0020018, 2008/0113031, 2010/0203120, 2010/0255087, 2010/0297221, 2011/0052645, 2013/0243873, 2013/0330411, 2014/0017313, and 2014/0234418, the contents of which are hereby incorporated by reference in their entirety.

In various aspects, formulations can take the form of those as described in International Patent Publication No. WO 2008/135090, the contents of which are hereby incorporated by reference in their entirety.

In various aspects, formulations can take the form of those described in one or more of U.S. Pat. Nos. 4,196,564; 4,196,565; 4,247,006; 4,250,997; 4,268,265; 5,317,849; 6,572,892; 7,712,634; 8,074,835; 8,398,912; 8,440,224; 8,557,294; 8,646,591; 8,739,812; 8,810,259; 8,852,631; and 8,911,788 and US Patent Publication Nos. 2014/0302132; 2014/0227357; 20140088202; 20130287842; 2013/0295188; 2013/0307962; and 20130184290, the contents of which are hereby incorporated by reference in their entirety.

Administration and Dosage

It will be appreciated that the dose of a pharmaceutical composition or the bacterial cells therein (e.g., a bacterial mixture comprising one or more bacterial isolates and/or a preparation of uncultured fecal bacteria) will vary according to, for example, the particular dosage form, the mode of administration to a subject, the identity of a bacterial isolate, if any, in the composition, the number of bacterial isolates, if any, in the composition. These factors, as well as variables that may modify the activity of the bacteria in a bacterial mixture (e.g., subject body weight, sex and diet, time of administration, route of administration, rate of excretion, condition of the subject, drug combinations, genetic disposition and reaction sensitivities) can be taken into account by those skilled in the art to generate an effective dose or dosage regime for treatment or prevention of at least one symptom of a disorder described herein (e.g., a disorder associated with a gut dysbiosis that can be treated or prevented by administration of at least one SCFA). Administration can be carried out continuously or in one or more discrete doses within the maximum tolerated dose. Optimal administration rates for a given set of conditions can be ascertained by those skilled in the art using conventional dosage administration tests.

In various aspects, the dose of the pharmaceutical composition or the bacterial cells therein (e.g., a bacterial mixture comprising one or more bacterial isolates and/or a preparation of uncultured fecal bacteria) is effective to modulate a patient's microbiome to favor an ecological balance, so as to treat or prevent one or more symptoms of a disorder associated with a gut dysbiosis (e.g., by delivering one or more SCFAs to the intestine of the patient).

In one aspect, a pharmaceutically active or therapeutically effective dose of a bacterial isolate administered to a subject (i.e., in single or multiple administrations) to treat at least one symptom of a disorder (e.g., a disorder associated with a gut dysbiosis that can be treated or prevented by administration of at least one SCFA) comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ CFUs of the bacterial isolate. In another aspect, a pharmaceutically active or therapeutically effective dose of a bacterial isolate administered to a subject (i.e., in single or multiple administrations) to treat at least one symptom of a disorder (e.g., a disorder associated with a gut dysbiosis that can be treated or prevented by administration of at least one SCFA) comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ CFUs of the bacterial isolate. In a further aspect, a pharmacologically active or therapeutically effective dose of a bacterial isolate administered to a subject (i.e., in single or multiple administrations) to treat at least one symptom of a disorder (e.g., a disorder associated with a gut dysbiosis that can be treated or prevented by administration of at least one SCFA) is selected from the group consisting of: from 10⁸ CFUs to 10¹⁴ CFUs, from 10⁹ CFUs to 10¹³ CFUs, from 10¹⁰ CFUs to 10¹² CFUs, from 10¹⁰ CFUs to 10¹¹ CFUs, from 10⁹ CFUs to 10¹⁴ CFUs, from 10⁹ CFUs to 10¹² CFUs, from 10⁹ CFUs to 10¹¹ CFUs, from 10⁹ CFUs to 10¹⁰ CFUs, from 10¹⁰ CFUs to 10¹⁴ CFUs, from 10¹⁰ CFUs to 10¹³ CFUs, from 10¹¹ CFUs to 10¹⁴ CFUs, from 10¹¹ CFUs to 10¹³ CFUs, from 10¹² CFUs to 10¹⁴ CFUs, and from 10¹³ CFUs to 10¹⁴ CFUs of the bacterial isolate.

In an aspect, a pharmaceutical composition comprises one or more bacterial isolates, with each bacterial isolate present in each unit dose at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutically effective dose of a bacterial isolate administered to a subject (i.e., in single or multiple administrations) to treat at least one symptom of a disorder comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores of the bacterial isolate. In another aspect, a pharmaceutically active or therapeutically effective dose of a bacterial isolate administered to a subject i.e. in single or multiple administrations) to treat at least one symptom of a disorder comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ total cells or spores of the bacterial isolate. In a further aspect, a pharmacologically active or therapeutically effective dose of a bacterial isolate administered to a subject (i.e., in single or multiple administrations) to treat at least one symptom of a disorder (e.g., a disorder associated with a gut dysbiosis that can be treated or prevented by administration of at least one SCFA) is selected from the group consisting of: from 10⁸ to 10¹⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores of the bacterial isolate.

In an aspect, the pharmaceutically active or therapeutically effective dose cell count of a bacterial isolate is directed to live cells. In one aspect, a pharmaceutical composition comprises one or more bacterial isolates, with each bacterial isolates present in each dosage unit at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In an aspect, a pharmaceutical composition described herein is in the form of a capsule, and each capsule comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores of a bacterial isolate. In an aspect, a pharmaceutical composition described herein is in the form of a capsule, and each capsule comprises from 10⁸ to 10¹⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, or from 10¹³ to 10¹⁴ cells or spores of a bacterial isolate.

In one aspect, a pharmaceutically active or therapeutically effective dose of a preparation of uncultured fecal bacteria administered to a subject (i.e., in single or multiple administrations) to treat at least one symptom of a disorder (e.g., associated with a gut dysbiosis) comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ CFUs of the preparation of uncultured fecal bacteria. In another aspect, a pharmaceutically active or therapeutically effective dose of a preparation of uncultured fecal bacteria administered to a subject (i.e., in single or multiple administrations) to treat at least one symptom of a disorder (e.g., associated with a gut dysbiosis) comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ CFUs of the preparation of uncultured fecal bacteria. In a further aspect, a pharmacologically active or therapeutically effective dose of a preparation of uncultured fecal bacteria administered to a subject (i.e., in single or multiple administrations) to treat at least one symptom of a disorder (e.g., associated with a gut dysbiosis) is selected from the group consisting of: from 10⁸ CFUs to 10¹⁴ CFUs, from 10⁹ CFUs to 10¹³ CFUs, from 10¹⁰ CFUs to 10¹² CFUs, from 10¹⁰ CFUs to 10¹¹ CFUs, from 10⁹ CFUs to 10¹⁴ CFUs, from 10⁹ CFUs to 10¹² CFUs, from 10⁹ CFUs to 10¹¹ CFUs, from 10⁹ CFUs to 10¹⁰ CFUs, from 10¹⁰ CFUs to 10¹⁴ CFUs, from 10¹⁰ CFUs to 10¹³ CFUs, from 10¹¹ CFUs to 10¹⁴ CFUs, from 10¹¹ CFUs to 10¹³ CFUs, from 10¹² CFUs to 10¹⁴ CFUs, and from 10¹³ CFUs to 10¹⁴ CFUs of the preparation of uncultured fecal bacteria.

In an aspect, uncultured fecal bacteria are present in each unit dose of a pharmaceutical composition at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutically effective dose of a preparation of uncultured fecal bacteria administered to a subject (i.e., in single or multiple administrations) to treat at least one symptom of a disorder (e.g., associated with a gut dysbiosis) comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores of the preparation of uncultured fecal bacteria. In another aspect, a pharmaceutically active or therapeutically effective dose of a preparation of uncultured fecal bacteria administered to a subject i.e. in single or multiple administrations) to treat at least one symptom of a disorder (e.g., associated with a gut dysbiosis) comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ total cells or spores of the preparation of uncultured fecal bacteria. In a further aspect, a pharmacologically active or therapeutically effective dose of a preparation of uncultured fecal bacteria administered to a subject (i.e., in single or multiple administrations) to treat at least one symptom of a disorder (e.g., associated with a gut dysbiosis) is selected from the group consisting of: from 10⁸ to 10¹⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores of the preparation of uncultured fecal bacteria.

In an aspect, the pharmaceutically active or therapeutically effective dose cell count of a preparation of uncultured fecal bacteria is directed to live cells. In one aspect, a preparation of uncultured fecal bacteria is present in each unit dose of a pharmaceutical composition at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In an aspect, a pharmaceutical composition described herein is in the form of a capsule, and each capsule comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores of a preparation of uncultured fecal bacteria. In an aspect, a pharmaceutical composition described herein is in the form of a capsule, and each capsule comprises from 10⁸ to 10¹⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, or from 10¹³ to 10¹⁴ cells or spores of a preparation of uncultured fecal bacteria.

A subject can be administered one or more bacterial isolates (e.g., comprising one or more SCFA-producing bacterial strains) combined with a preparation of uncultured fecal bacteria for treatment of one or more symptoms of a disorder (e.g., a disorder associated with a gut dysbiosis that can be treated or prevented by administration of at least one SCFA). In such cases, the bacterial isolate(s) and preparation of uncultured fecal bacteria can be administered to the subject together in the same pharmaceutical composition, or in separate compositions. Further, a pharmaceutical composition (e.g., comprising one or more bacterial isolates, a preparation of uncultured fecal bacteria, or both) can be administered to the subject in a single unit dose or multiple unit doses, for example as part of a dosage regime. In an aspect, the dosage of the preparation of uncultured fecal bacteria (e.g. measured by CFU or cell/spore count) administered to a subject is greater than the dosage of the bacterial isolate. Alternatively, the dosage of the preparation of uncultured fecal bacteria (e.g. measured by CFU or cell/spore count) administered to the subject can be less than the dosage of the bacterial isolate. In another aspect, the dosage of the preparation of uncultured fecal bacteria (e.g. measured by CFU or cell/spore count) can be about the same as the dosage of the bacterial isolate. For example, in an aspect a subject can be administered a bacterial isolate (e.g. comprising one or more SCFA-producing bacterial strains) at a dosage of about 10¹⁰ cells and a preparation of uncultured fecal bacteria at a dosage of about 10¹⁰ cells to treat or prevent one or more symptoms of a disorder described herein.

In an aspect, the number of cells of a bacterial isolate (e.g., comprising an SCFA-producing bacterial strain) administered to a subject to treat one or more symptoms of a disorder described herein (e.g., a disorder associated with a gut dysbiosis that can be treated or prevented by administration of at least one SCFA) is about the same or greater than the total number of cells of a preparation of uncultured fecal bacteria administered to the subject. Alternatively, the number of cells of a bacterial isolate administered to a subject to treat one or more symptoms of a disorder (e.g., a disorder associated with a gut dysbiosis that can be treated or prevented by administration of at least one SCFA) can be about the same or less than the total number of cells of a preparation of uncultured fecal bacteria administered to the subject.

In an aspect, a pharmaceutical composition comprises a bacterial mixture that comprises multiple bacterial isolates (e.g., at least one of which comprises one or more SCFA-producing bacterial strains). In another aspect, at least two bacterial isolates are present at about the same amount or dosage (e.g., about the same number of viable cells or spores, or about the same CFUs). In another aspect, at least three bacterial isolates, at least four bacterial isolates, at least five bacterial isolates, at least six bacterial isolates, at least seven bacterial isolates, at least eight bacterial isolates, at least nine bacterial isolates, at least ten bacterial isolates, or more than ten bacterial isolates are present in the pharmaceutical composition at about the same amount or dosage (e.g., about the same number of viable cells or spores, or about the same CFUs). In another aspect, all of the bacterial isolates in a bacterial mixture are present in about the same amounts.

In an aspect, a pharmaceutical composition comprises a bacterial mixture comprising multiple bacterial isolates, and at least two of the multiple bacterial isolates are present at different amounts or dosages (e.g., different numbers of viable cells or spores, or different CFUs). In another aspect, at least three, at least four, at least five, at least six, at least seven, at least eight, at least nine, at least ten, or more than ten bacterial isolates are present in the bacterial mixture at different amounts or dosages.

A pharmaceutical composition can comprise a bacterial mixture comprising multiple bacterial isolates (e.g., at least one of which comprising an SCFA-producing bacterial strain) in combination with a preparation of uncultured fecal bacteria. In an aspect, each bacterial isolate is present in the composition at an amount or dosage that is greater than the amount or dosage of the preparation of uncultured fecal bacteria (e.g., measured as numbers of viable cells or spores, or CFUs). In another aspect, each bacterial isolate is present in the composition at an amount or dosage that is less than the amount or dosage of the preparation of uncultured fecal bacteria (e.g., measured as numbers of viable cells or spores, or CFUs). In another aspect, at least one bacterial isolate is present in the composition at an amount or dosage that is greater than the amount or dosage of the preparation of uncultured fecal bacteria, and at least one bacterial isolate is present in the composition at an amount or dosage that is less than the amount or dosage of the preparation of uncultured fecal bacteria (e.g., measured as numbers of viable cells or spores, or CFUs).

In an aspect, a pharmaceutical composition comprises one or more bacterial isolates at an amount or dosage which is at or above the minimum amount or dosage of the bacterial isolate required to be administered to a subject for engraftment of the bacterial isolate to occur in the intestine of the subject. For example, a minimum dosage of the bacterial isolate required for engraftment of the bacterial isolate into the intestine of the subject can be at least 10⁶ cells, at least 10⁷ cells, at least 10⁸ cells, at least 10⁹ cells, at least 10¹⁰ cells, at least 10¹¹ cells, or at least 10¹² cells. In an aspect a first and second bacterial isolate of a microbial cocktail engraft in the intestine of a subject at different minimal dosages or amounts, and a dosage or amount of each of the first and second bacterial isolate in the microbial cocktail varies corresponding to the respective minimal dosage or amount required for engraftment of the respective bacterial isolate.

Individual doses of the pharmaceutical composition (e.g., comprising a bacterial mixture) can be administered in unit dosage forms (e.g., tablets or capsules) containing, for example, from about 0.01 mg to about 5,000 mg, from about 0.01 mg to about 4,000 mg, from about 0.01 mg to about 3,000 mg, from about 0.01 mg to about 2,000 mg, from about 0.01 mg to about 1,000 mg, from about 0.01 mg to about 950 mg, from about 0.01 mg to about 900 mg, from about 0.01 mg to about 850 mg, from about 0.01 mg to about 800 mg, from about 0.01 mg to about 750 mg, from about 0.01 mg to about 700 mg, from about 0.01 mg to about 650 mg, from about 0.01 mg to about 600 mg, from about 0.01 mg to about 550 mg, from about 0.01 mg to about 500 mg, from about 0.01 mg to about 450 mg, from about 0.01 mg to about 400 mg, from about 0.01 mg to about 350 mg, from about 0.01 mg to about 300 mg, from about 0.01 mg to about 250 mg, from about 0.01 mg to about 200 mg, from about 0.01 mg to about 150 mg, from about 0.01 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg, from about 0.1 mg to about 30 mg, from about 0.1 mg to about 20 mg, from about 0.1 mg to about 10 mg, from about 0.1 mg to about 5 mg, from about 0.1 mg to about 3 mg, from about 0.1 mg to about 1 mg of the active ingredient per unit dosage form, or from about 5 mg to about 80 mg per unit dosage form. For example, a unit dosage form can include about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 2,000 mg, about 3,000 mg, about 4,000 mg, or about 5,000 mg of the active ingredient, inclusive of all values and ranges therebetween.

In an aspect, the pharmaceutical composition (e.g., comprising a bacterial mixture) is administered at an amount of from about 0.01 mg to about 100 mg daily, an amount of from about 0.01 mg to about 5,000 mg daily, about 0.01 mg to about 4,000 mg daily, about 0.01 mg to about 3,000 mg daily, about 0.01 mg to about 2,000 mg daily, about 0.01 mg to about 1,000 mg daily, from about 0.01 mg to about 950 mg daily, from about 0.01 mg to about 900 mg daily, from about 0.01 mg to about 850 mg daily, from about 0.01 mg to about 800 mg daily, from about 0.01 mg to about 750 mg daily, from about 0.01 mg to about 700 mg daily, from about 0.01 mg to about 650 mg daily, from about 0.01 mg to about 600 mg daily, from about 0.01 mg to about 550 mg daily, from about 0.01 mg to about 500 mg daily, from about 0.01 mg to about 450 mg daily, from about 0.01 mg to about 400 mg daily, from about 0.01 mg to about 350 mg daily, from about 0.01 mg to about 300 mg daily, from about 0.01 mg to about 250 mg daily, from about 0.01 mg to about 200 mg daily, from about 0.01 mg to about 150 mg daily, from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily. In various aspects, the bacterial mixture (and/or additional therapeutic agents) is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 2,000 mg, about 3,000 mg, about 4,000 mg, or about 5,000 mg inclusive of all values and ranges therebetween.

In some aspects, a suitable dosage of the pharmaceutical composition (e.g., comprising a bacterial mixture) is in a range of about 0.01 mg/kg to about 100 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, about 20 mg/kg body weight, about 30 mg/kg body weight, about 40 mg/kg body weight, about 50 mg/kg body weight, about 60 mg/kg body weight, about 70 mg/kg body weight, about 80 mg/kg body weight, about 90 mg/kg body weight, or about 100 mg/kg body weight, inclusive of all values and ranges therebetween. In other aspects, a suitable dosage of the composition in a range of about 0.01 mg/kg to about 100 mg/kg of body weight, in a range of about 0.01 mg/kg to about 90 mg/kg of body weight, in a range of about 0.01 mg/kg to about 80 mg/kg of body weight, in a range of about 0.01 mg/kg to about 70 mg/kg of body weight, in a range of about 0.01 mg/kg to about 60 mg/kg of body weight, in a range of about 0.01 mg/kg to about 50 mg/kg of body weight, in a range of about 0.01 mg/kg to about 40 mg/kg of body weight, in a range of about 0.01 mg/kg to about 30 mg/kg of body weight, in a range of about 0.01 mg/kg to about 20 mg/kg of body weight, in a range of about 0.01 mg/kg to about 10 mg/kg of body weight, in a range of about 0.01 mg/kg to about 9 mg/kg of body weight, in a range of about 0.01 mg/kg to about 8 mg/kg of body weight, in a range of about 0.01 mg/kg to about 7 mg/kg of body weight, in a range of 0.01 mg/kg to about 6 mg/kg of body weight, in a range of about 0.05 mg/kg to about 5 mg/kg of body weight, in a range of about 0.05 mg/kg to about 4 mg/kg of body weight, in a range of about 0.05 mg/kg to about 3 mg/kg of body weight, in a range of about 0.05 mg/kg to about 2 mg/kg of body weight, in a range of about 0.05 mg/kg to about 1.5 mg/kg of body weight, or in a range of about 0.05 mg/kg to about 1 mg/kg of body weight.

In accordance with certain aspects, the pharmaceutical composition (e.g., comprising a bacterial mixture) can be administered, for example, more than once daily, about once per day, about every other day, about every third day, about once a week, about once every two weeks, about once every month, about once every two months, about once every three months, about once every six months, or about once every year.

In an aspect, a pharmaceutical composition can be administered to a patient in need thereof at least once daily for at least two consecutive days. In another aspect, a pharmaceutical composition is administered at least once daily for at least 3, 4, 5, 6, 7, 8, 9, 10, 11, 12, 13, 14, or 15 consecutive days. In another aspect, a pharmaceutical composition is administered at least once daily for at least 1, 2, 3, 4, 5, 6, 7, 8, 9, 10, 11, or 12 consecutive weeks. In another aspect, a pharmaceutical composition is administered at least twice, three times, four times, or five times per week for at least 1, 2, 3, 4, 5, 6, 7, 8, 9, 10, 11, or 12 consecutive weeks. In another aspect, a pharmaceutical composition is administered at least once daily for at most 4, 5, 6, 7, 8, 9, 10, 11, 12, 13, 14, 15, 16, 17, 18, 19, or 20 consecutive days or weeks. In a further aspect, a pharmaceutical composition is administered at least once daily for at most 1, 2, 3, 4, 5, 6, 7, 8, 9, 10, 11, or 12 consecutive weeks or months. In yet another aspect, a pharmaceutical composition is administered at least once for at least 1, 2, 3, 4, 5, 6, 7, 8, 9, 10, 11, or 12 consecutive months or years, chronically for a subject's entire life span, or an indefinite period of time.

In an aspect, a pharmaceutical composition can be administered to a patient in need thereof at least twice daily for at least two consecutive days. In an aspect, a pharmaceutical composition is administered at least twice daily for at least 3, 4, 5, 6, 7, 8, 9, 10, 11, 12, 13, 14, or 15 consecutive days. In another aspect, a pharmaceutical composition is administered at least twice daily for at least 1, 2, 3, 4, 5, 6, 7, 8, 9, 10, 11, or 12 consecutive weeks. In another aspect, a pharmaceutical composition is administered at least twice daily for at most 4, 5, 6, 7, 8, 9, 10, 11, 12, 13, 14, 15, 16, 17, 18, 19, or 20 consecutive days or week. In another aspect, a pharmaceutical composition is administered at least twice daily for at most 1, 2, 3, 4, 5, 6, 7, 8, 9, 10, 11, or 12 consecutive weeks or months. In another aspect, a pharmaceutical composition is administered at least twice for at least 1, 2, 3, 4, 5, 6, 7, 8, 9, 10, 11, or 12 consecutive months or years, chronically for a subject's entire life span, or an indefinite period of time.

In an aspect of the present disclosure, a pharmaceutical composition can be administered to a patient in need thereof at least three times daily for at least two consecutive days. In an aspect, a pharmaceutical composition is administered at least three times daily for at least 3, 4, 5, 6, 7, 8, 9, 10, 11, 12, 13, 14, or 15 consecutive days. In an aspect, a pharmaceutical composition is administered at least three times daily for at least 1, 2, 3, 4, 5, 6, 7, 8, 9, 10, 11, or 12 consecutive weeks. In an aspect, a pharmaceutical composition is administered at least three times daily for at most 4, 5, 6, 7, 8, 9, 10, 11, 12, 13, 14, 15, 16, 17, 18, 19, or 20 consecutive days or weeks. In an aspect, a pharmaceutical composition is administered at least three times daily for at most 1, 2, 3, 4, 5, 6, 7, 8, 9, 10, 11, or 12 consecutive weeks or months. In an aspect, a pharmaceutical composition is administered at least three times for at least 1, 2, 3, 4, 5, 6, 7, 8, 9, 10, 11, or 12 consecutive months or years, chronically for a subject's entire life span, or an indefinite period of time.

In an aspect, a pharmaceutical composition can be administered to a patient in need thereof at a dosing schedule of at least once or twice daily for at least three consecutive days or weeks. In an aspect, a dose is administered at least once, twice, or three times daily for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In an aspect, a pharmaceutical composition can be administered to a patient in need thereof at a dosing schedule of once-a-week, twice-a-week, or thrice-a-week. The term “once-a-week” means that a dose is administered typically only once in a week, for example, on the same day of each week. “Twice-a-week” means that a dose is administered typically only two times in a week, for example, on the same two days of each weekly period. “Thrice-a-week” means that a dose is administered typically only three times in a week, for example, on the same three days of each weekly period.

In an aspect, a pharmaceutical composition can be administered to a patient in need thereof, wherein the administration comprises a first dosing schedule followed by a second dosing schedule. In an aspect, a first dosing schedule comprises a treatment or induction dose. In an aspect, a second dosing schedule comprises a maintenance dose. For example, a pharmaceutically active maintenance dose of a second dosage schedule can be lower than or equal to a pharmaceutically active induction dose of a first dosing schedule. In other examples, a maintenance dose of a second dosing schedule can be higher than an induction dose of a first dosing schedule.

At least one of a first and second dosing schedule for administering a pharmaceutical composition can comprise administration of the composition at least once daily for at least one day. In an aspect, at least one of a first or second dosing schedule comprises administration of the composition at least once daily for at least 2, 3, 4, 5, 6, 7, 8, 9, 10, 11, 12, 13, 14, or 15 consecutive days. In an aspect, at least one of a first or second dosing schedule comprises administration of the composition at least once daily for at least 1, 2, 3, 4, 5, 6, 7, 8, 9, 10, 11, or 12 consecutive weeks. In an aspect, at least one of a first or second dosing schedule comprises administration of the composition for at most 4, 5, 6, 7, 8, 9, 10, 11, 12, 13, 14, 15, 16, 17, 18, 19, or 20 consecutive days or weeks. In an aspect, at least one of a first or second dosing schedule comprises administration of the composition for at most 1, 2, 3, 4, 5, 6, 7, 8, 9, 10, 11, or 12 consecutive weeks or months. In an aspect, at least one of a first or second dosing schedule comprises administration of the composition for at least 1, 2, 3, 4, 5, 6, 7, 8, 9, 10, 11, or 12 consecutive months or years, chronically for a subject's entire life span, or an indefinite period of time.

In an aspect, at least one of a first or second dosing schedule used in a method can be once-a-week, twice-a-week, or thrice-a-week.

In an aspect, at least one of a first and second dosing schedule can last for at least about 2, 4, 6, 8, 10, 12, 18, 24, 36, 48, 72, or 96 months. In an aspect, a second dosing schedule lasts permanently, for a treated subject's entire life span, or an indefinite period of time. In an aspect, at least one of a first and second dosing schedule is a continuous dosing schedule. In an aspect, at least one of a first and second dosing schedule is an intermittent dosing schedule. In an aspect, at least one of a first and second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In an aspect, at least one of a first and second dosing schedule comprises administering a dose every other day, every two days, or every 3, 4, 5, 6, 7, 8 days. In an aspect, a dose is administered for an extended period of time with or without titration (or otherwise changing the dosage or dosing schedule).

In an aspect, the interval between a first and a second dosing schedule is at least about 1, 2, 3, 4, 5, 6, or 7 days, or at least about 1, 2, 3, 4, 5, 6, 7, 8, 9, 10, 11, or 12 weeks, or at least about 1, 2, 3, 4, 6, 7, 8, 9, 10, 11, or 12 months.

In an aspect, a second dosing schedule (e.g., a maintenance dose) comprises a dosage about 2, 3, 4, 5, 6, 7, 8, 9, 10, 20, 30, 40, 50, 75, 100, 200, 400, 800, 1000, 5000 or more fold lower than the dosage used in a first dosing schedule (e.g., an initial induction dose). In another aspect, a second dosing schedule (e.g., a maintenance dosing schedule) has an equal or lower dosing frequency than a first dosing schedule (e.g., an initial treatment dosing schedule). In an aspect, a second dosing schedule (e.g., a maintenance dosing schedule) has a higher dosing interval than a first dosing schedule (e.g., an initial treatment dosing schedule).

In various aspects, methods described herein are useful in treatment of a human subject. In some aspects, the human is a pediatric human. In other aspects, the human is an adult human. In other aspects, the human is a geriatric human. In other aspects, the human may be referred to as a patient. In some aspects, the human is a female. In some aspects, the human is a male.

In certain aspects, the human has an age in a range of from about 1 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In one aspect, a subject being treated is a human patient. In one aspect, a patient is a male patient. In one aspect, a patient is a female patient. In one aspect, a patient is a premature newborn. In one aspect, a patient is a term newborn. In one aspect, a patient is a neonate. In one aspect, a patient is an infant. In one aspect, a patient is a toddler. In one aspect, a patient is a young child. In one aspect, a patient is a child. In one aspect, a patient is an adolescent. In one aspect, a patient is a pediatric patient. In one aspect, a patient is a geriatric patient. In one aspect, a human patient is a child patient below about 18, 15, 12, 10, 8, 6, 4, 3, 2, or 1-year-old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old patient (>85 years).

Additional Therapeutic Agents and Co-Formulation

The pharmaceutical compositions described herein can include one or more therapeutic agents in addition to a bacterial mixture, which can be administered to a subject in need thereof in a method described herein. The additional therapeutic agent can be administered simultaneous or sequential with a bacterial mixture (e.g., comprising one or more bacterial isolates and/or a preparation of uncultured fecal bacteria) described herein. Further, the present compositions and formulations can comprise the additional therapeutic agent (e.g. via co-formulation). For example, the additional therapeutic agent, one or more bacterial isolates, and preparation of uncultured fecal bacteria can be combined into a single formulation.

In an aspect, the additional therapeutic agent and bacterial mixture are administered to a subject simultaneously. The term “simultaneously” as used herein, means that the additional therapeutic agent and the bacterial mixture are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the bacterial mixture can be by simultaneous administration of a single formulation (e.g., a formulation comprising the additional therapeutic agent and a bacterial mixture) or of separate formulations (e.g., a first formulation including the additional therapeutic agent and a second formulation including the bacterial mixture).

Co-administration does not require an additional therapeutic agent to be administered simultaneously, if the timing of its administration is such that the pharmacological activities of the additional therapeutic agent and the bacterial mixture (e.g., comprising one or more bacterial isolates and/or a preparation of uncultured fecal bacteria) overlap in time. For example, the additional therapeutic agent and the bacterial mixture can be administered sequentially. The term “sequentially” as used herein means that the additional therapeutic agent and the bacterial mixture are administered with a time separation of more than about 60 minutes. For example, the time between the sequential administration of the additional therapeutic agent and the bacterial mixture can be more than about 60 minutes, more than about 2 hours, more than about 5 hours, more than about 10 hours, more than about 1 day, more than about 2 days, more than about 3 days, or more than about 1 week apart. The optimal administration times will depend on the rates of metabolism, excretion, and/or the pharmacodynamic activity of the additional therapeutic agent and the bacterial mixture being administered. Either of the additional therapeutic agent or the bacterial mixture can be administered first.

In a further aspect, the additional therapeutic agent and the bacterial mixture can be administered to a subject simultaneously but the release of additional therapeutic agent and the bacterial mixture from their respective dosage forms (or single unit dosage form if co-formulated) in the GI tract can occur sequentially.

Co-administration also does not require multiple additional therapeutic agents to be administered to the subject by the same route of administration as a bacterial mixture. Rather, each additional therapeutic agent can be administered by any appropriate route, for example, parenterally or non-parenterally.

In some aspects, the additional therapeutic agent is an agent used to treat or prevent one or more symptoms of a disorder described herein (e.g., a disorder associated with a gut dysbiosis that can be treated or prevented by administration of at least one SCFA). In some aspects, the additional therapeutic agent is selected from the group consisting of isperidone, fluoxetine, aripiprazole, vitamin D, levocarnitine, and a combination thereof.

In some aspects, the additional therapeutic agent is an anti-inflammatory agent such as steroidal anti-inflammatory agents or non-steroidal anti-inflammatory agents (NSAIDS). Steroids, particularly the adrenal corticosteroids and their synthetic analogues, are well known in the art. Non-limiting examples of corticosteroids that can be administered to a subject as an additional therapeutic agent include hydroxyltriamcinolone, alpha-methyl dexamethasone, beta-methyl betamethasone, beclomethasone dipropionate, betamethasone benzoate, betamethasone dipropionate, betamethasone valerate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, clocortelone, clescinolone, dichlorisone, difluprednate, flucloronide, flunisolide, fluoromethalone, fluperolone, fluprednisolone, hydrocortisone, meprednisone, paramethasone, prednisolone, prednisone, beclomethasone dipropionate. (NSAIDS) that can be used, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone, indomethacin, and a combination thereof. Additional anti-inflammatory agents are described, for example, in U.S. Pat. No. 4,537,776, the entire contents of which is incorporated by reference herein.

In some aspects, an additional therapeutic agent that can be incorporated into a pharmaceutical composition is a prebiotic. A prebiotic is a compound or compounds (e.g. comprising one or more nutrients) administered to a subject to promote the growth, proliferation, or activity of one or more microorganisms (e.g., bacteria) in the intestine of the subject (e.g., by providing a substrate to be metabolized by the one or more microorganisms). Without wishing to be bound by theory, prebiotics can be added to a pharmaceutical composition to nutritionally supplement bacteria in the endogenous microbiome of the subject and/or in the pharmaceutical composition itself, e.g., to stimulate the growth or activity of one or more strains of a preparation of uncultured fecal bacteria and/or one or more bacterial isolates. Additionally, one or more prebiotics can be added to a composition to buffer against “shock” to bacteria cells when transitioning those cells to a new environment, for example, subsequent to the isolation and/or purification of a preparation of uncultured fecal bacteria, or before or after freezing, freeze-drying, spray-drying, reconstitution in solution and the like.

Non-limiting examples of prebiotics that can be added to a pharmaceutical composition include an amino acid (e.g., valine, leucine, isoleucine), lactic acid, ammonium nitrate, amylose, barley mulch, biotin, carbonate, cellulose, chitin, choline, fructooligosaccharides (FOSs), fructose, glucose, glycerol, heteropolysaccharide, histidine, homopolysaccharide, hydroxyapatite, inulin, isomaltulose, lactose, lactulose, maltodextrins, maltose, nitrogen, oligodextrose, oligofructose, oligofructose-enriched inulin, an oligosaccharide (e.g. comprising a galactooligosaccharide (GOS), trans-galactooligosaccharide, fructooligosaccharide (FOS), xylooligosaccharides (XOS), mannooligosaccharide, or chitooligosaccharide), pectin, phosphate salts, phosphorus, polydextroses, polyols, potash, potassium, sodium nitrate, starch, sucrose, sulfur, sun fiber, tagatose, thiamine, trehalose, vitamins, a water-soluble carbohydrate, a fermentable polysaccharide, a dietary fiber, resistant starch, barley, white navy bean powder, and a combination thereof. Illustrative prebiotics include complex carbohydrates, amino acids, peptides, or other essential nutritional components for the survival of the bacterial composition.

In an aspect, a subject is not pretreated with a prebiotic prior to treatment with a pharmaceutical composition. In another aspect, the pharmaceutical composition is not supplemented with a prebiotic.

In an aspect, a prebiotic can be included (e.g., in dry or liquid forms) in a pharmaceutical composition described herein, for example, comprising a bacterial mixture.

Alternately, or additionally, a prebiotic to be administered to a subject can be included (e.g., in dry or liquid forms) in a distinct pharmaceutical composition lacking a bacterial mixture.

A prebiotic can be administered to a subject before, contemporaneously with, and/or after administration of a pharmaceutical composition comprising a bacterial mixture, either in the same pharmaceutical composition or in a separate pharmaceutical composition.

A prebiotic can be provided and administered in a single dose or in multiple doses. When provided as a single dose, a single composition can comprise only one prebiotic or a mixture of prebiotics. When provided in multiple doses, each composition dosed to the subject can comprise a single prebiotic or a mixture of prebiotics, and/or a first composition dosed to the subject can comprise a different prebiotic or prebiotics than a second composition dosed to the subject.

As examples, when multiple doses are provided, a first composition comprising a prebiotic can include a first prebiotic, e.g., inulin, and a second composition can include a different prebiotic, e.g., fructooligosaccharide, with or without the first prebiotic. Alternately, a first composition can include a combination of prebiotics, e.g., inulin and fructooligosaccharide and a second composition can include a different combination of prebiotics, e.g., inulin and white navy bean powder. A first composition can include a combination of prebiotics and a second composition can include only one prebiotic.

The amount of prebiotic included in a composition depends on the specific prebiotic, the specific bacterial strain or strains targeted by the prebiotic, and/or the disease state of the subject/patient.

In some aspects, an additional therapeutic agent be incorporated into a pharmaceutical composition is an antidiarrheal agent. Non-limiting examples of antidiarrheal agents suitable for inclusion in a pharmaceutical composition described herein include, but are not limited to, DPP-IV inhibitors, natural opioids, such as tincture of opium, paregoric, and codeine, synthetic opioids, such as diphenoxylate, difenoxin and loperamide, bismuth subsalicylate, lanreotide, vapreotide and octreotide, motiln antagonists, COX2 inhibitors like celecoxib, glutamine, thalidomide and traditional antidiarrheal remedies, such as kaolin, pectin, berberine and muscarinic agents, and a combination thereof.

In some aspects, the additional therapeutic agent incorporated into a pharmaceutical composition can be an analgesic. Analgesics useful in the compositions and methods described herein include, without limitation, morphine, codeine, heroine, methadone and related compounds, thebaine, orpiavine, and their derivatives, buprenorphine, the piperidines, morphinans, benzomorphans, tetrahydroisoquinolines, thiambutanes, benzylamines, tilidine, viminol, nefopam, capsaicin(8-methyl-N-vanillyl-6E-nonenamide), “synthetic” capsaicin(N-vanillylnonamide) and related compounds, and a combination thereof.

In some aspects, the additional therapeutic agent is an anti-bacterial agent, which includes, but is not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); carbapenem antibiotics (ertapenem, doripenem, imipenem/cilastatin, and meropenem); and a combination thereof. In some aspects, the anti-bacterial agent can be any of the penicillin, cephalosporin, monobactam, and carbapenem antibiotics, or a combination thereof.

In one aspect, a method further comprises pretreating a subject with an antibiotic composition prior to administering a therapeutic bacterial mixture. In one aspect, an antibiotic composition administered herein comprises an antibiotic selected from the group consisting of rifabutin, clarithromycin, clofazimine, vancomycin, rifampicin, nitroimidazole, chloramphenicol, and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth sub salicylate, vancomycin, streptomycin, fidaxomicin, amikacin, arbekacin, neomycin, netilmicin, paromomycin, rhodostreptomycin, tobramycin, apramycin, and a combination thereof. In another aspect, a subject is not pretreated with an antibiotic composition prior to administering a bacterial mixture. In another aspect, the pharmaceutical composition is not supplemented with an antibiotic composition. In a further aspect, a method further comprises pretreating a subject with an anti-inflammatory drug prior to administration of a bacterial mixture. In yet another aspect, a subject is not pretreated with an anti-inflammatory drug prior to administering a bacterial or mixture. In another aspect, a bacterial mixture is not supplemented with an anti-inflammatory.

Delivery of an additional therapeutic agent can be targeted to various parts of the GI tract, as described herein.

Use of Pharmaceutical Compositions

Disclosed herein are pharmaceutical compositions that can be administered to a subject to treat or prevent a condition, disorder or disease by increasing an amount of one or more SCFAs in the intestine of the subject. In an aspect, administration of a composition described herein increases a concentration or level of SCFA in the intestine of the subject by administering bacteria (e.g., in a preparation of uncultured fecal bacteria) that have been selected to produce and/or secrete one or more SCFAs in the intestine of the subject following administration of the pharmaceutical composition. In an aspect, administration of a pharmaceutical composition comprising a bacterial mixture described herein treats or prevents a disorder by delivering one or more SCFAs to the intestine of the subject, via the engraftment in the intestine of bacterial strains contained within the administered bacterial mixture, and the subsequent secretion of SCFA by the engrafted bacterial strains. Thus the pharmaceutical compositions described herein are advantageous over compositions that administer a finite amount of an SCFA alone (i.e., in the absence of an SCFA-producing bacterial strain), as administration of the bacterial mixtures and subsequent engraftment of the bacterial strains contained therein results in a continuous, self-sustaining, long-term supply of SCFAs to a subject without the need for regular (e.g., daily) replenishing administrations of the SCFA.

In aspects, a pharmaceutical composition can treat or prevent a disorder that is caused by a reduction in a level of one or more SCFAs in the intestine of the subject. In such cases, administration of a composition can increase the level of the one or more SCFAs in the intestine to directly treat the disorder, resulting in a reduction in a severity of one or more symptoms of the disorder. In aspects, a pharmaceutical composition can treat or prevent a disorder that is not directly caused by a reduction in a level of one or more SCFAs, but has one or more symptoms that are responsive to, or can benefit from, an increase in SCFAs in the intestine of the subject (e.g., the severity of one or more symptoms of the disorder is reduced as a result of an increase in one or more SCFAs in the intestine, even though the disorder itself may remain).

In various aspects, provided herein is a method of modulating a microbiome of a subject in need thereof to provide or restore an ecological balance, comprising administering to the subject a composition described herein. For instance, in various aspects, there is provided methods of diminishing or inhibiting one or more pathogenic bacteria by administering a composition described herein. In various aspects, administration of one or more bacterial isolates described herein augments growth of at least one type of bacteria not detectably present in a patient's GI tract prior to administration and, in various aspects, which is non-pathogenic.

In various aspects, provided herein is a method of restoring or enhancing ecological control over gut pathogens or pathobionts in a subject in need thereof, comprising administering to the subject a composition described herein.

In aspects, the pharmaceutical composition is for administration to a subject having a disorder related to an intestinal dysbiosis. In aspects, the disorder is selected from the group consisting of inflammatory bowel disease (IBD), irritable bowel syndrome (IBS), C. difficile infection (CDI), C. difficile-associated disease (CDAD), an antibiotic-induced adverse effect, and a combination thereof.

In various aspects, a method comprises administering a composition described herein to treat a disease or condition associated with GI dysbiosis in a subject in need thereof. In some aspects, the subject has inflammatory bowel disease (IBD), for example, Crohn's disease, colitis (e.g., ulcerative colitis or microscopic colitis), or pouchitis. IBD is a group of inflammatory conditions of the large intestine and, in some cases, the small intestine. Examples of IBD that can be treated by the compositions, formulations and methods described herein include, but are not limited to, Crohn's disease, ulcerative colitis, microscopic colitis, pouchitis, collagenous colitis, lymphocytic colitis, ischemic colitis, diversion colitis, Behcet's syndrome, infective colitis, and indeterminate colitis. In an aspect, provided herein is a method of treating ulcerative colitis comprising administering a composition described herein to a subject in need thereof. In another aspect, provided herein is a method of treating Crohn's Disease comprising administering a composition described herein to a subject in need thereof. In a further aspect, provided herein is a method of treating pouchitis comprising administering a composition described herein to a subject in need thereof.

In various aspects, a method comprises administering a composition described herein to treat ulcerative colitis (UC) in a subject in need thereof. UC is one form of IBD. It is a chronic disease of the colon, in which the lining of the colon becomes inflamed and develops tiny open sores, or ulcers, that produce pus and mucous. In some aspects, methods described herein can ameliorate, reduce, or eliminate the inflammation and/or ulceration associated with UC. In some aspects, methods described herein can ameliorate, reduce, or eliminate one or more symptoms associated with UC including but not limited to, abdominal discomfort or pain, frequent emptying of the colon, lose and urgent bowel movements, persistent diarrhea, bloody stool, loss of appetite, and weight loss. In some aspects, methods described herein can reduce or prevent the delay in growth and development in children afflicted with UC.

In various aspects, a method comprises administering a composition described herein to treat UC in a subject in need thereof. For example, a successful treatment of the subject can be measured using the indices below, e.g., the present methods cause a subject's activity score threshold to change from severe to moderate, mild, or remission; or cause a patient's score to change from moderate to mild or remission; or cause a patient's score to change from mild to remission (see Table 1).

TABLE 1

| | Parameters | Scoring | Activity score thresholds | | | |
|---|---|---|---|---|---|---|
| Index | assessed | system | Remission | Mild | Moderate | Severe |
| Mayo score | Stool frequency | Cumulative | 0-2 | 3-5 | 6-10 | 11-12 |
| | Rectal bleeding | score | | | | |
| | Physician's global | | | | | |
| | assessment | | | | | |
| | Sigmoidoscopy | | | | | |
| UCDAI | Stool frequency | Cumulative | 0-2 | 3-8 | | 9-12 |
| | Rectal bleeding | score | | | | |
| | Physician's global | | | | | |
| | assessment | | | | | |
| | Sigmoidoscopy | | | | | |
| Rachmilewitz | Bowel movement | Cumulative | 0-4 | 5-10 | 11-17 | >17 |
| score (CAI) | frequency | score | | | | |
| | Blood in stools | | | | | |
| | Physician's global | | | | | |
| | assessment | | | | | |
| | Abdominal | | | | | |
| | pain/cramps | | | | | |
| | Temperature | | | | | |
| | EIMs | | | | | |
| | Laboratory findings | | | | | |
| | (ESR, hemoglobin) | | | | | |
| Powell-Tuck | Well-being | Cumulative | ≤3 | 4-10 | 11-14 | >14 |
| index (St | Abdominal pain | score | | | | |
| Mark's index) | Bowel movement | | | | | |
| | frequency | | | | | |
| | Stool consistency | | | | | |
| | Bleeding | | | | | |
| | Anorexia | | | | | |
| | Nausea/vomiting | | | | | |
| | Abdominal | | | | | |
| | tenderness | | | | | |
| | Eye, joint, mouth, | | | | | |
| | or skin | | | | | |
| | complications | | | | | |
| | Temperature | | | | | |
| | Sigmoidoscopy | | | | | |
| SCCAI | Bowel movement | Cumulative | ≤2 | 3-20 | | |
| (Walmsley) | frequency (day) | score | <2.5 | | | |
| | Bowel movement | | | | | |
| | frequency (night) | | | | | |
| | Urgency of | | | | | |
| | defecation | | | | | |
| | Blood in stool | | | | | |
| | Well-being | | | | | |
| | Extracolonic | | | | | |
| | features | | | | | |
| Lichtiger | Diarrhea frequency | Cumulative | ≤3 | 4-8 | 9-14 | >14 |
| index | Nocturnal diarrhea | score | | | | |
| | Visible blood (% of | | | | | |
| | movements) | | | | | |
| | Fecal incontinence | | | | | |
| | Abdominal | | | | | |
| | pain/cramping | | | | | |
| | Well-being | | | | | |
| | Abdominal | | | | | |
| | tenderness | | | | | |
| | Need for | | | | | |
| | antidiarrheal | | | | | |
| | medications | | | | | |
| Seo index | Bowel movement | Cumulative | <108 | <150 | 150-220 | >220 |
| | frequency | score with | <120 | | | |
| | Blood in stool | components | | | | |
| | ESR | given | | | | |
| | Hemoglobin | different | | | | |
| | Albumin | weightings | | | | |
| | | (+constant to | | | | |
| | | yield a mean | | | | |
| | | value as close | | | | |
| | | as possible to | | | | |
| | | Truelove- | | | | |
| | | Witts criteria) | | | | |

In some aspects, a method comprises administering a pharmaceutical composition described herein to treat irritable bowel syndrome (IBS) in a subject in need thereof. IBS is a common disorder that affects the colon and can cause cramping, abdominal pain, bloating, gas, diarrhea and constipation. IBS is classified based on the predominant symptom of diarrhea (IBS with predominant diarrhea, IBS-D), constipation (IBS with predominant constipation, IBS-C) or mixed symptoms (IBS with alternating constipation and diarrhea, IBS-A). Methods described herein can be effective in treating one or more of IBS-D, IBS-C, and/or IBS-A. In some aspects, methods described herein (e.g., comprising administering a composition described herein) can reduce or eliminate one or more symptoms associated with one or more of IBS-D, IBS-C, and/or IBS-A.

In aspects, a method comprises administering a pharmaceutical composition described herein to treat or prevent a disease/disorder associated with an abnormal enteric microflora (e.g. intestinal dysbiosis) in a subject in need thereof. The disease/disorder can be selected from a gastro-intestinal disorder including irritable bowel syndrome or spastic colon, Functional Bowel Disease (FBD), including constipation predominant FBD, pain predominant FBD, upper abdominal FBD, Nonulcer Dyspepsia (NUD), gastro-esophageal reflux, inflammatory bowel disease including Crohn's disease, ulcerative colitis, indeterminate colitis, collagenous colitis, microscopic colitis, chronic Clostridium difficile infection, pseudomembranous colitis, mucous colitis, antibiotic associated colitis, idiopathic or simple constipation, diverticular disease, AIDS enteropathy, small bowel bacterial overgrowth, coeliac disease, polyposis coil, colonic polyps, chronic idiopathic pseudo obstructive syndrome, and toxic megacolon.

In aspects, a method comprises administering a composition described herein to treat or prevent a disorder associated with a liver disorder in a subject in need thereof. Non-limiting examples of a liver disorder include primary biliary cirrhosis, Primary Sclerosing Cholangitis (PSC), fatty liver, and cryptogenic cirrhosis. In aspects, such diseases/disorders are related to an intestinal dysbiosis of a subject.

In aspects, a method comprises administering a composition described herein to treat or prevent a rheumatic disorder in a subject in need thereof. Non-limiting examples of a rheumatic disorder include rheumatoid arthritis, non-rheumatoid arthritis, non-rheumatoid factor positive arthritis, ankylosing spondylitis, Lyme disease, and Reiter's syndrome. In aspects, such diseases/disorders are related to an intestinal dysbiosis of a subject.

In aspects, a method comprises administering a composition described herein to treat or prevent an immune-mediated disorder in a subject in need thereof. Non-limiting examples of an immune-mediated disorder include glomerulonephritis, hemolytic uraemic syndrome, juvenile diabetes mellitus, mixed cryoglobulinaemia, polyarteritis, familial Mediterranean fever, amyloidosis, scleroderma, systemic lupus erythematosus, and Behcets syndrome. In aspects, such diseases/disorders are related to an intestinal dysbiosis of a subject.

In aspects, a method comprises administering a composition described herein to treat or prevent an autoimmune disorder in a subject in need thereof. Non-limiting examples of an autoimmune disorder include Acute Disseminated Encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, Antiphospholipid Syndrome (APS), autoimmune angioedema, autoimmune aplastic anemia, autoimmune dysautonomia, autoimmune hemolytic anemia, autoimmune hepatitis, autoimmune hyperlipidemia, autoimmune immunodeficiency, Autoimmune Inner Ear Disease (AIED), autoimmune myocarditis, autoimmune oophoritis, autoimmune pancreatitis, autoimmune retinopathy, Autoimmune Thrombocytopenic Purpura (ATP), autoimmune thyroid disease, autoimmune urticarial, axonal & neuronal neuropathies, Balo disease, Behcet's disease, bullous pemphigoid, cardiomyopathy, Castleman disease, celiac disease, Chagas disease, Chronic Inflammatory Demyelinating Polyneuropathy (CIDP), Chronic Recurrent Multifocal Ostomyelitis (CRMO), Churg-Strauss syndrome, cicatricial pemphigoid/benign mucosal pemphigoid, Crohn's disease, Cogan's syndrome, cold agglutinin disease, congenital heart block, Coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibrosing alveolitis, giant cell arteritis (temporal arteritis), giant cell myocarditis, glomerulonephritis, Goodpasture's syndrome, Granulomatosis With Polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, immunoregulatory lipoproteins, inclusion body myositis, interstitial cystitis, juvenile arthritis, juvenile idiopathic arthritis, juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (systemic lupus erythematosus), chronic Lyme disease, Meniere's disease, microscopic polyangiitis, Mixed Connective Tissue Disease (MCTD), Mooren's ulcer, Mucha-Habermann disease, multiple sclerosis, myasthenia gravis, myositis, narcolepsy, neuromyelitis optica (Devi c's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, Paroxysmal Nocturnal Hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, Primary Sclerosing Cholangitis (PSC), psoriasis, psoriatic arthritis, idiopathic pulmonary fibrosis, pyoderma gangrenosum, pure red cell aplasia, Raynaud's phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjögren's syndrome, sperm & testicular autoimmunity, stiff person syndrome, Subacute Bacterial Endocarditis (SBE), Susac's syndrome, sympathetic ophthalmia, Takayasu's arteritis, temporal arteritis/giant cell arteritis, Thrombocytopenic Purpura (TTP), Tolosa-Hunt syndrome, transverse myelitis, type 1 diabetes, asthma, ulcerative colitis, Undifferentiated Connective Tissue Disease (UCTD), uveitis, vasculitis, vesiculobullous dermatosis, vitiligo, and Wegener's granulomatosis. In aspects, such disorders are related to an intestinal dysbiosis of a subject.

In aspects, a method comprises administering a composition described herein to treat or prevent a neurological syndrome in a subject in need thereof. Non-limiting examples of a neurological syndrome include as chronic fatigue syndrome, migraine, multiple sclerosis, amyotrophic lateral sclerosis, myasthenia gravis, Gillain-Barré syndrome, Parkinson's disease, Alzheimer's disease, Chronic Inflammatory Demyelinating Polyneuropathy, and other degenerative disorders. In aspects, such syndromes are related to an intestinal dysbiosis of a subject.

In aspects, a method comprises administering a composition described herein to treat or prevent a psychiatric or mental disorder in a subject in need thereof. Non-limiting examples of a psychiatric or mental disorder include chronic depression, schizophrenia, psychotic disorders, manic depressive illness; regressive disorders including, Asperger's syndrome, Rett syndrome, Attention Deficit Hyperactivity Disorder (ADHD), Attention Deficit Disorder (ADD), the regressive disorder, autism, Sudden Infant Death Syndrome (SIDS), and anorexia nervosa. In aspects, such diseases/disorders are related to an intestinal dysbiosis of a subject.

In aspects, a method comprises administering a composition described herein to treat or prevent a dermatological condition in a subject in need thereof. Non-limiting examples of a dermatological condition include chronic urticaria, acne, eczema, atopic dermatitis, contact dermatitis, dermatitis herpetiformis and vasculitis disorders. In aspects, such diseases/disorders are related to an intestinal dysbiosis of a subject.

In aspects, a method comprises administering a composition described herein to treat or prevent a cardiovascular and/or vascular disorder in a subject in need thereof. In aspects, such diseases/disorders are related to an intestinal dysbiosis of a subject.

In aspects, a method comprises administering a composition described herein to treat or prevent a bloodstream infection (BSI) in a subject in need thereof. Patients at risk for such BSIs include but are not limited to solid organ transplant patients; chronic kidney disease patients, e.g., on hemodialysis; and oncology patients. In aspects, such BSIs are related to an intestinal dysbiosis of a subject.

In aspects, a method comprises administering a composition described herein to treat or prevent a catheter or intravascular-line infection (e.g., central-line infection) in a subject in need thereof. In aspects, such infections are related to an intestinal dysbiosis of a subject.

In aspects, a method comprises administering a composition described herein to treat or prevent a skin or soft tissue infection in a subject in need thereof. In aspects, such infections are related to an intestinal dysbiosis of a subject.

In aspects, a method comprises administering a composition described herein to treat or prevent a surgical-site infection in a subject in need thereof. In aspects, such infections are related to an intestinal dysbiosis of a subject.

In aspects, a method comprises administering a composition described herein to treat or prevent a urinary tract infection (e.g., antibiotic-resistant urinary tract infections and catheter-associated urinary tract infections) in a subject in need thereof. In aspects, such infections are related to an intestinal dysbiosis of a subject.

In aspects, a method comprises administering a composition described herein to treat or prevent a wound infection in a subject in need thereof. In aspects, such infections are related to an intestinal dysbiosis of a subject.

In aspects, a method comprises administering a composition described herein to treat or prevent an infection in a subject in need thereof. Non-limiting examples of an infection include an antibiotic-resistant infection and an antibiotic-sensitive infection. In aspects, such infections are related to an intestinal dysbiosis of a subject.

In aspects, the pharmaceutical compositions and methods described herein can treat or prevent meningitis. In aspects, the meningitis is related to an intestinal dysbiosis of a subject.

In aspects, a method comprises administering a composition described herein to treat or prevent pneumonia, e.g., ventilator-associated pneumonia in a subject in need thereof. In aspects, the pneumonia is related to an intestinal dysbiosis of a subject.

In aspects, the compositions, formulations and methods described herein can be used in patient populations who are in an outpatient setting, hospitalized, and/or in long-term care facilities. Such patient populations are at risk for nosocomial infections. In aspects, such infections are related to an intestinal dysbiosis of a subject.

In aspects, a method comprises administering a composition described herein to treat or prevent Primary Sclerosing Cholangitis (PSC) in a subject in need thereof. For example, one or more bacterial isolates provided in a pharmaceutical composition administered to the subject can replace a dysbiotic gut microbiome with a healthy community, thereby, at least, reducing bile duct inflammation and/or improving liver function.

In aspects, a method comprises administering a composition described herein to treat or prevent a diarrheal disease in a subject in need thereof. Non-limiting examples of a diarrheal disease include acute bloody diarrhea (e.g., dysentery), acute watery diarrhea (e.g., cholera), checkpoint inhibitor associated colitis, diarrhea due to food poisoning, persistent diarrhea, and traveler's diarrhea. In aspects, the diarrhea is related to an intestinal dysbiosis of a subject.

In various aspects, administration of a pharmaceutical composition described herein can reduce, ameliorate, or eliminate one or more symptom(s) associated with a herein-described condition, disease, or disorder. Exemplary symptoms include, but are not limited to, diarrhea, bloody stool, mouth sores, perianal disease, abdominal pain, abdominal cramping, fever, fatigue, weight loss, iron deficiency, anemia, appetite loss, weight loss, anorexia, delayed growth, delayed pubertal development, and inflammation of the skin, eyes, joints, liver, and bile ducts. In aspects, the symptom is related to an intestinal dysbiosis of a subject.

In some aspects, a method comprises administering a composition described herein to treat or prevent an infection by pathogenic bacteria and/or inhibit the growth or decrease the number of pathogenic bacteria in the GI tract of a subject in need thereof. In an aspect, the pathogenic bacteria is enterobacteria such as Salmonella. In various aspects, a method comprises administering a composition described herein to mitigate or prevent the overgrowth of various coliforms in a patient's gut (including coliforms that are virulent and/or antibiotic resistant). Illustrative coliforms include Citrobacter, Enterobacter, Hafnia, Kelbsiella, and Escherichia. In some aspects, the methods and compositions described herein prevent or diminish secondary infections with resistant organisms.

In still other aspects, a method comprises administering a composition described herein to treat or prevent an infectious disease of the intestines in a subject in need thereof. Non-limiting examples of an infectious disease of the intestine include CDI and/or a CDAD, nosocomial infection, secondary emergent infection, amebiasis, intestinal tuberculosis, or parasitic disorder. In some aspects, provided herein are methods for treating or preventing a CDI and/or a CDAD, comprising administering an effective amount of a pharmaceutical composition described herein to a subject or a patient need thereof. In various aspects, the CDI or CDAD comprises one or more of: C. difficile diarrhea (CDD), C. difficile intestinal inflammatory disease, colitis, pseudomembranous colitis, fever, abdominal pain, dehydration and disturbances in electrolytes, megacolon, peritonitis, and perforation and/or rupture of the colon.

In various aspects, a composition described herein is administered to a subject in need thereof to treat or prevent a disease or condition associated with GI dysbiosis in the context of initial onset or relapse/recurrence (e.g. due to continued or restarted antibiotic therapy). For example, in a subject that has previously suffered from a GI dysbiosis, the present pharmaceutical composition or formulation can be administered upon the first symptoms of recurrence in the subject. By way of non-limiting example, symptoms of recurrence include, in a mild case, about 5 to about 10 watery bowel movements per day, no significant fever, and only mild abdominal cramps while blood tests can show a mild rise in the white blood cell count up to about 15,000 (normal levels are up to about 10,000), and, in a severe case, more than about 12 watery stools per day, nausea, vomiting, high fever (e.g. about 102-104° F.), rectal bleeding, severe abdominal pain (e.g. with tenderness), abdominal distention, and a high white blood count (e.g. of about 15,000 to about 40,000).

In some aspects, the methods described herein can be used to treat a subject or patient who is suffering from, or is susceptible to, a disease or condition associated with GI dysbiosis. For example, the subject can be undergoing or have undergone an initial and/or adjunctive therapy that renders the subject susceptible to a disease or condition associated with GI dysbiosis. In some aspects, the subject is undergoing treatment, or has undergone treatment, with an antibiotic. For example, the subject can have taken an antibiotic during the past about 30 days and/or have an immune system that is weak (e.g. from a chronic illness). In another example, the patient can have recently been in the hospital, including in an intensive care unit. Accordingly, in some aspects, a method comprises administering a composition described herein to treat or prevent a nosocomial infection and/or a secondary emergent infection and/or a hospital acquired infection (HAI) in a subject in need thereof.

In various aspects, described herein are methods for treating antibiotic-induced adverse effects in the GI tract, comprising administering an effective amount of a microbial therapeutic (e.g., one or more bacterial isolates) to a subject in need thereof. In another aspect, provided herein are methods for preventing an antibiotic-induced adverse effect in the GI tract, comprising administering an effective amount of a microbial therapeutic to a subject in need thereof.

In another aspect, a pharmaceutical composition or a plurality of pharmaceutical compositions, as disclosed herein, can be used in the manufacture of a medicament, e.g., for treating a herein-described condition, disease, or disorder in a subject in need thereof. In various aspects, the bacterial isolates as described herein protect the intestinal microbiome from antibiotics-induced damage. In some aspects, the methods described herein can treat or prevent an antibiotics-associated adverse effect including but not limited to diarrhea, nausea, vomiting, dysgeusia, colitis, and pseudomembranous colitis disease and/or symptoms. In an aspect, methods described herein can be used to treat or prevent antibiotic-associated diarrhea (AAD).

Provided herein is a method of delivering one or more SCFAs to the intestine of a subject in need thereof, the method comprising administering to the subject a pharmaceutical composition comprising a bacterial mixture comprising one or more SCFA-producing bacterial strains, such that the one or more SCFA-producing bacterial strains engraft in the intestine of the subject and release the SCFA into the subject's intestine.

Provided herein is a method of delivering one or more SCFAs to the intestine of a subject in need thereof, the method comprising administering to the subject a bacterial mixture comprising uncultured fecal bacteria selected to produce the one or more SCFAs, such that SCFA-producing bacterial strains of the uncultured fecal bacteria engraft in the intestine of the subject and release the SCFA into the subject's intestine. In an aspect, a relative abundance of one or more SCFA-producing bacterial strains in the intestine of the subject following administration of the pharmaceutical composition is greater than the relative abundance of the one or more SCFA-producing bacterial strains in the intestine of the subject prior to administration of the composition.

Provided herein is a method of delivering one or more SCFAs to the intestine of a subject in need thereof, the method comprising determining or diagnosing a level of an SCFA produced by intestinal bacteria of the subject (i.e., the level of the SCFA molecules produced and/or secreted by the intestinal bacteria is determined), and administering to the subject a pharmaceutical composition described herein based on the level of the SCFA produced by the intestinal bacteria. In an aspect, the pharmaceutical composition is administered if the level of the SCFA produced by the intestinal bacteria is below a threshold level.

In an aspect, a presence or level of SCFA produced by the intestinal bacteria of the subject is determined directly from stool of the subject (e.g., in a direct SCFA quantification assay using gas chromatography).

In as aspect, the SCFA is butyrate, and a pharmaceutical composition described herein is administered to the subject if a level or presence of butyrate in the stool of the subject is determined to be below a threshold level. In aspects, the threshold level of butyrate in the stool of a subject (i.e., below which a pharmaceutical composition is administered) is 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 nmol per gram of fresh or raw stool.

In an aspect, the SCFA is acetate, and a pharmaceutical composition described herein is administered to the subject if a level or presence of acetate in the stool of the subject is determined to be below a threshold level. In aspects, the threshold level of acetate in the stool of a subject (i.e., below which a pharmaceutical composition is administered) is 70, 65, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 nmol per gram of fresh or raw stool.

In an aspect, the SCFA is caproate, and a pharmaceutical composition described herein is administered to the subject if a level or presence of caproate in the stool of the subject is determined to be below a threshold level. In aspects, the threshold level of caproate in the stool of a subject (i.e., below which a pharmaceutical composition is administered) is 8, 7.9, 7.8, 7.7, 7.6, 7.5, 7.4, 7.3, 7.2, 7.1, 7, 6.9, 6.8, 6.7, 6.6, 6.5, 6.4, 6.3, 6.2, 6.1, 6, 5.9, 5.8, 5.7, 5.6, 5.5, 5.4, 5.3, 5.2, 5.1, 5, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2 or 0.1 nmol per gram of fresh or raw stool.

In an aspect, the SCFA is heptanoate, and a pharmaceutical composition described herein is administered to the subject if a level or presence of heptanoate in the stool of the subject is determined to be below a threshold level. In aspects, the threshold level of heptanoate in the stool of a subject (i.e., below which a pharmaceutical composition is administered) is 11, 10.5, 10, 9.5, 9, 8.9, 8.8, 8.7, 8.6, 8.5, 8.4, 8.3, 8.2, 8.1, 8, 7.9, 7.8, 7.7, 7.6, 7.5, 7.4, 7.3, 7.2, 7.1, 7, 6.9, 6.8, 6.7, 6.6, 6.5, 6.4, 6.3, 6.2, 6.1, 6, 5.9, 5.8, 5.7, 5.6, 5.5, 5.4, 5.3, 5.2, 5.1, 5, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2 or 0.1 nmol per gram of fresh or raw stool.

In an aspect, the SCFA is isobutyrate, and a pharmaceutical composition described herein is administered to the subject if a level or presence of isobutyrate in the stool of the subject is determined to be below a threshold level. In aspects, the threshold level of isobutyrate in the stool of a subject (i.e., below which a pharmaceutical composition is administered) is 5, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2 or 0.1 nmol per gram of fresh or raw stool.

In an aspect, the SCFA is isocaproate, and a pharmaceutical composition described herein is administered to the subject if a level or presence of isocaproate in the stool of the subject is determined to be below a threshold level. In aspects, the threshold level of isocaproate in the stool of a subject (i.e., below which a pharmaceutical composition is administered) is 5, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2 or 0.1 nmol per gram of fresh or raw stool.

In an aspect, the SCFA is isovalerate, and a pharmaceutical composition described herein is administered to the subject if a level or presence of isovalerate in the stool of the subject is determined to be below a threshold level. In aspects, the threshold level of isovalerate in the stool of a subject (i.e., below which a pharmaceutical composition is administered) is 8, 7.9, 7.8, 7.7, 7.6, 7.5, 7.4, 7.3, 7.2, 7.1, 7, 6.9, 6.8, 6.7, 6.6, 6.5, 6.4, 6.3, 6.2, 6.1, 6, 5.9, 5.8, 5.7, 5.6, 5.5, 5.4, 5.3, 5.2, 5.1, 5, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2 or 0.1 nmol per gram of fresh or raw stool.

In as aspect, the SCFA is propionate, and a pharmaceutical composition described herein is administered to the subject if a level or presence of propionate in the stool of the subject is determined to be below a threshold level. In aspects, the threshold level of propionate in the stool of a subject (i.e., below which a pharmaceutical composition is administered) is 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 nmol per gram of fresh or raw stool.

In an aspect, the SCFA is valerate, and a pharmaceutical composition described herein is administered to the subject if a level or presence of valerate in the stool of the subject is determined to be below a threshold level. In aspects, the threshold level of valerate in the stool of a subject (i.e., below which a pharmaceutical composition is administered) is 7, 6.9, 6.8, 6.7, 6.6, 6.5, 6.4, 6.3, 6.2, 6.1, 6, 5.9, 5.8, 5.7, 5.6, 5.5, 5.4, 5.3, 5.2, 5.1, 5, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2 or 0.1 nmol per gram of fresh or raw stool.

In an aspect, a presence or level of SCFA produced by the intestinal bacteria of the subject is determined in a functional assay after extracting or harvesting the intestinal bacteria from stool of the subject (e.g., in an “ex vivo” assay that directly determines the capability of intestinal bacteria to produce one or more SCFAs). For example, fecal bacteria can be extracted from stool of a subject (e.g., by filtering and/or centrifuging), incubated for a period of time (e.g., at least 1 at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 hours, or greater than 24 hours) with one or more substrates (e.g., one or more carbohydrate substrates such as inulin, an oligosaccharide, sunfiber, and/or white navy bean powder), and a presence or level of an SCFA produced by the fecal bacteria by metabolizing the substrate(s) determined using gas chromatography). In an aspect, a pharmaceutical composition described herein can be administered to the subject based on a determination that the SCFA produced by the fecal bacteria (e.g., in a functional assay such as an ex vivo assay) is below a threshold level.

In an aspect, the SCFA is butyrate, and a pharmaceutical composition described herein is administered to the subject if a level of butyrate produced by fecal bacteria of the subject as determined by functional assay is below a threshold level. In aspects, the threshold level of butyrate (i.e., below which a pharmaceutical composition is administered) is 40, 35, 30, 29, 28, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 mM.

In an aspect, the SCFA is acetate, and a pharmaceutical composition described herein is administered to the subject if a level of acetate produced by fecal bacteria of the subject as determined by a functional assay is below a threshold level. In aspects, the threshold level of acetate (i.e., below which a pharmaceutical composition is administered) is 60, 55, 50, 45, 40, 35, 30, 29, 28, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 mM.

In an aspect, the SCFA is propionate, and a pharmaceutical composition described herein is administered to the subject if a level of propionate produced by fecal bacteria of the subject as determined by a functional assay is below a threshold level. In aspects, the threshold level of propionate (i.e., below which a pharmaceutical composition is administered) is 25, 24, 23, 22, 21, 20, 19, 18, 17, 16, 15, 14, 13, 12, 11, 10, 9, 8, 7, 6, 5, 4, 3, 2, or 1 mM.

In an aspect, a subject administered a pharmaceutical composition (e.g., comprising a bacterial mixture comprising a preparation or uncultured fecal bacteria) on the basis of a determination that intestinal bacteria of the subject produce one or more SCFAs below a threshold level can be monitored and/or tested for an increase in SCFA levels in the subject's intestine following the administration. For example, following administration, a level or presence of one or more SCFAs can be quantified directly from a stool of the subject, or a level or presence of one or more SCFAs produced by the fecal bacteria in a functional assay (e.g., an in vivo assay) can be determined. In various aspects, a determination of a level or presence of one or more SCFAs in the intestine of a subject is performed at least 6 hours, at least 12 hours, at least 18 hours, at least 24 hours, at least 1 day, at least 2 days, at least 3 days, at least 4 days, at least 5 days, at least 6 days, at least 7 days, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, or at least 16 weeks following administration of a pharmaceutical composition.

In an aspect a method comprises determining a level of one or more SCFAs produced by intestinal bacteria of a subject, administering a first dose of a pharmaceutical composition described herein based on a determination that the one or more SCFAs produced by the intestinal bacteria are below a threshold level, determining the level of the one or more SCFAs produced by intestinal bacteria of the subject from a sample (e.g., stool) collected after the pharmaceutical composition is administered, and administering a second dose of the pharmaceutical composition based on a determination that the one or more SCFAs produced by the intestinal bacteria are below a threshold level.

In one aspect of the present disclosure, methods provide for treating a subject in need thereof comprising: (1) administering to the subject a first pharmaceutically active dose of a pharmaceutical composition comprising a preparation of uncultured fecal bacteria of a single donor; (2) testing of the subject to determine efficacy, if an additional dose is necessary, or if the dose should be adjusted; (3) administration of a second pharmaceutical composition comprising a preparation of uncultured fecal bacteria blended from multiple donors; (4) optionally testing of the subject to determine efficacy, if an additional dose is necessary, or if the dose should be adjusted; and (5) optionally administration of a third pharmaceutical composition comprising a preparation of uncultured fecal bacteria blended from multiple donors, where the multiple donors (a) comprise all donors from the second pharmaceutical composition and additional donors, (b) comprise donors of fecal bacteria not included in the second pharmaceutical composition, (c) comprise some but not all of the donors of fecal bacteria included in the second pharmaceutical composition, or comprise donors of fecal bacteria not included in the second pharmaceutical composition. In another aspect, the first, second, and third pharmaceutical compositions are administered in a different order (i.e., first, third, second; third, second, first; third, first, second; second, first, third, etc.).

Methods for measuring change and/or improvement in GI tract function can include, but are not limited to: endoscopy for direct examination of epithelium and mucosa; histological evaluation and/or tissue procurement for direct evaluation of structural changes and/or immune biomarkers; urine tests for assessment of permeability with non-absorbable sugars and LPS levels; stool tests for assessment of inflammation and/or microbiota changes (for example by PCR); and/or blood tests for assessment of specific markers, including CD4+ cell counts, Th17 cell counts, and/or LPS levels.

In aspects, the present disclosure provides a method for treating a disorder (e.g., C. difficile infection, autism spectrum disorder (ASD), ulcerative colitis, Crohn's disease, or another indication listed herein) in a subject in need thereof, where the method comprises administering to the subject a pharmaceutically active dose of a pharmaceutical composition described herein. In aspects, the present disclosure provides a method for treating a disorder (e.g., C. difficile infection, ASD, ulcerative colitis, or Crohn's disease) in a subject in need thereof, where the method comprises administering daily to the subject a pharmaceutically active dose of a pharmaceutical composition described herein. In aspects, a pharmaceutical composition is administered to a patient in need thereof at least once daily for at least two consecutive days. In aspects, a pharmaceutical composition is administered at least once daily for at least 3, 4, 5, 6, 7, 8, 9, 10, 11, 12, 13, 14, or 15 consecutive days. In aspects, a pharmaceutical composition is administered at least once daily for at least 1, 2, 3, 4, 5, 6, 7, 8, 9, 10, 11, or 12 consecutive weeks. In aspects, a pharmaceutical composition is administered at least twice, three times, four times, or five times per week for at least 1, 2, 3, 4, 5, 6, 7, 8, 9, 10, 11, or 12 consecutive weeks. In aspects, a pharmaceutical composition is administered at least once daily for at most 4, 5, 6, 7, 8, 9, 10, 11, 12, 13, 14, 15, 16, 17, 18, 19, or 20 consecutive days or weeks. In aspects, a pharmaceutical composition is administered at least once daily for at most 1, 2, 3, 4, 5, 6, 7, 8, 9, 10, 11, or 12 consecutive weeks or months. In aspects, a pharmaceutical composition is administered at least once for at least 1, 2, 3, 4, 5, 6, 7, 8, 9, 10, 11, or 12 consecutive months or years, chronically for a subject's entire life span, or an indefinite period of time.

In aspects, a pharmaceutical composition is administered to a patient in need thereof at least twice daily for at least two consecutive days. In aspects, a pharmaceutical composition is administered at least twice daily for at least 3, 4, 5, 6, 7, 8, 9, 10, 11, 12, 13, 14, or 15 consecutive days. In aspects, a pharmaceutical composition is administered at least twice daily for at least 1, 2, 3, 4, 5, 6, 7, 8, 9, 10, 11, or 12 consecutive weeks. In aspects, a pharmaceutical composition is administered at least twice daily for at most 4, 5, 6, 7, 8, 9, 10, 11, 12, 13, 14, 15, 16, 17, 18, 19, or 20 consecutive days or week. In aspects, a pharmaceutical composition is administered at least twice daily for at most 1, 2, 3, 4, 5, 6, 7, 8, 9, 10, 11, or 12 consecutive weeks or months. In aspects, a pharmaceutical composition is administered at least twice for at least 1, 2, 3, 4, 5, 6, 7, 8, 9, 10, 11, or 12 consecutive months or years, chronically for a subject's entire life span, or an indefinite period of time.

In aspects, a pharmaceutical composition is administered to a patient in need thereof at least three times daily for at least two consecutive days. In aspects, a pharmaceutical composition is administered at least three times daily for at least 3, 4, 5, 6, 7, 8, 9, 10, 11, 12, 13, 14, or 15 consecutive days. In aspects, a pharmaceutical composition is administered at least three times daily for at least 1, 2, 3, 4, 5, 6, 7, 8, 9, 10, 11, or 12 consecutive weeks. In aspects, a pharmaceutical composition is administered at least three times daily for at most 4, 5, 6, 7, 8, 9, 10, 11, 12, 13, 14, 15, 16, 17, 18, 19, or 20 consecutive days or weeks. In aspects, a pharmaceutical composition is administered at least three times daily for at most 1, 2, 3, 4, 5, 6, 7, 8, 9, 10, 11, or 12 consecutive weeks or months. In aspects, a pharmaceutical composition is administered at least three times for at least 1, 2, 3, 4, 5, 6, 7, 8, 9, 10, 11, or 12 consecutive months or years, chronically for a subject's entire life span, or an indefinite period of time.

In aspects, the present disclosure provides a method for treating a disorder (e.g., C. difficile infection, ASD, ulcerative colitis, or Crohn's disease) in a subject in need thereof, where the method comprises administering orally to the subject a pharmaceutically active dose of a pharmaceutical composition comprising one or more live, non-pathogenic, bacterial isolates described herein, where the dose is administered at a dosing schedule of at least once or twice daily for at least three consecutive days or weeks. In aspects, a dose is administered at least once, twice, or three times daily for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In one aspect, the present disclosure provides a method for treating ASD in a subject in need thereof by administering a pharmaceutical composition described herein, where the method comprises a single dosing schedule. In one aspect, the dosing schedule comprises a treatment period of at least 1, 2, 3, 4, 5, 6, 7, 8, 9, 10, 11, 12, 13, 14, 15, or 16 consecutive weeks. In an aspect, a dosing schedule comprises administering a dose every day, every other day, every two days, or every 3, 4, 5, 6, 7, 8 days.

In one aspect, the present disclosure provides a method for treating a disorder in a subject in need thereof by administering a pharmaceutical composition described herein, where the method comprises a first dosing schedule followed by a second dosing schedule. In one aspect, a first dosing schedule comprises a treatment or induction dose. In one aspect, a first dosing schedule comprises a continuous dosing schedule. In another aspect, a first dosing schedule comprises a dosing schedule of two consecutive days. In another aspect, a first dosing schedule comprises a dosing schedule of two consecutive days of an equivalent dose. In another aspect, a first dosing schedule comprises a dose on a single day. In another aspect, a first dosing schedule comprises a dosing schedule of three consecutive days. In another aspect, a first dosing schedule comprises a dosing schedule of four consecutive days. In another aspect, a first dosing schedule comprises a dosing schedule of five consecutive days. In another aspect, a first dosing schedule comprises a dosing schedule of six consecutive days. In another aspect, a first dosing schedule comprises a dosing schedule of seven consecutive days. In another aspect, a first dosing schedule comprises a dosing schedule of at least 3, 4, 5, 6, 7, 8, 9, 10, 11, or 12 consecutive days. In another aspect, a second dosing schedule comprises a maintenance dose lower than or equal to a pharmaceutically active dose of a first dosing schedule. In another aspect, a second dosing schedule lasts for at least about 2, 4, 5, 6, 7, 8, 9, 10, 11, 12, 18, 24, 36, 48, 72, or 96 weeks. In another aspect, a second dosing schedule comprises a dosing schedule of at least 2, 4, 5, 6, 7, 8, 9, 10, 11, 12, 18, 24, 36, 48, 72, or 96 consecutive weeks. In another aspect, a second dosing schedule comprises a dosing schedule of at least 2, 4, 5, 6, 7, 8, 9, 10, 11, 12, 18, 24, 36, 48, 72, or 96 consecutive weeks. In another aspect, a second dosing schedule comprises a dosing schedule of at least 12, 14, 21, 28, 35, 42, 49, 56, 63, 70, or 77 consecutive days. In one aspect, a second dosing schedule lasts permanently, for a treated subject's entire life span, or an indefinite period of time. In one aspect, a second dosing schedule is a continuous dosing schedule. In another aspect, a second dosing schedule is an intermittent dosing schedule. In a further aspect, a second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In another aspect, a second dosing schedule comprises administering a second dose (e.g., a maintenance dose) every other day, every two days, or every 3, 4, 5, 6, 7, 8 days. In another aspect, a maintenance dose is administered for an extended period of time with or without titration (or otherwise changing the dosage or dosing schedule). In one aspect, there is no interval between a first and a second dosing schedule. In another aspect, the interval between a first and a second dosing schedule is at least 1, 2, 3, 4, 5, 6, or 7 days. In one aspect, the interval between a first and a second dosing schedule is at least about 1, 2, 3, 4, 5, 6, 7, 8, 9, 10, 11, 12, 13, 14, 15 or 16 weeks. In another aspect, a second dosing schedule (e.g., a maintenance dose) comprises a dosage about 2, 3, 4, 5, 10, 50, 100, 200, 400, or 500 folds lower than the dosage used in a first dosing schedule (e.g., an initial treatment dose). In another aspect, a second dosing schedule (e.g., a maintenance dosing schedule) has an equal or lower dosing frequency than a first dosing schedule (e.g., an initial treatment dosing schedule). In another aspect, a second dosing schedule (e.g., a maintenance dosing schedule) has a higher dosing interval than a first dosing schedule (e.g., an initial treatment dosing schedule).

In aspects, the present disclosure provides a method for treating a disorder (e.g., C. difficile infection, ASD, ulcerative colitis, or Crohn's disease) in a subject in need thereof, where the method comprises a combination treatment or therapy. For example, the method can comprise a double combination therapy, a triple combination therapy, or a quadruple combination therapy.

In another aspect, disclosed herein are a plurality of pharmaceutical compositions, e.g., two or more pharmaceutical compositions, as disclosed herein, for use in the prevention or treatment of a condition, disease, or disorder in a subject in need thereof. In aspects, a first composition comprises one or more bacterial isolates described herein. In aspects, a second composition comprises a preparation of uncultured fecal bacteria (e.g., a substantially complete fecal microbiota purified from a stool sample). A subject can be treated with the first and second compositions in any order to treat or prevent a disorder. For example, in one aspect a subject is treated with a composition comprising a preparation of uncultured fecal bacteria, followed by a composition comprising one or more bacterial isolates. In another aspect, a subject is treated with a composition comprising one or more bacterial isolates followed by a composition comprising a preparation of uncultured fecal bacteria. In still other aspects, a subject can be treated with a composition comprising one or more bacterial isolates and a composition comprising a preparation of uncultured fecal bacteria simultaneously (for example, with a composition comprising both the bacterial isolate(s) and the uncultured fecal bacteria, or with multiple compositions each comprising one of the bacterial isolate(s) or a preparation of uncultured fecal bacteria).

In an aspect, a method for treating or preventing a condition, disease or disorder of a subject comprises administration to the subject of: (i) a pharmaceutical composition comprising one or more bacterial isolates; and (ii) a preparation of uncultured fecal bacteria. For example, the one or more bacterial isolates can be administered before or after the uncultured fecal bacteria, or at the same time (e.g., in different compositions or together in the same composition). In another aspect, a method for treating a disorder of a subject comprises administration to the subject of: (i) a pharmaceutical composition comprising a bacterial mixture (e.g., comprising a preparation of uncultured fecal bacteria); and (ii) one or more antibiotics. Typically, the antibiotic is administered to the subject prior to administration of the bacterial mixture, in order to purge the subject's intestine of harmful and/or pathogenic bacteria prior to replenishment of the gut with bacteria from the bacterial mixture. In an aspect, a method for treating a disorder of a subject comprises administration to the subject of: (i) a pharmaceutical composition comprising a bacterial mixture (e.g., comprising a preparation of uncultured fecal bacteria); and (ii) a prebiotic. For example, the bacterial mixture can be administered before or after the prebiotic, or at the same time (e.g., in different compositions or together in the same composition). For each of the above examples, it is further understood that any given component in a method of treatment can be administered multiple times. For example, a preparation of uncultured fecal bacteria can be administered to the subject, followed by one or more bacterial isolates, followed by a second administration of a preparation of uncultured fecal bacteria.

In an aspect, a method for treating or preventing a condition, disease or disorder of a subject comprises administration to the subject of: (i) a pharmaceutical composition comprising one or more bacterial isolates; (ii) a preparation of uncultured fecal bacteria; and (iii) one or more antibiotics. The different components of (i)-(iii) can be administered to the subject in any order. For example, a subject can be administered one or more antibiotics, followed by a preparation of uncultured fecal bacteria, followed by one or more bacterial isolates. In another example, the subject can be administered one or more antibiotics, followed by one or more bacterial isolates, followed by a preparation of uncultured fecal bacteria. For each of the above examples, it is further understood that any given component in a method of treatment can be administered multiple times. For example, an antibiotic can be administered to the subject, followed by a preparation of uncultured fecal bacteria, followed by one or more bacterial isolates, followed by a second administration of a preparation of uncultured fecal bacteria.

In an aspect, a method for treating or preventing a condition, disease or disorder of a subject comprises administration to the subject of: (i) a pharmaceutical composition comprising one or more bacterial isolates; (ii) a preparation of uncultured fecal bacteria; and (iii) one or more prebiotics. The different components of (i)-(iii) can be administered to the subject in any order. For example, a subject can be administered one or more prebiotics, followed by a preparation of uncultured fecal bacteria, followed by one or more bacterial isolates. In another example, the subject can be administered one or more prebiotics, followed by one or more bacterial isolates, followed by a preparation of uncultured fecal bacteria. In another example, the subject can be administered one or more prebiotics following administration of one or both of the one or more bacterial isolates and/or a preparation of uncultured fecal bacteria. For each of the above examples, it is further understood that any given component in a method of treatment can be administered multiple times. For example, a preparation of uncultured fecal bacteria can be administered to a subject, followed by a one or more bacterial isolates, followed by a prebiotic, followed by a second administration of a preparation of uncultured fecal bacteria.

In an aspect, a method for treating or preventing a condition, disease or disorder of a subject comprises administration to the subject of: (i) a pharmaceutical composition comprising one or more bacterial isolates; (ii) a preparation of uncultured fecal bacteria; (iii) one or more prebiotics; and (iv) one or more antibiotics. The different components of (i)-(iv) can be administered to the subject in any order. For example, a subject can be administered one or more antibiotics, followed by one or more prebiotics, followed by a preparation of uncultured fecal bacteria, followed by one or more bacterial isolates. In another example, the subject can be administered one or more antibiotics, followed by one or more prebiotics, followed by one or more bacterial isolates, followed by a preparation of uncultured fecal bacteria. In another example, the prebiotic can be administered after one or both of the one or more bacterial isolates and/or the preparation of uncultured fecal bacteria. For each of the above examples, it is further understood that any given component in a method of treatment can be administered multiple times. For example, an antibiotic can be administered to a subject, followed by a preparation of uncultured fecal bacteria, followed by one or more bacterial isolates, followed by a prebiotic, followed by a second administration of a preparation of uncultured fecal bacteria.

In each of the above combination treatments, the duration of time between different treatments (e.g., between administration of a preparation of uncultured fecal bacteria and one or more bacterial isolates) can be at least 1 hour, at least 2 hours, at least 6 hours, at least 12 hours, at least 1 day, at least 2 days, at least 3 days, at least 4 days, at least 5 days, at least 6 days, at least 1 week, at least 2 weeks, at least 3 weeks, at least 4 weeks, at least 5 weeks, at least 6 weeks, at least 7 weeks, at least 8 weeks, or greater than 8 weeks.

In one aspect, a subject being treated by administering a composition described herein is a subject already with a disorder (e.g., IBD, IBS, or ASD). In another aspect, a pharmaceutical composition is administered to a clinically asymptomatic human subject who is genetically predisposed or prone to a disorder (e.g., IBD, IBS, or ASD) is also useful in preventing the onset of clinical symptoms. A human subject genetically predisposed or prone to a disorder can be a human subject having a close family member or relative exhibiting or having suffered the disorder. In another aspect, a subject being treated by administering a composition described herein is a subject in which a disorder is to be prevented. In another aspect, a subject being treated by administering a composition described herein is predisposed or susceptible to a disorder. In another aspect, a subject being treated by administering a composition described herein is a subject diagnosed as having a disorder. In one aspect, a subject being treated by administering a composition described herein is a patient in need thereof.

In one aspect, a subject being treated by administering a composition described herein is a human patient. In one aspect, a patient is a male patient. In one aspect, a patient is a female patient. In one aspect, a patient is a premature newborn. In one aspect, a patient is a term newborn. In one aspect, a patient is a neonate. In one aspect, a patient is an infant. In one aspect, a patient is a toddler. In one aspect, a patient is a young child. In one aspect, a patient is a child. In one aspect, a patient is an adolescent. In one aspect, a patient is a pediatric patient. In one aspect, a patient is a geriatric patient. In one aspect, a human patient is a child patient below about 18, 15, 12, 10, 8, 6, 4, 3, 2, or 1 year old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old patient (>85 years).

In one aspect, the present disclosure provides a method for treating a subject in need thereof, where the method comprises administering to the subject multiple pharmaceutically active doses of a pharmaceutical composition comprising a bacterial mixture, such that different doses comprise a preparation of uncultured fecal bacteria derived from different carefully screened, healthy donors. In an aspect, a subject is administered a pharmaceutical composition over a dosing period wherein a first dose comprises at least one pharmaceutical composition comprising a preparation of uncultured fecal bacteria of a single donor, and a second dose of a pharmaceutical composition comprises a preparation of uncultured fecal bacteria of a single donor different from the donor of the first dose. In another aspect, a first dose comprises a pharmaceutical composition comprising a preparation of uncultured fecal bacteria of a single donor and a second dose comprises a preparation of uncultured fecal bacteria of a donor pool. The first and second dose do not indicate the order of administration to a subject, but rather that the preparation of uncultured fecal bacteria from separate donors may be used in a non-blended form. In yet another aspect, the preparation of uncultured fecal bacteria from multiple carefully screened, healthy donors is provided in a blended form.

In an aspect, a pharmaceutical composition used herein comprises a bacterial mixture comprising a preparation of uncultured fecal bacteria derived from a donor with preselected desirable characteristics or receiving certain pre-treatment(s). In an aspect, a donor has no current or previous diagnosis or has no symptom of a disorder (e.g., IBD, IBS or ASD) to be treated with a preparation of uncultured fecal bacteria derived from the donor. In another aspect, a donor has no family member or direct relative diagnosed with a disorder or exhibiting a symptom of a disorder to be treated with a preparation of uncultured fecal bacteria derived from the donor. In another aspect, a donor has no siblings, parents, or children diagnosed with or exhibiting a symptom of a disorder to be treated with a preparation of uncultured fecal bacteria derived from the donor. In an aspect, a donor has not previously received any fecal microbiota transplantation. In an aspect, a fecal donor previously donated a stool for treating a GI disorder, e.g., a C. difficile infection or Inflammatory Bowel Disease (IBD).

In an aspect, the present disclosure provides for methods for treating a subject in need thereof by administering to the subject a pharmaceutically active dose of a pharmaceutical composition comprising a preparation of uncultured fecal bacteria of a single donor. In another aspect, the administering is followed by testing to determine the efficacy of the pharmaceutically active dose of the pharmaceutical composition. In another aspect, the testing of the subject provides results to determine if the active dose of the pharmaceutical composition should be adjusted. In another aspect, the testing is followed by administration of a pharmaceutical composition comprising a preparation of uncultured fecal bacteria blended from multiple donors.

In another aspect, the present disclosure provides for methods for treating a subject in need thereof with capsules containing a pharmaceutical composition comprising a preparation of uncultured fecal bacteria from a single donor. In another aspect, a capsule comprises a pharmaceutical composition comprising a preparation of uncultured fecal bacteria from multiple donors. In one aspect, a subject is administered two or more pills comprising a preparation of uncultured fecal bacteria from a single but different donor.

In one aspect, the present disclosure provides for methods for treating a subject in need thereof comprising administering a pharmaceutical composition comprising a preparation of uncultured fecal bacteria of a single donor similar to or different from a prior administration in a treatment period. In another aspect, a treatment period includes administration of a first dose comprising a pharmaceutical composition comprising a preparation of uncultured fecal bacteria of a single donor and administration of a second dose comprising a pharmaceutical composition comprising a preparation of uncultured fecal bacteria of multiple donors.

In an aspect, the present disclosure provides for methods of treating an irritable bowel disease (IBD) in a patient in need thereof comprising administering a preparation of fecal bacteria replete with bile acid transforming strains, wherein the patient in need thereof has a deficient bile acid metabolism, e.g. deficient conversion of primary to secondary bile acids. In another aspect, the present disclosure provides for administering a preparation of fecal bacteria with a relative abundance of secondary bile acids, wherein the patient in need thereof has a deficient bile acid metabolism, e.g., deficient conversion of primary to secondary bile acids. In another aspect, the relative abundance of secondary bile acid in a preparation of fecal bacteria is at or above a threshold level. In an aspect of the present disclosure an irritable bowel disease (IBD) patient is a Crohn's disease patient, a ulcerative colitis patient, a pouchitis patient, or a combination thereof.

In another aspect, the present disclosure provides for methods of treating an irritable bowel disease (IBD) in a patient in need thereof having a deficient bile acid metabolism comprising administering to the patient a preparation of fecal bacteria derived from a donor having a quantity of secondary bile acids above a threshold and bile acid transforming strains.

In an aspect, the present disclosure provides for methods of treating Crohn's disease in a patient with deficient bile acid metabolism. In an aspect, the present disclosure provides for treating the patient with donor materials replete with a quantity of secondary bile acids. In another aspect, the present disclosure provides for treating the patient with donor materials replete with a quantity of bile acid transforming strains. In a further aspect, the present disclosure provides for treating the patient with donor materials replete with a quantity of secondary bile acids and a quantity of bile acid transforming strains. In another aspect, the present disclosure provides for increasing bile acid transforming strains in the patient by administering to the patient a preparation of fecal bacteria derived from a donor having a quantity of bile acid transforming strains at or above a threshold level, wherein the patient's bile acid metabolism is restored.

In an aspect, the present disclosure provides for methods of treating ulcerative colitis in a patient with deficient bile acid metabolism. In an aspect, the present disclosure provides for treating the patient with donor materials replete with a quantity of secondary bile acids. In another aspect, the present disclosure provides for treating the patient with donor materials replete with a quantity of bile acid transforming strains. In a further aspect, the present disclosure provides for treating the patient with donor materials replete with a quantity of secondary bile acids and a quantity of bile acid transforming strains. In another aspect, the present disclosure provides for increasing bile acid transforming strains in the patient by administering to the patient a preparation of fecal bacteria derived from a donor having a quantity of bile acid transforming strains at or above a threshold level, wherein the patient's bile acid metabolism is restored.

In another aspect, the present disclosure provides for methods of treating an ulcerative colitis patient with deficient bile acid metabolism by increasing bile acid transforming strains in the patient comprising administering to the patient a preparation of fecal bacteria derived from a donor having a quantity of bile acid transforming strains at or above a threshold level, wherein the patient's bile acid metabolism is restored. In another aspect, the present disclosure provides for methods of treating a Crohn's disease patient with a deficient bile acid metabolism by increasing bile acid transforming strains in the patient comprising administering to the patient a preparation of fecal bacteria derived from a donor having a quantity of bile acid transforming strains at or above a threshold level, wherein the patient's bile acid metabolism is restored. In another aspect, the present disclosure provides for methods of treating an irritable bowel disease patient with deficient bile acid metabolism by increasing bile acid transforming strains in the patient comprising administering to the patient a preparation of fecal bacteria derived from a donor having a quantity of bile acid transforming strains at or above a threshold level, wherein the patient's bile acid metabolism is restored.

In another aspect, the present disclosure provides methods for delivering a bile acid transforming bacteria to an intestine of a subject in need thereof, the method comprising administering to the subject a pharmaceutical composition comprising a preparation of uncultured fecal bacteria derived from a stool of a healthy human donor, wherein: the uncultured fecal bacteria are selected to transform primary bile acid to secondary bile acid at or above a threshold level, fecal bacteria in a stool of the healthy human donor produce the secondary bile acid at or above a threshold level, or the healthy human donor produces a stool comprising the secondary bile acid at or above a threshold level.

In another aspect, the present disclosure also provides methods determining a level of at least one secondary bile acid produced by fecal bacteria of a subject comprising at least one bile acid transforming bacterial strain; and administering to the subject a pharmaceutical composition based on determining the level of the at least one secondary bile acid to be below a threshold level, wherein the pharmaceutical composition comprises a preparation of uncultured fecal bacteria of stool of a healthy human donor.

In another aspect, the present disclosure also provides method comprising: determining a level of a secondary bile acid transformed by fecal bacterial cells to be at or above a threshold level, wherein the fecal bacterial cells are from a stool of a healthy human donor; and extracting fecal bacteria from a stool of the donor based on determining the level of the secondary bile acid to be at or above the threshold level to produce a preparation of uncultured fecal bacteria.

In a further aspect, the present disclosure provides for methods comprising: determining that a level of secondary bile acids transformed by fecal bacterial cells is at or above a threshold level, wherein the fecal bacterial cells are from a stool of a healthy human donor; selecting fecal bacteria of the donor based on determining that the level of the secondary bile acid is at or above the threshold level, wherein the selected fecal bacteria are uncultured; and mixing the selected fecal bacteria with a cryoprotectant to produce a preparation of uncultured fecal bacteria.

In yet another aspect, the present disclosure provides for methods comprising: determining that a relative abundance of one or more bile acid transforming bacterial strains is at or above a threshold level in stool of a healthy human donor; selecting fecal bacteria of the donor based on determining that the relative abundance of the one or more bile acid transforming bacterial strains is at or above the threshold level, wherein the selected fecal bacteria are uncultured; and mixing the selected fecal bacteria with a cryoprotectant to produce a preparation of uncultured fecal bacteria.

In an even further aspect, the present disclosure provides for a pharmaceutical composition comprising a preparation of fecal bacteria from stool of a healthy human donor, wherein the fecal bacteria transform an amount of primary bile acid to secondary bile acid, wherein the secondary bile acid is at or above a threshold level of secondary bile acid when incubated with a substrate in a functional assay, wherein the preparation of fecal bacteria comprises uncultured bacteria, and wherein the threshold level of the secondary bile acid is at least 100 μM.

In an aspect, the present disclosure provides methods comprising extracting fecal bacteria from a stool of a healthy human donor to produce a preparation of uncultured fecal bacteria, wherein the healthy human donor is pre-selected for secondary bile acid at or above a threshold level in one or more, two or more, three or more, four or more, five or more, six or more, seven or more, or ten or more stool samples from the donor.

In an aspect, a preparation of uncultured fecal bacteria and one or more bacterial isolates are administered to a subject according to a method described herein in the same pharmaceutical composition. In an aspect, a preparation of uncultured fecal bacteria and one or more bacterial isolates are administered to a subject according to a method described herein in different pharmaceutical compositions. In an aspect, multiple bacterial isolates are administered to a subject according to a method described herein in the same pharmaceutical composition. In an aspect, multiple bacterial isolates are administered to a subject according to a method described herein in different pharmaceutical compositions. For example, a method can comprise administering to a subject in need thereof an effective amount of a plurality of pharmaceutical compositions, e.g., two or more pharmaceutical compositions, three or more pharmaceutical compositions, four or more pharmaceutical compositions, or five or more pharmaceutical composition, as disclosed herein. The plurality of pharmaceutical compositions can be provided simultaneously or sequentially. Thus, if a subject is to be treated with, for example, a preparation of uncultured fecal bacteria and two bacterial isolates, a first composition can comprise two of the bacterial isolates and the second composition can comprise the preparation of uncultured fecal bacteria. In a different example, if a subject is to be treated with a preparation of uncultured fecal bacteria and two bacterial isolates, a first composition can comprise the preparation of uncultured fecal bacteria in combination with (or “spiked” with) a first bacterial isolate, and a second composition can comprise the second bacterial isolate. In a different example, if a subject is to be treated with a preparation of uncultured fecal bacteria and three bacterial isolates, a first composition can comprise the first bacterial isolate, a second composition can comprise the second bacterial isolate, a third composition can comprise the third bacterial isolate, and a fourth composition can comprise the preparation of uncultured fecal bacteria.

In one aspect, a method comprises administering a pharmaceutical composition orally, by enema, or via rectal suppository. In one aspect, a pharmaceutical composition is formulated as a geltab, pill, microcapsule, capsule, or tablet. In one aspect, a pharmaceutical composition is formulated as an enteric coated capsule or microcapsule, acid-resistant capsule or microcapsule, or formulated as part of or administered together with a food, a food additive, a dairy-based product, a soy-based product or a derivative thereof, a jelly, or a yogurt. In another aspect, a pharmaceutical composition is formulated as an acid-resistant enteric coated capsule. A pharmaceutical composition can be provided as a powder for sale in combination with a food or drink. A food or drink can be a dairy-based product or a soy-based product. In another aspect, a food or food supplement contains enteric-coated and/or acid-resistant microcapsules containing a pharmaceutical composition.

Further provided herein are kits comprising any herein-disclosed pharmaceutical composition and instructions for use. For example, a kit can include one or more unit dosage forms comprising one or more bacterial mixtures. Such a kit could include for example one or more pharmaceutical compositions comprising a bacterial mixture (e.g., comprising a preparation of fecal bacteria), and optionally a delivery device to administer the composition to the subject, and instructions for administering the dosage to a subject via an appropriate delivery route. In some cases, the dosage form comprises any suitable form of live bacteria (fresh, frozen, lyophilized, etc.) and is formulated for administration to a human subject orally, by nasogastric tube, by colonoscopy, or anally. As described herein, dosage forms suitable for kits provided herein include, without limitation, liquid solutions, capsules, tablets, powders, granules, and lyophilized forms.

The instructions of a kit can describe, for example, dosing information of the one or more pharmaceutical compositions in the kit. As examples, the frequency of administration and dose of a composition, e.g., the number of capsules of a pharmaceutical composition to be administered at a given time, and the number of times of administration per day/week). In an aspect in which the kit comprises more than one composition (e.g., multiple bacterial mixtures or an additional pharmaceutical agent lacking a bacterial mixture), the instructions can describe the dosing of each composition. For example, one composition can be administered before another composition, e.g., sequential administration of the two pharmaceutical compositions separated by minutes, hours, days, weeks, months, or longer. Alternately, two compositions can be administered simultaneously.

Manufacture of Compositions

In a further aspect, provided herein is use of a bacterial mixture described herein for manufacture of a medicament for treating a disorder described herein or for reducing the severity of one or more symptoms of a disorder described herein.

Provided herein are pharmaceutical compositions comprising fecal bacteria (e.g., uncultured fecal bacteria) selected to produce one or more SCFAs in an intestine of a subject administered the composition. In aspects, fecal bacteria of stool of a donor are selected on the basis of a determination that the fecal bacteria are capable of producing one or more SCFAs at or above a threshold level (e.g., determined by quantifying the SCFA from stool of the donor or in an ex vivo assay, or by evaluating the relative abundance of one or more SCFA-producing bacterial strains in stool of the donor). For example, in the case of an ex vivo assay, where fecal bacteria extracted from stool of a donor produce one or more SCFAs at or above a threshold level, then fecal bacteria from stool of the donor (either the same stool used to extract bacteria for the assay or a different stool) can be selected for incorporation into a preparation of uncultured fecal bacteria. In an aspect, the selection of fecal bacteria comprises selecting all of the bacteria extracted from a stool or portion thereof of a donor, based on an ability of fecal bacteria of a stool of the same donor to produce one or more SCFAs. In an aspect, the selection of fecal bacteria comprises selecting a complete or substantially complete fecal microbiota of a stool or portion thereof of a donor, based on an ability of fecal bacteria of stool of the same donor to produce one or more SCFAs. It will therefore be understood that the “unit of selection” when selecting fecal bacteria as described herein is typically the fecal bacteria extracted from stool of a donor, which are obtained (i.e. selected) on the basis of an ability of bacteria from stool of the same donor to produce one or more SCFAs (e.g., at or above a threshold level). Typically selection of fecal bacteria as understood herein does not comprise removing or selecting out particular strains of bacteria from the totality of extracted fecal bacteria after harvesting the bacteria from the stool.

Provided herein are pharmaceutical compositions comprising fecal bacteria (e.g., uncultured fecal bacteria) selected to produce one or more SCFAs in an intestine of a subject administered the composition. In aspects, the fecal bacteria selected to produce one or more SCFAs in an intestine of a subject comprises Faecalibacterim prausnitzii. In another aspect, the fecal bacteria selected to produce one or more SCFAs in an intestine of a subject comprises Eubacterium rectale. In another aspect, the fecal bacteria selected to produce one or more SCFAs in an intestine of a subject comprises Faecalibacterim prausnitzii and Eubacterium rectale. In yet another aspect, the fecal bacteria selected to produce one or more SCFAs in an intestine of a subject comprises one or more bacteria selected from the group consisting of Eubacterium rectale, Roseburia intestinalis, Roseburia faecis, Roseburia hominis, Roseburia inulinivorans, Butyrivibrio fibrisolvens, Eubacterium ramulus, Eubacterim hallii, Anaerostipes caccae, Coprococcus catus GD/7, Coprococcus eutactus L2-50, Coprococcus comes A2-232, Eubacterium cylindroides, Faecalibacterium prausnitzii, Subdoligranulum variabile, and Anaerotruncus colihominis. In another aspect, the fecal bacteria selected to produce one or more SCFAs in an intestine of a subject comprises a bacteria species of Clostridium XIVa and Clostridium IV clusters. The present disclosure contemplates that any method, assay, test or protocol can be used to select for uncultured fecal bacteria capable of producing one or more SCFAs (e.g., at or above a threshold level). For example, in an aspect, a method comprises determining the presence or an amount of one or more SCFAs in stool of a donor (i.e., harboring fecal bacteria), extracting fecal bacteria from stool of the donor (e.g., the same stool or a different stool) to produce a preparation of uncultured fecal bacteria, and incorporating the preparation of uncultured fecal bacteria into the pharmaceutical composition based on the presence or the amount of the one or more SCFAs in the stool. In another aspect, a method comprises extracting fecal bacteria from stool of a donor, directly determining an ability or potential of the fecal bacteria to produce one or more SCFAs (e.g., at or above a threshold level), and incorporating a preparation of uncultured fecal bacteria from stool of the donor (e.g., the same stool or a different stool) into a pharmaceutical composition based on the ability of the fecal bacteria to produce the one or more SCFAs. In another aspect, a method comprises determining a relative abundance of one or more SCFA-producing bacterial strains in stool of a donor, and incorporating a preparation of uncultured fecal bacteria from stool of the donor (e.g., the same stool or a different stool) into a pharmaceutical composition based on the relative abundance of the one or more SCFA-producing bacterial strains.

In an aspect, a method comprises determining the presence or an amount of one or more SCFAs in stool of a donor, extracting fecal bacteria from stool of the donor (e.g., the same stool or a different stool) to produce a preparation of uncultured fecal bacteria, and incorporating the preparation of uncultured fecal bacteria into the pharmaceutical composition based on the presence or the amount of the one or more SCFAs in the stool. In this aspect, an ability of fecal bacteria of a stool to produce an SCFA is inferred based on a level of the SCFA in the stool. In an aspect, a level of SCFA is determined by directly quantifying the SCFA from the stool of the donor. Any method known in the art can be used to determine or quantify an amount of an SCFA from a stool. For example, a sample of raw stool (e.g., about 250 mg) can be mixed with water and centrifuged. A volume of the supernatant (e.g., 100 μl) can then be mixed with sulfuric acid and diethyl ether and centrifuged, and the SCFA measured using gas chromatography (e.g., GC-FID).

In various aspects, fecal bacteria of stool of a donor are selected for incorporation into a pharmaceutical composition on the basis of a determination that butyrate in stool of the donor is at or above a threshold level. In aspects, the threshold level of butyrate in the stool of a donor (e.g., at or above which level fecal bacteria of stool of the donor can be incorporated into a pharmaceutical composition) is 10, 15, 20, 25, 30, 35, 40, 45, 50, 55, 60, 65, 70, 75, 80, or greater than 80 nmol per gram of fresh or raw stool.

In various aspects, fecal bacteria of stool of a donor are selected for incorporation into a pharmaceutical composition on the basis of a determination of the abundance of a butyrate-producing bacterium. In an aspect, stool of a donor are selected for incorporation into a pharmaceutical composition on the basis of a determination of the abundance of one or more, two or more, three or more, four or more, five or more, or six or more butyrate-producing bacterium selected from the group consisting of Eubacterium rectale, Roseburia intestinalis, Roseburia faecis, Roseburia hominis, Roseburia inulinivorans, Butyrivibrio fibrisolvens, Eubacterium ramulus, Eubacterim hallii, Anaerostipes caccae, Coprococcus catus GD/7, Coprococcus eutactus L2-50, Coprococcus comes A2-232, Eubacterium cylindroides, Faecalibacterium prausnitzii, Subdoligranulum variabile, and Anaerotruncus colihominis.

In an aspect, fecal bacteria of a stool of a donor are selected based on the difference in butyrate levels between stool of donors and pre-FMT patients in need thereof. In another aspect, a donor is selected based on the difference in butyrate levels between stool of donors and pre-FMT patients in need thereof. In an aspect, the difference in butyrate levels between stool of donors and pre-FMT patients is at least a 2-, 3-, 4-, 5-, 6-, 7-, 8-, 9, or 10-fold difference. In another aspect, the difference in butyrate levels between stool of donors and pre-FMT patients is at least a 10, 20, 30, 40, 50, 60, 70, 80, or 90% difference. In an aspect, the difference in butyrate levels between stool of donors and pre-FMT patients is between 2- and 4-fold, 5- and 7-fold, 8- and 10-fold, 1.5 and 10-fold, or 2- and 10-fold difference. In another aspect, the difference in butyrate levels between stool of donors and pre-FMT patients is between 10 and 20%, 20 and 30%, 30 and 40%, 40 and 50%, 50 and 60%, 60 and 70%, 70 and 80%, 80 and 90%, 10 and 30%, 30 and 70%, or 70 and 100% difference. In another aspect, the difference in butyrate levels between stool of donors and pre-FMT patients is 10, 15, 20, 25, 30, 35, 40, 45, 50, 55, 60, 65, 70, 75, 80, or greater than 80 nmol per gram of fresh or raw stool. In another aspect, the differences in butyrate levels between stool of donors and pre-FMT patients is measured by an in stool or ex vivo assay.

In various aspects, fecal bacteria of stool of a donor are selected for incorporation into a pharmaceutical composition on the basis of a determination that acetate in stool of the donor is at or above a threshold level. In aspects, the threshold level of acetate in the stool of a donor (e.g., at or above which level which fecal bacteria of stool of the donor can be incorporated into a pharmaceutical composition) is 10, 15, 20, 25, 30, 35, 40, 45, 50, 55, 60, 65, 70, 75, 80, 85, 90, 95, 100, 105, 110, 115, 120, 125, 130, 135, 140, 145, 150, or greater than 150 nmol per gram of fresh or raw stool.

In various aspects, fecal bacteria of stool of a donor are selected for incorporation into a pharmaceutical composition on the basis of a determination that caproate in stool of the donor is at or above a threshold level. In aspects, the threshold level of caproate in the stool of a donor (e.g., at or above which level fecal bacteria of stool of the donor can be incorporated into a pharmaceutical composition) is 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15 or greater than 15 nmol per gram of fresh or raw stool.

In various aspects, fecal bacteria of stool of a donor are selected for incorporation into a pharmaceutical composition on the basis of a determination that heptanoate in stool of the donor is at or above a threshold level. In aspects, the threshold level of heptanoate in the stool of a donor (e.g., at or above which level fecal bacteria of stool of the donor can be incorporated into a pharmaceutical composition) is 0.2, 0.4, 0.6, 0.8, 1, 1.2 1.4, 1.6, 1.8, 2, 2.2, 2.4, 2.6, 2.8, 3, 3.2, 3.4, 3.6, 3.8, 4, 4.2, 4.4, 4.6, 4.8, 5, or greater than 5 nmol per gram of fresh or raw stool.

In various aspects, fecal bacteria of stool of a donor are selected for incorporation into a pharmaceutical composition on the basis of a determination that isobutyrate in stool of the donor is at or above a threshold level. In aspects, the threshold level of isobutyrate in the stool of a donor (e.g., at or above which level fecal bacteria of stool of the donor can be incorporated into a pharmaceutical composition) is 2, 2.2, 2.4, 2.6, 2.8, 3, 3.2, 3.4, 3.6, 3.8, 4, 4.2, 4.4, 4.6, 4.8, 5, 5.2, 5.4, 5.6, 5.8, 6, 6.2, 6.4, 6.6, 6.8, 7, 7.2, 7.4, 7.6, 7.8, 8, 8.2, 8.4, 8.6, 8.8, 9, 9.2, 9.4, 9.6, 9.8, 10, or greater than 10 nmol per gram of fresh or raw stool.

In various aspects, fecal bacteria of stool of a donor are selected for incorporation into a pharmaceutical composition on the basis of a determination that isocaproate in stool of the donor is at or above a threshold level. In aspects, the threshold level of isocaproate in the stool of a donor (e.g., at or above which level fecal bacteria of stool of the donor can be incorporated into a pharmaceutical composition) is 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, or greater than 2 nmol per gram of fresh or raw stool.

In various aspects, fecal bacteria of stool of a donor are selected for incorporation into a pharmaceutical composition on the basis of a determination that isovalerate in stool of the donor is at or above a threshold level. In aspects, the threshold level of isovalerate in the stool of a donor (e.g., at or above which level fecal bacteria of stool of the donor can be incorporated into a pharmaceutical composition) is 2, 2.2, 2.4, 2.6, 2.8, 3, 3.2, 3.4, 3.6, 3.8, 4, 4.2, 4.4, 4.6, 4.8, 5, 5.2, 5.4, 5.6, 5.8, 6, 6.2, 6.4, 6.6, 6.8, 7, 7.2, 7.4, 7.6, 7.8, 8, 8.2, 8.4, 8.6, 8.8, 9, 9.2, 9.4, 9.6, 9.8, 10, or greater than 10 nmol per gram of fresh or raw stool.

In various aspects, fecal bacteria of stool of a donor are selected for incorporation into a pharmaceutical composition on the basis of a determination that propionate in stool of the donor is at or above a threshold level. In aspects, the threshold level of propionate in the stool of a donor (e.g., at or above which level fecal bacteria of stool of the donor can be incorporated into a pharmaceutical composition) is 10, 15, 20, 25, 30, 35, 40, 45, 50, 55, 60, 65, 70, 75, 80, 85, 90, or greater than 90 nmol per gram of fresh or raw stool.

In various aspects, fecal bacteria of stool of a donor are selected for incorporation into a pharmaceutical composition on the basis of a determination that valerate in stool of the donor is at or above a threshold level. In aspects, the threshold level of valerate in the stool of a donor (e.g., at or above which level fecal bacteria of stool of the donor can be incorporated into a pharmaceutical composition) is 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15 or greater than 15 nmol per gram of fresh or raw stool.

In an aspect, a method comprises directly determining an ability or potential of fecal bacteria from stool of a donor to produce one or more SCFAs (e.g., at or above a threshold level), and incorporating a preparation of uncultured fecal bacteria prepared from stool of the same donor (e.g., the same stool or a different stool) into a pharmaceutical composition based on the ability or potential of the fecal bacteria to produce the one or more SCFAs. In this aspect, intestinal bacteria are screened or assayed in real time for their ability to produce an SCFA by metabolizing one or more substrates during an incubation period. Any functional assay or ex vivo assay known to a person skilled in the art can be used to assay the bacteria for their ability to produce the SCFA. Typically in such functional assays the fecal bacteria are at least partially purified, extracted or harvested from non-bacterial stool matter (e.g., fiber) prior to incubating the bacteria with a substrate. This method for screening fecal bacteria for an ability to produce SCFA is advantageous in that it identifies a source of fecal bacteria (e.g., the stool from which a sample of bacteria is extracted) known to be viable and functionally capable of generating an SCFA. Bacterial cells from a remaining portion of the stool (i.e., not used in performing the assay), or from a different stool of the same donor, can then be incorporated into a preparation of uncultured fecal bacteria for incorporation into a pharmaceutical composition. In a non-limiting example of an ex vivo assay, a sample of raw stool from a donor (e.g., about 10 ml) can be homogenized and centrifuged, the pellet resuspended in buffer (e.g., sodium phosphate buffer), and the fecal bacteria incubated with one or more substrates that can be metabolized by fecal bacteria to produce SCFA. Non-limiting examples of substrates that can be used in the functional assay include an amino acid (e.g., valine, leucine, isoleucine), lactic acid, ammonium nitrate, amylose, barley mulch, biotin, carbonate, cellulose, chitin, choline, fructooligosaccharides (FOSs), fructose, glucose, glycerol, heteropolysaccharide, histidine, homopolysaccharide, hydroxyapatite, inulin, isomaltulose, lactose, lactulose, maltodextrins, maltose, nitrogen, oligodextrose, oligofructose, oligofructose-enriched inulin, an oligosaccharide (e.g. comprising a galactooligosaccharide (GOS), trans-galactooligosaccharide, fructooligosaccharide (FOS), xylooligosaccharides (XOS), mannooligosaccharide, or chitooligosaccharide), pectin, phosphate salts, phosphorus, polydextroses, polyols, potash, potassium, sodium nitrate, starch, sucrose, sulfur, sun fiber, tagatose, thiamine, trehalose, vitamins, a water-soluble carbohydrate, a fermentable polysaccharide, a dietary fiber, resistant starch, barley, white navy bean powder, or a combination thereof. The fecal bacteria can be incubated with the substrate for any duration of time sufficient to allow production of the SCFA by the bacteria, for example at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, or greater than 24 hours.

In various aspects, fecal bacteria of stool of a donor are selected for incorporation into a pharmaceutical composition on the basis of a determination (e.g., using a functional assay) that fecal bacteria of stool of the donor are capable of producing butyrate at or above a threshold level. In an aspect, the threshold level of butyrate produced in the functional assay (i.e., at or above which level fecal bacteria of stool of the donor can be incorporated into a pharmaceutical composition) can be at least 0.5, at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 10.5, at least 11, at least 11.5, at least 12, at least 12.5, at least 13, at least 13.5, at least 14, at least 14.5, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, or greater than 200 mM of butyrate.

In various aspects, fecal bacteria of stool of a donor are selected for incorporation into a pharmaceutical composition on the basis of a determination (e.g., using a functional assay) that fecal bacteria of stool of the donor are capable of producing acetate at or above a threshold level. In an aspect, the threshold level of acetate produced in the functional assay (i.e., at or above which level fecal bacteria of stool of the donor can be incorporated into a pharmaceutical composition) can be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, or greater than 200 mM of acetate.

In various aspects, fecal bacteria of stool of a donor are selected for incorporation into a pharmaceutical composition on the basis of a determination (e.g., using a functional assay) that fecal bacteria of stool of the donor are capable of producing propionate at or above a threshold level. In an aspect, the threshold level of propionate produced in the functional assay (i.e., at or above which level fecal bacteria of stool of the donor can be incorporated into a pharmaceutical composition) can be at least 0.5, at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 10.5, at least 11, at least 11.5, at least 12, at least 12.5, at least 13, at least 13.5, at least 14, at least 14.5, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100, or greater than 100 mM of propionate.

In various aspects, fecal bacteria of stool of a donor are selected for incorporation into a pharmaceutical composition on the basis of a determination (e.g., using a functional assay) that fecal bacteria of stool of the donor are capable of producing one or more of caproate, heptanoate, isobutyrate, isocaproate, isovalerate, or valerate at or above a threshold level. In an aspect, the threshold level of caproate, heptanoate, isobutyrate, isocaproate, isovalerate, or valerate produced in the functional assay (i.e., at or above which level fecal bacteria of stool of the donor can be incorporated into a pharmaceutical composition) can be at least 0.2, at least 0.4, at least 0.6, at least 0.8, at least 1, at least 1.2, at least 1.4, at least 1.6, at least 1.8, at least 2, at least 2.2, at least 2.4, at least 2.6, at least 2.8, at least 3, at least 3.2, at least 3.4, at least 3.6, at least 3.8, at least 4, at least 4.5, at least 5, at least 5.5, at least 6, at least 6.5, at least 7, at least 7.5, at least 8, at least 8.5, at least 9, at least 9.5 at least 10, or greater than 10 mM.

In another aspect, a method comprises determining a relative abundance of one or more SCFA-producing bacterial strains in stool of a donor, and incorporating a preparation of uncultured fecal bacteria from stool of the donor (e.g., the same stool or a different stool) into a pharmaceutical composition based on the relative abundance of the one or more SCFA-producing bacterial strains.

In an aspect, a preparation of uncultured fecal bacteria is prepared from the same stool that is assayed to determine a suitability of fecal bacteria of the stool for incorporation into a composition described herein (e.g., directly by quantification of the SCFA in the stool, via a functional assay that determines the level of SCFA produced by fecal bacteria from the stool, or by determining a relative abundance of one or more SCFA-producing bacterial strains in the stool). For example, a stool received from a donor can be divided into two or more portions, of which a first portion can be subjected to an assay for determining an ability of fecal bacteria of the stool to produce one or more SCFAs (e.g., via direct quantification of the SCFA, via a functional assay, or by identifying one or more SCFA-producing bacterial strains), and a second portion (e.g., the remaining stool) can be frozen until the determination is made. If the fecal bacteria of the first portion are determined to be capable of producing one or more SCFAs (e.g., by determining a level of the one or more SCFAs in the first portion to be at or above a threshold level, by determining a level of the one or more SCFAs produced by the fecal bacteria in a functional assay to be at or above a threshold level, or by determining a relative abundance of one or more SCFA-producing bacterial strains in the first portion to be at or above a threshold level), then the stored stool can be thawed and processed to produce a preparation of uncultured fecal bacteria for incorporation into a pharmaceutical composition. In another aspect, a second portion (e.g., the remaining stool) of the stool can be processed without freezing to produce a preparation of uncultured fecal bacteria. The preparation can then be selected for incorporation into a pharmaceutical composition based on determining that the fecal bacteria of the first portion are capable of producing one or more SCFAs (e.g., by determining a level of the one or more SCFAs in the first portion to be at or above a threshold level, by determining a level of the one or more SCFAs produced by the fecal bacteria in a functional assay to be at or above a threshold level, or by determining a relative abundance of one or more SCFA-producing bacterial strains in the first portion to be at or above a threshold level).

In another aspect, a preparation of uncultured fecal bacteria can be prepared from stool that is different than the stool used to determine a suitability of the fecal bacteria for incorporation into a pharmaceutical composition (e.g., directly by quantifying one or more SCFAs in the stool, via a functional assay that determines the level of one or more SCFAs produced by fecal bacteria from the stool, or by determining a relative abundance of one or more SCFA-producing bacterial strains in the stool), but is from the same donor. For example, a first stool (or a sample thereof) received from a donor can be subjected to an assay for determining an ability of fecal bacteria of the first stool to produce one or more SCFAs (e.g., via direct quantification of the SCFA, via a functional assay, or by determining a relative abundance of one or more SCFA-producing bacterial strains), and a second stool (or a sample thereof) received from the same donor can be processed to produce a preparation of uncultured fecal bacteria for incorporation into a pharmaceutical composition based on a determination that the fecal bacteria of the first stool can produce one or more SCFAs (e.g., by determining a level of the one or more SCFAs in the first stool to be at or above a threshold level, by determining a level of the one or more SCFAs produced by the fecal bacteria in a functional assay to be at or above a threshold level, or by determining a relative abundance of one or more SCFA-producing bacterial strains in the first stool to be at or above a threshold level). In this aspect, where a donor consistently donates stool having fecal bacteria that can produce one or more SCFAs (e.g., at or above a threshold level), the donor's stool can be used as a source of uncultured fecal bacteria without having to test every donation received from the donor.

Disclosed herein is a method of selecting a stool donor (or a method of selecting stool of a donor for producing a preparation of uncultured fecal bacteria) comprising determining that fecal bacteria of stool of the donor can produce one or more SCFAs (e.g., at or above a threshold level), selecting stool of the donor (e.g., the same stool or different stool) based on the determination that the fecal bacteria can produce one or more SCFAs, and incorporating fecal bacteria of the selected stool into a pharmaceutical composition (e.g., as a preparation of uncultured fecal bacteria). For example, the donor can be selected on the basis of determining that one or more SCFAs in the stool of the donor are at a level at or above a threshold level, by determining via a functional assay that fecal bacteria from stool of the donor produce one or more SCFAs at a level at or above a threshold level, or by determining a relative abundance of one or more SCFA-producing bacterial strains in stool of the donor is at or above a threshold level.

In an aspect, a method of selecting a human stool donor (or a method of selecting stool of a donor for producing a preparation of uncultured fecal bacteria) comprises determining that fecal bacteria of a plurality of stools of the donor can produce one or more SCFAs, for example by determining that an SCFA is produced at or above a threshold level (e.g., in an ex vivo assay or by directly quantifying from stool) in multiple stools or by determining that a relative abundance of one or more SCFA-producing bacterial strains in multiple stools is at or above threshold level. In aspects, stool from the donor can be collected at different time points, such as 2, 3, 4, 5, 6, 7, 8, 9, 10, 11, 12, 13, 14, 15, 16, 17, 18, 19, 20, or greater than 20 time points, and the ability of fecal bacteria in each stool to produce one or more SCFAs determined at each time point. In an aspect, the plurality of stools collected from the donor are from successive bowel movements of the donor. In an aspect, the plurality of stools collected from the donor are collected on the same day. In an aspect, the plurality of stools collected from the donor are collected on different days. In an aspect, at least two of the plurality of stools collected from the donor are collected at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or greater than 24 hours apart. In an aspect, at least two of the plurality of stools collected from the donor are collected at least 1, at least 2, at least 3, at least 4, at least 5, at least 6, at least 7, or greater than 7 days apart. In an aspect, at least two of the plurality of stools collected from the donor are collected at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or greater than 20 weeks apart. In an aspect, at least two of the plurality of stools collected from the donor are collected at least 1, at least 2, at least 3, at least 4, at least 5, at least 6, at least 7, at least 8, at least 9, at least 10, at least 11, at least 12, or greater than 12 months apart.

Provided herein is a method of selecting a stool donor (or a method of selecting stool of a donor for producing a preparation of uncultured fecal bacteria) comprising determining that fecal bacteria from a first stool of an individual produce one or more SCFAs at or above a threshold level, determining that fecal bacteria of a second stool (e.g., collected at a different time point) of the individual produce one or more SCFAs at or above a threshold level, and selecting the individual as the stool donor based on determining that fecal bacteria from the first and the second stools produce the one or more SCFAs at or above the threshold level. The method can further comprise extracting fecal bacteria from stool of the selected donor for incorporating into a pharmaceutical composition (e.g., as a preparation of uncultured fecal bacteria).

Further provided herein is a method of selecting a stool donor (or a method of selecting stool of a donor for producing a preparation of uncultured fecal bacteria) comprising determining that a relative abundance of one or more SCFA-producing bacterial strains in a first stool of an individual is at or above a threshold level, determining that a relative abundance of one or more SCFA-producing bacterial strains in a second stool of the donor is at or above a threshold level, and selecting the individual as the stool donor based on determining that the relative abundance of the one or more SCFA-producing bacterial strains in the first and second stools is at or above the threshold level. The method can further comprise extracting fecal bacteria from stool of the selected donor for incorporating into a pharmaceutical composition (e.g., as a preparation of uncultured fecal bacteria).

In an aspect, the donor is selected based on determining from at least 2, at least 3, at least 4, at least 5, at least 6, at least 7, at least 8 at least 9, at least 10, at least 11, at least 12, at least 13, at least 14, at least 15, or greater than 15 stools that fecal bacteria of each stool produce one or more SCFAs, for example by determining that the fecal bacteria produce the SCFA at or above a threshold level (e.g., as quantified from raw stool or in an ex vivo assay), or by determining that a relative abundance of one or more SCFA-producing bacterial strains is at or above a threshold level. In an aspect, once a stool donor is selected, stool of the donor can be collected and fecal bacteria from the stool used to produce a preparation of uncultured fecal bacteria for incorporation into a pharmaceutical composition described herein.

In another aspect, prior to making a fecal donation, a stool donor can ingest one or more bacterial isolates (e.g., one or more SCFA-producing bacterial isolates or a bile acid transforming isolate), for example in the form of one or more probiotics. In an aspect, a stool donor can ingest a bacterial isolate prior to donating a stool in order to introduce the bacterial isolate into fecal bacteria of the donated stool, i.e. as a bacterial strain (e.g., SCFA-producing bacterial strain or a bile acid transforming isolate). Therefore, a bacterial isolate desirable for inclusion in a bacterial mixture of a pharmaceutical composition can be introduced into the fecal bacteria of a stool donor via ingestion of the bacterial isolate by the donor, thereby allowing a preparation of uncultured fecal bacteria to be prepared from the stool of the donor that is “ready-made” with, or already includes, a bacterial strain (e.g., SCFA-producing bacterial strain or a bile acid transforming isolate) originating from the desired bacterial isolate. A preparation of uncultured fecal bacteria prepared from the stool of a donor that has ingested a bacterial isolate (e.g., in the form of a probiotic) can be directly incorporated into a pharmaceutical composition described herein, without adding any additional bacterial isolate to the preparation, or alternatively can be further spiked or enriched with an additional dose of the bacterial isolate. Such “pre-spiking” of a stool donor's fecal microbiota with one or more desired bacterial strains originating as bacterial isolates dosed to a stool donor can be especially advantageous where a fecal microbiota of the donor does not endogenously comprise a bacterial strain of the same taxonomic category as the bacterial isolate (e.g., phylum, class, order, family, genus or species), or does not endogenously comprise a bacterial strain having a genetic identity to the bacterial isolate that is above a threshold level (e.g., having a 16S rRNA sequence with greater than 97% identity, greater than 98% identity, greater than 99% identity, greater than 99.1% identity, greater than 99.2% identity, greater than 99.3% identity, greater than 99.4% identity, greater than 99.5% identity, greater than 99.6% identity, greater than 99.7% identity, greater than 99.8% identity, or greater than 99.9% identity to a 16S rRNA sequence of the bacterial isolate). Herein a bacterial isolate incorporated into a fecal microbiota via ingestion of the bacterial isolate by a donor of the fecal microbiota is referred to as a “bacterial strain” (i.e., originating from the ingested bacterial isolate) to distinguish it from the purified bacterial isolate existing ex vivo.

In an aspect, a donor can ingest a probiotic comprising a bacterial isolate (e.g., an SCFA-producing bacterial isolate or a bile acid transforming isolate) of a taxonomic category that is not detectable, or is present at a relative abundance below a threshold abundance, in a stool of the donor prior to ingestion of the probiotic. For example, the microbiota of a stool of a donor can be screened (e.g. using a nucleic acid hybridization technique such as PCR) for the presence of a particular taxa (phylum, class, order, family, genus, species or strain) in the fecal microbiota of the donor. If the taxa either is not found in the fecal microbiota, or is present at a relative abundance below a threshold abundance, then the donor can be administered or ingest a probiotic comprising a bacterial isolate of that taxa.

In an aspect, a duration of time between ingestion of one or more bacterial isolates by a stool donor and collection of a stool from the donor (i.e., comprising a bacterial strain originating from the one or more bacterial isolates) can vary; for example the duration can be at least 1 hour, at least 2 hours, at least 4 hours, at least 6 hours, at least 8 hours, at least 10 hours, at least 12 hours, at least 14 hours, at least 16 hours, at least 18 hours, at least 20 hours, at least 22 hours, at least 24 hours, at least 26 hours, at least 28 hours, at least 30 hours, at least 32 hours, at least 34 hours, at least 36 hours, at least 38 hours, at least 40 hours, at least 42 hours, at least 44 hours, at least 46 hours, at least 48 hours, at least 50 hours, at least 52 hours, at least 54 hours, at least 56 hours, at least 58 hours, at least 60 hours, at least 62 hours, at least 64 hours, at least 66 hours, at least 68 hours, at least 70 hours, at least 72 hours, or greater than 72 hours.

In an aspect, a donor can ingest a single or multiple doses of a bacterial isolate to facilitate the incorporation of the bacterial isolate into a fecal microbiota of the donor as a bacterial strain. In one aspect, a dose of a bacterial isolate can be ingested by the donor at least once or twice daily for at least three consecutive days or weeks. In another aspect, a dose is ingested at least once, twice, or three times daily for a period between 1 and 16 weeks, between 2 and 16 weeks, between 3 and 16 weeks, between 4 and 16 weeks, between 5 and 16 weeks, between 6 and 16 weeks, between 7 and 16 weeks, between 8 and 16 weeks, between 10 and 16 weeks, between 12 and 16 weeks, between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

Disclosed herein is a pharmaceutical composition comprising a preparation of fecal bacteria from a stool of a healthy human donor. In an aspect, the fecal bacteria comprises uncultured bacteria. In another aspect, the fecal bacteria comprises bile acid transforming strains. In another aspect, the fecal bacteria are selected to include at least one bile acid transforming bacterial strain. In yet another aspect, the preparation of fecal bacteria comprises a quantity of secondary bile acids. In another aspect, the fecal bacteria comprises lyophilized bacteria. In yet another aspect, the quantity of secondary bile acid is at or above a predetermined threshold level. In an aspect, the predetermined threshold level is at least 100 μM. In another aspect, the threshold level is 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, or 200 μM. In yet another aspect, the predetermined threshold level is between 5 μM and 20 μM, 5 μM and 50 μM, 5 μM and 100 μM, and 5 μM and 150 μM, 5 μM and 200 μM, 5 μM and 800 μM, 10 μM and 20 μM, 10 μM and 50 μM, 10 μM and 100 μM, 10 μM and 150 μM, 10 μM and 200 μM, 50 μM and 100 μM, 50 μM and 200 μM, 100 μM and 200 μM, or 200 μM and 800 μM. In a further aspect, the preparation of fecal bacteria further comprises an SCFA.

Disclosed herein is a method of selecting a stool of a donor for producing a preparation of uncultured fecal bacteria comprising determining the relative abundance of secondary bile acid in a first stool of an individual is at or above a threshold level, determining that a relative abundance of the secondary bile acid in a second stool of a donor is at or above a threshold level, and selecting the individual as the stool donor based on determining that the relative abundance of the secondary bile acid in the first and second stools is at or above the threshold level. In another aspect, a method of selecting a stool of a donor for producing a preparation of uncultured fecal bacteria comprises determining the relative abundance of secondary bile acid in a stool of an individual and selecting the individual as a stool donor if the relative abundance of the secondary bile acid is at or above a threshold level. In a further aspect, a method of selecting a stool of a donor for producing a preparation of fecal bacteria comprises determining the relative abundance of bacteria capable of transforming primary bile acids to secondary bile acids and selecting the stool if the relative abundance of the secondary bile acids is at or above the threshold level. In yet another aspect, the present disclosure provides for a method of selecting a desired donor by screening potential donors' fecal microbial gene content associated with bile acid transforming strains.

Disclosed herein is a method of manufacturing a pharmaceutical composition, the method comprising: producing a preparation of uncultured fecal bacteria from fecal bacteria of stool of a healthy human donor; and formulating the preparation of uncultured fecal bacteria as the pharmaceutical composition, wherein the preparation of uncultured fecal bacteria comprises a bacterial strain originating from a bacterial isolate (e.g., an SCFA-producing bacterial isolate or a bile acid transforming isolate) ingested by the healthy human donor.

It will be appreciated that compositions, dosage forms, and medicaments as described herein include combination pharmaceutical compositions in which one or more additional compounds or medications are added to or otherwise co-administered with a purified fecal microbiota composition.

In an aspect, the present disclosure provides for the following embodiments:

Embodiment 1. A pharmaceutical composition comprising a preparation of fecal bacteria from a stool of a healthy human donor, wherein the fecal bacteria are selected to produce at least one short chain fatty acid (SCFA), wherein the preparation of fecal bacteria comprises uncultured bacteria, and wherein the preparation of fecal bacteria comprises lyophilized bacteria.

Embodiment 2. The pharmaceutical composition of embodiment 1, wherein the at least one SCFA is selected from the group consisting of butyrate, acetate, caproate, heptanoate, isobutyrate, isocaproate, isovalerate, propionate, valerate, and a combination thereof.

Embodiment 3. The pharmaceutical composition of any one of embodiment 1 or embodiment 2, wherein the fecal bacteria are selected to produce the at least one SCFA at a level that is at or above a threshold level.

Embodiment 4. The pharmaceutical composition of embodiment 3, wherein the fecal bacteria are selected using a functional assay for determining the level of the at least one SCFA.

Embodiment 5. The pharmaceutical composition of embodiment 4, wherein the functional assay comprises an ex vivo assay.

Embodiment 6. The pharmaceutical composition of embodiment 5, wherein the ex vivo assay comprises contacting the fecal bacteria with a substrate and determining an amount of the at least one SCFA produced by the fecal bacteria by metabolizing the substrate.

Embodiment 7. The pharmaceutical composition of embodiment 6, wherein the substrate is selected from the group consisting of inulin, fructooligosaccharide, sunfiber, white navy bean powder, and a combination thereof.

Embodiment 8. The pharmaceutical composition of embodiment 6 or embodiment 7, wherein the fecal bacteria are contacted with the substrate for at least 5 hours.

Embodiment 9. The pharmaceutical composition of embodiment 6 or embodiment 7, wherein the fecal bacteria are contacted with the substrate for at least 10 hours.

Embodiment 10. The pharmaceutical composition of any one of embodiments 3 to 9, wherein the at least one SCFA comprises butyrate.

Embodiment 11. The pharmaceutical composition of embodiment 10, wherein the threshold level of butyrate is 20 mM.

Embodiment 12. The pharmaceutical composition of embodiment 10, wherein the threshold level of butyrate is 40 mM.

Embodiment 13. The pharmaceutical composition of embodiment 10, wherein the threshold level of butyrate is 60 mM.

Embodiment 14. The pharmaceutical composition of embodiment 10, wherein the threshold level of butyrate is 80 mM.

Embodiment 15. The pharmaceutical composition of any one of embodiments 3 to 9, wherein the at least one SCFA is acetate.

Embodiment 16. The pharmaceutical composition of embodiment 15, wherein the threshold level of acetate is 20 mM.

Embodiment 17. The pharmaceutical composition of embodiment 15, wherein the threshold level of acetate is 40 mM.

Embodiment 18. The pharmaceutical composition of embodiment 15, wherein the threshold level of acetate is 60 mM.

Embodiment 19. The pharmaceutical composition of embodiment 15, wherein the threshold level of acetate is 80 mM.

Embodiment 20. The pharmaceutical composition of embodiment 15, wherein the threshold level of acetate is 100 mM.

Embodiment 21. The pharmaceutical composition of any one of embodiments 3 to 9, wherein the at least one SCFA is propionate.

Embodiment 22. The pharmaceutical composition of embodiment 21, wherein the threshold level of propionate is 10 mM.

Embodiment 23. The pharmaceutical composition of embodiment 21, wherein the threshold level of propionate is 15 mM.

Embodiment 24. The pharmaceutical composition of embodiment 21, wherein the threshold level of propionate is 20 mM.

Embodiment 25. The pharmaceutical composition of embodiment 21, wherein the threshold level of propionate is 25 mM.

Embodiment 26. The pharmaceutical composition of embodiment 3, wherein the fecal bacteria are selected by determining the content of the at least one SCFA from raw stool of the donor.

Embodiment 27. The pharmaceutical composition of embodiment 26, wherein the at least one SCFA is butyrate.

Embodiment 28. The pharmaceutical composition of embodiment 27, wherein the threshold level of butyrate is 25 nM per gram of raw stool.

Embodiment 29. The pharmaceutical composition of embodiment 27, wherein the threshold level of butyrate is 40 nM per gram of raw stool.

Embodiment 30. The pharmaceutical composition of embodiment 27, wherein the threshold level of butyrate is 50 nM per gram of raw stool.

Embodiment 31. The pharmaceutical composition of embodiment 26, wherein the at least one SCFA is acetate.

Embodiment 32. The pharmaceutical composition of embodiment 31, wherein the threshold level of acetate is 50 nM per gram of raw stool.

Embodiment 33. The pharmaceutical composition of embodiment 31, wherein the threshold level of acetate is 75 nM per gram of raw stool.

Embodiment 34. The pharmaceutical composition of embodiment 31 wherein the threshold level of acetate is 90 nM per gram of raw stool.

Embodiment 35. The pharmaceutical composition of embodiment 31, wherein the threshold level of acetate is 100 nM per gram of raw stool.

Embodiment 36. The pharmaceutical composition of embodiment 26, wherein the at least one SCFA is propionate.

Embodiment 37. The pharmaceutical composition of embodiment 36, wherein the threshold level of propionate is 20 nM per gram of raw stool.

Embodiment 38. The pharmaceutical composition of embodiment 36, wherein the threshold level of propionate is 30 nM per gram of raw stool.

Embodiment 39. The pharmaceutical composition of embodiment 36, wherein the threshold level of propionate is 40 nM per gram of raw stool.

Embodiment 40. The pharmaceutical composition of embodiment 1 or embodiment 2, wherein the fecal bacteria are selected on the basis of the relative abundance of one or more SCFA-producing bacterial strains in the stool of the donor.

Embodiment 41. The pharmaceutical composition of embodiment 40, wherein the one or more SCFA-producing bacterial strains is a member of a taxonomic group selected from the group consisting of Firmicutes, Bacteroidetes, Eubacterium sp., Clostridium sp., Faecalibacterium sp., Roseburia sp., Butyrivibrio sp., Anaerostipes sp., Coprococcus sp., Subdoligranulum sp., Anaerotruncus sp., Ruminococcus sp., Eubacterium rectale, Roseburia intestinalis, Roseburia faecis, Roseburia hominis, Roseburia inulinivorans, Roseburia cecicola, Butyrivibrio fibrisolvens, Eubacterium ramulus, Eubacterium hallii, Eubacterium ruminantium, Eubacterium cylindroides, Eubacterium oxidoreducens, Coprococcus catus, Coprococcus eutactus, Coprococcus comes, Faecalibacterium prausnitzii, Subdoligranulum variabile, Anaerotruncus colihominis, Clostridium nexile, Clostridium hathewayi, Clostridium indolis, Clostridium leptum, Ruminococcus gnavus, Ruminococcus obeum, and Anaerostipes caccae.

Embodiment 42. The pharmaceutical composition of any one of embodiments 1 to 41, wherein the composition further comprises an SCFA.

Embodiment 43. The pharmaceutical composition of any one of embodiments 1 to 42, wherein the composition further comprises a prebiotic.

Embodiment 44. The pharmaceutical composition of embodiment 43, wherein the prebiotic is selected from the group consisting of inulin, fructooligosaccharide, sunfiber, white navy bean powder, and a combination thereof.

Embodiment 45. The pharmaceutical composition of any one of embodiments 1 to 44, wherein the preparation of fecal bacteria further comprises a cryoprotectant selected from the group consisting of polyethylene glycol, skim milk, erythritol, arabitol, sorbitol, glucose, fructose, alanine, glycine, proline, sucrose, lactose, ribose, trehalose, dimethyl sulfoxide (DMSO), glycerol, and a combination thereof.

Embodiment 46. The pharmaceutical composition of any one of embodiments 1 to 45, wherein the pharmaceutical composition is formulated for oral administration.

Embodiment 47. The pharmaceutical composition of embodiment 46, wherein the pharmaceutical composition comprises a capsule for encapsulating the preparation of fecal bacteria.

Embodiment 48. The pharmaceutical composition of embodiment 47, wherein the capsule comprises a delayed-release coating.

Embodiment 49. The pharmaceutical composition of any one of embodiments 1 to 48, wherein the fecal bacteria produce the at least one SCFA in an intestine of a subject administered the composition.

Embodiment 50. A method of delivering at least one SCFA to a subject in need thereof comprising administering to the subject the composition of any one of embodiments 1 to 49.

Embodiment 51. A method of treating a gut dysbiosis in a subject in need thereof comprising administering to the subject the composition of any one of embodiments 1 to 49.

Embodiment 52. The method of embodiment 51, wherein the gut dysbiosis is selected from the group consisting of inflammatory bowel disease (IBD), irritable bowel syndrome (IBS), C. difficile infection (CDI), irritable bowel syndrome, and a combination thereof.

Embodiment 53. The method of embodiment 52, wherein the IBD is selected from the group consisting of Crohn's disease, ulcerative colitis, pouchitis, and a combination thereof.

Embodiment 54. A method of delivering a short chain fatty acid (SCFA) to an intestine of a subject in need thereof, the method comprising administering to the subject a pharmaceutical composition comprising a preparation of uncultured fecal bacteria derived from a stool of a healthy human donor, wherein: the uncultured fecal bacteria are selected to produce the SCFA at or above a threshold level, fecal bacteria in a stool of the healthy human donor produce the SCFA at or above a threshold level, or the healthy human donor produces a stool comprising the SCFA at or above a threshold level.

Embodiment 55. The method of embodiment 54, further comprising administering a prebiotic to the subject.

Embodiment 56. The method of embodiment 55, wherein the prebiotic is selected from the group consisting of an amino acid (e.g., valine, leucine, isoleucine), lactic acid, ammonium nitrate, amylose, barley mulch, biotin, carbonate, cellulose, chitin, choline, fructooligosaccharides (FOSs), fructose, glucose, glycerol, heteropolysaccharide, hi stidine, homopolysaccharide, hydroxyapatite, inulin, isomaltulose, lactose, lactulose, maltodextrins, maltose, nitrogen, oligodextrose, oligofructose, oligofructose-enriched inulin, an oligosaccharide (e.g. comprising a galactooligosaccharide (GOS), trans-galactooligosaccharide, fructooligosaccharide (FOS), xylooligosaccharides (XOS), mannooligosaccharide, or chitooligosaccharide), pectin, phosphate salts, phosphorus, polydextroses, polyols, potash, potassium, sodium nitrate, starch, sucrose, sulfur, sun fiber, tagatose, thiamine, trehalose, vitamins, a water-soluble carbohydrate, a fermentable polysaccharide, a dietary fiber, resistant starch, barley, white navy bean powder, and a combination thereof.

Embodiment 57. The method of any one of embodiments 54 to 56, further comprising pretreating the subject with an antibiotic before administering the pharmaceutical composition to the subject.

Embodiment 58. The method of any one of embodiments 54 to 57, wherein the SCFA is selected from the group consisting of butyrate, acetate, caproate, heptanoate, isobutyrate, isocaproate, isovalerate, propionate, valerate, and a combination thereof.

Embodiment 59. The method of any one of embodiments 54 to 58, wherein the fecal bacteria are selected by determining a level of the at least one SCFA in stool of the donor.

Embodiment 60. The method of any one of embodiments 54 to 59, further comprising determining an amount of the SCFA produced by fecal bacteria of the subject to be below a threshold level prior to administering the pharmaceutical composition to the subject.

Embodiment 61. The method of any one of embodiments 54 to 60, wherein administering the pharmaceutical composition treats or prevents a disorder in the subject, wherein the disorder is selected from a C. difficile infection, inflammatory bowel disease, irritable bowel syndrome, and a combination thereof.

Embodiment 62. A method comprising determining a level of at least one SCFA produced by fecal bacteria of a subject; and administering to the subject a pharmaceutical composition based on determining the level of the at least one SCFA to be below a threshold level, wherein the pharmaceutical composition comprises a preparation of uncultured fecal bacteria of stool of a healthy human donor.

Embodiment 63. The method of embodiment 62, wherein the at least one SCFA is selected from the group consisting of butyrate, acetate, caproate, heptanoate, isobutyrate, isocaproate, isovalerate, propionate, valerate, and a combination thereof.

Embodiment 64. The method of embodiment 62 or embodiment 63, wherein the level of the at least one SCFA is determined from raw stool of the subject.

Embodiment 65. The method of any one of embodiments 62 to 64, wherein the at least one SCFA is butyrate.

Embodiment 66. The method of embodiment 65, wherein the threshold level of the butyrate is 2 nM per gram raw stool.

Embodiment 67. The method of embodiment 65, wherein the threshold level of the butyrate is 5 nM per gram raw stool.

Embodiment 68. The method of embodiment 65, wherein the threshold level of the butyrate is 10 nM per gram raw stool.

Embodiment 69. The method of any one of embodiments 62 to 64, wherein the at least one SCFA is acetate.

Embodiment 70. The method of embodiment 65, wherein the threshold level of the acetate is 10 nM per gram raw stool.

Embodiment 71. The method of embodiment 65, wherein the threshold level of the acetate is 25 nM per gram raw stool.

Embodiment 72. The method of embodiment 65, wherein the threshold level of the acetate is 40 nM per gram raw stool.

Embodiment 73. The method of any one of embodiments 62 to 64, wherein the at least one SCFA is propionate.

Embodiment 74. The method of embodiment 73, wherein the threshold level of the propionate is 2 nM per gram raw stool.

Embodiment 75. The method of embodiment 65, wherein the threshold level of the propionate is 8 nM per gram raw stool.

Embodiment 76. The method of embodiment 65, wherein the threshold level of the propionate is 12 nM per gram raw stool.

Embodiment 77. The method of any one of embodiments 62 to 76, wherein the uncultured fecal bacteria are selected to produce the at least one SCFA in the intestine of the subject.

Embodiment 78. The method of embodiment 77, wherein the uncultured fecal bacteria are selected using a functional assay for determining a level of the at least one SCFA.

Embodiment 79. The method of embodiment 78, wherein the functional assay comprises an ex vivo assay.

Embodiment 80. The method of embodiment 79, wherein the ex vivo assay comprises contacting the uncultured fecal bacteria with a substrate and determining a level of the at least one SCFA produced by the uncultured fecal bacteria by metabolizing the substrate.

Embodiment 81. The method of embodiment 80, wherein the substrate is selected from the group consisting of inulin, fructooligosaccharide, sunfiber, white navy bean powder, and a combination thereof.

Embodiment 82. The method of embodiment 77, wherein the uncultured fecal bacteria are selected by determining a level of the at least one SCFA from a stool of the donor.

Embodiment 83. The method of any one of embodiments 62 to 82, wherein administering the pharmaceutical composition increases the level of the at least one SCFA in the intestine of the subject above the threshold level.

Embodiment 84. The method of embodiment 83, wherein administering the pharmaceutical composition increases the level of the at least one SCFA produced by intestinal bacteria of the subject by at least 5%.

Embodiment 85. The method of embodiment 83, wherein administering the pharmaceutical composition increases the level of the at least one SCFA produced by intestinal bacteria of the subject by at least 10%.

Embodiment 86. The method of embodiment 83, wherein administering the pharmaceutical composition increases the level of the at least one SCFA produced by intestinal bacteria of the subject by at least 15%.

Embodiment 87. The method of embodiment 83, wherein administering the pharmaceutical composition increases the level of the at least one SCFA produced by intestinal bacteria of the subject by at least 20%.

Embodiment 88. A method comprising: determining a level of a metabolite produced by fecal bacterial cells to be at or above a threshold level, wherein the fecal bacterial cells are from a stool of a healthy human donor; and extracting fecal bacteria from a stool of the donor based on determining the level of the metabolite to be at or above the threshold level to produce a preparation of uncultured fecal bacteria.

Embodiment 89. The method of embodiment 88, wherein the metabolite is a short-chain fatty acid (SCFA) or a bile acid.

Embodiment 90. The method of embodiment 88 or embodiment 89, wherein the fecal bacterial cells and the fecal bacteria are from the same stool of the donor.

Embodiment 91. The method of embodiment 88 or embodiment 89, wherein the fecal bacterial cells and the fecal bacteria are from different stools of the donor.

Embodiment 92. The method of any one of embodiments 88 to 91, wherein determining the level of the metabolite comprises determining a level of the metabolite in raw stool of the donor.

Embodiment 93. The method of any one of embodiments 88 to 91, wherein determining the level of the metabolite comprises determining a level of the metabolite produced by the fecal bacterial cells in a functional assay.

Embodiment 94. The method of embodiment 93, wherein the functional assay is an ex vivo assay.

Embodiment 95. The method of any one of embodiments 88 to 94, further comprising formulating the preparation of uncultured fecal bacteria as a pharmaceutical composition.

Embodiment 96. A method comprising: determining that a level of a short-chain fatty acid (SCFA) produced by fecal bacterial cells is at or above a threshold level, wherein the fecal bacterial cells are from a stool of a healthy human donor; selecting fecal bacteria of the donor based on determining that the level of the SCFA is at or above the threshold level, wherein the selected fecal bacteria are uncultured; mixing the selected fecal bacteria with a cryoprotectant to produce a preparation of uncultured fecal bacteria.

Embodiment 97. The method of embodiment 96, wherein the fecal bacteria are selected from the stool of the donor used to determine that the level of the SCFA produced by the fecal bacterial cells is at or above the threshold level.

Embodiment 98. The method of embodiment 96, wherein the fecal bacterial cells are selected from a different stool of the donor than the stool used to determine that the level of the SCFA produced by the fecal bacterial cells is at or above the threshold level.

Embodiment 99. The method of any one of embodiment 96 to 98, wherein the at least one SCFA is selected from the group consisting of butyrate, acetate, caproate, heptanoate, isobutyrate, isocaproate, isovalerate, propionate, valerate, and a combination thereof.

Embodiment 100. The method of embodiment 99, wherein determining the level of the SCFA comprises determining a level of the SCFA produced by the fecal bacterial cells in a functional assay.

Embodiment 101. The method of embodiment 100, wherein the functional assay comprises an ex vivo assay.

Embodiment 102. The method of embodiment 101, wherein the ex vivo assay comprises contacting the fecal bacterial cells with a substrate and determining an amount of the SCFA produced by the fecal bacterial cells by metabolizing the substrate.

Embodiment 103. The method of embodiment 102, wherein the substrate is selected from the group consisting of inulin, fructooligosaccharide, sunfiber, white navy bean powder, and a combination thereof.

Embodiment 104. The method of embodiment 102 or embodiment 103, wherein the fecal bacterial cells are contacted with the substrate for at least 5 hours.

Embodiment 105. The method of embodiment 102 or embodiment 103, wherein the fecal bacterial cells are contacted with the substrate for at least 10 hours.

Embodiment 106. The method of any one of embodiments 99 to 105, wherein the SCFA is butyrate.

Embodiment 107. The method of embodiment 106, wherein the threshold level of the butyrate is 20 mM.

Embodiment 108. The method of embodiment 106, wherein the threshold level of the butyrate is 40 mM.

Embodiment 109. The method of embodiment 106, wherein the threshold level of the butyrate is 60 mM.

Embodiment 110. The method of embodiment 106, wherein the threshold level of the butyrate is 80 mM.

Embodiment 111. The method of any one of embodiments 99 to 105, wherein the SCFA is acetate.

Embodiment 112. The method of embodiment 111, wherein the threshold level of the acetate is 40 mM.

Embodiment 113. The method of embodiment 111, wherein the threshold level of the acetate is 60 mM.

Embodiment 114. The method of embodiment 111, wherein the threshold level of the acetate is 80 mM.

Embodiment 115. The method of embodiment 111, wherein the threshold level of the acetate is 100 mM.

Embodiment 116. The method of any one of embodiments 99 to 105, wherein the SCFA is propionate.

Embodiment 117. The method of embodiment 116, wherein the threshold level of the propionate is 10 mM.

Embodiment 118. The method of embodiment 116, wherein the threshold level of the propionate is 15 mM.

Embodiment 119. The method of embodiment 116, wherein the threshold level of the propionate is 20 mM.

Embodiment 120. The method of embodiment 116, wherein the threshold level of the propionate is 25 mM.

Embodiment 121. The method of embodiment 99, wherein determining the level of the SCFA comprises determining a level of the SCFA in raw stool of the donor.

Embodiment 122. The method of embodiment 121, wherein the SCFA is butyrate.

Embodiment 123. The method of embodiment 122, wherein the threshold level of the butyrate is 25 nM per gram of raw stool.

Embodiment 124. The method of embodiment 122, wherein the threshold level of the butyrate is 40 nM per gram of raw stool.

Embodiment 125. The method of embodiment 122, wherein the threshold level of the butyrate is 50 nM per gram of raw stool.

Embodiment 126. The method of embodiment 121, wherein the SCFA is acetate.

Embodiment 127. The method of embodiment 122, wherein the threshold level of the acetate is 50 nM per gram of raw stool.

Embodiment 128. The method of embodiment 122, wherein the threshold level of the acetate is 75 nM per gram of raw stool.

Embodiment 129. The method of embodiment 122, wherein the threshold level of the acetate is 100 nM per gram of raw stool.

Embodiment 130. The method of embodiment 121, wherein the SCFA is propionate.

Embodiment 131. The method of embodiment 122, wherein the threshold level of the propionate is 20 nM per gram of raw stool.

Embodiment 132. The method of embodiment 122, wherein the threshold level of the propionate is 30 nM per gram of raw stool.

Embodiment 133. The method of embodiment 122, wherein the threshold level of the propionate is 40 nM per gram of raw stool.

Embodiment 134. The method of any one of embodiments 96 to 133, wherein the method further comprises extracting the selected fecal bacteria from a stool of the donor prior to mixing the selected fecal bacteria with the cryoprotectant.

Embodiment 135. The method of embodiment 134, wherein the extracting comprises filtering the selected fecal bacteria.

Embodiment 136. The method of any one of embodiments 96 to 135, wherein the cryoprotectant is selected from the group consisting of polyethylene glycol, skim milk, erythritol, arabitol, sorbitol, glucose, fructose, alanine, glycine, proline, sucrose, lactose, ribose, trehalose, dimethyl sulfoxide (DMSO), glycerol, and a combination thereof. Embodiment 137. The method of any one of embodiments 96 to 136, wherein the preparation of uncultured fecal bacteria further comprises an antioxidant.

Embodiment 138. The method of any one of embodiments 96 to 137, wherein the method further comprises encapsulating the preparation of uncultured fecal bacteria in a pharmaceutically acceptable carrier.

Embodiment 139. The method of embodiment 138, wherein the pharmaceutically acceptable carrier comprises a tablet, geltab, pill or capsule.

Embodiment 140. The method of embodiment 139, wherein the pharmaceutically acceptable carrier is a capsule, and the capsule is acid-resistant.

Embodiment 141. A method comprising: determining that a relative abundance of one or more short chain fatty acid (SCFA)-producing bacterial strains is at or above a threshold level in stool of a healthy human donor; selecting fecal bacteria of the donor based on determining that the relative abundance of the one or more SCFA-producing bacterial strains is at or above the threshold level, wherein the selected fecal bacteria are uncultured; and mixing the selected fecal bacteria with a cryoprotectant to produce a preparation of uncultured fecal bacteria.

Embodiment 142. The method of embodiment 141, wherein the fecal bacteria are selected from the stool of the donor used to determine that the relative abundance of one or more SCFA-producing bacterial strains is at or above the threshold level.

Embodiment 143. The method of embodiment 141, wherein the fecal bacteria are selected from a different stool of the donor than the stool used to determine that the relative abundance of one or more SCFA-producing bacterial strains is at or above the threshold level.

Embodiment 144. The method of any one of embodiments 141 to 143, wherein the one or more SCFA-producing bacterial strains is a member of a taxonomic group selected from the group consisting of Firmicutes, Bacteroidetes, Eubacterium sp., Clostridium sp., Faecalibacterium sp., Roseburia sp., Butyrivibrio sp., Anaerostipes sp., Coprococcus sp., Subdoligranulum sp., Anaerotruncus sp., Ruminococcus sp., Eubacterium rectale, Roseburia intestinalis, Roseburia faecis, Roseburia hominis, Roseburia inulinivorans, Roseburia cecicola, Butyrivibrio fibrisolvens, Eubacterium ramulus, Eubacterium hallii, Eubacterium ruminantium, Eubacterium cylindroides, Eubacterium oxidoreducens, Coprococcus catus, Coprococcus eutactus, Coprococcus comes, Faecalibacterium prausnitzii, Subdoligranulum variabile, Anaerotruncus colihominis, Clostridium nexile, Clostridium hathewayi, Clostridium indolis, Clostridium leptum, Ruminococcus gnavus, Ruminococcus obeum, and Anaerostipes caccae.

Embodiment 145. The method of any one of embodiments 141 to 144, wherein the relative abundance of the one or more SCFA-producing bacterial strains is at least 10%.

Embodiment 146. The method of any one of embodiments 141 to 145, wherein the relative abundance of the one or more SCFA-producing bacterial strains is at least 25%.

Embodiment 147. The method of any one of embodiments 141 to 146, wherein the relative abundance of the one or more SCFA-producing bacterial strains is at least 50%.

Embodiment 148. A method comprising: determining from a first stool of a donor that an SCFA in the first stool is at or above a threshold level; determining from a second stool of the donor that the SCFA in the second stool is at or above the threshold level; and extracting fecal bacteria from a stool of the donor to produce a preparation of uncultured fecal bacteria based on determining that the SCFA in the first and second stools is at or above a threshold level.

Embodiment 149. A method comprising: determining in a functional assay that fecal bacterial cells from a first stool of a donor produce an SCFA at or above a threshold level; determining in the functional assay that fecal bacterial cells from a second stool of the donor produce the SCFA at or above the threshold level; and extracting fecal bacteria from a stool of the donor to produce a preparation of uncultured fecal bacteria based on determining that the fecal bacterial cells from the first and second stools produce the SCFA at or above the threshold level in the functional assay.

Embodiment 150. A method comprising: determining from a first stool of a donor that a relative abundance of one or more SCFA-producing bacteria is at or above a threshold level; determining from a second stool of the donor that a relative abundance of one or more SCFA-producing bacteria is at or above the threshold level; and extracting fecal bacteria from a stool of the donor to produce a preparation of uncultured fecal bacteria based on determining that the relative abundance of one or more SCFA-producing bacteria is at or above the threshold level in the first and second stools.

Embodiment 151. The method of embodiment any one of embodiments 148 to 150, wherein extracting fecal bacteria from a stool of the donor comprises extracting the fecal bacteria from at least one of the first and second stools.

Embodiment 152. The method of any one of embodiments 148 to 151, wherein the SCFA is selected from the group consisting of butyrate, acetate, caproate, heptanoate, isobutyrate, isocaproate, isovalerate, propionate, valerate, and a combination thereof.

Embodiment 153. The method of any one of embodiments 148 to 152, wherein the first stool of the donor is collected at a first time point and the second stool of the donor is collected at a second time point.

Embodiment 154. The method of embodiment 153, wherein the first and second time points are separated by at least 24 hours.

Embodiment 155. The method of embodiment 153, wherein the first and second time points are separated by at least 3 days.

Embodiment 156. The method of embodiment 153, wherein the first and second time points are separated by at least 1 week.

Embodiment 157. The method of any one of embodiments 148 to 156, wherein the extracting comprises filtering the fecal bacteria.

Embodiment 158. The method of any one of embodiments 148 to 157, wherein the preparation of uncultured fecal bacteria further comprises a cryoprotectant selected from the group consisting of polyethylene glycol, skim milk, erythritol, arabitol, sorbitol, glucose, fructose, alanine, glycine, proline, sucrose, lactose, ribose, trehalose, dimethyl sulfoxide (DMSO), glycerol, and a combination thereof.

Embodiment 159. The method of any one of embodiments 148 to 158, wherein the preparation of uncultured fecal bacteria further comprises an antioxidant.

Embodiment 160. The method of any one of embodiments 148 to 159, wherein the method further comprises encapsulating the preparation of uncultured fecal bacteria in a pharmaceutically acceptable carrier.

Embodiment 161. The method of embodiment 160, wherein the pharmaceutically acceptable carrier comprises a tablet, geltab, pill or capsule.

Embodiment 162. The method of embodiment 161, wherein the pharmaceutically acceptable carrier is a capsule, and the capsule is acid-resistant.

Embodiment 163. The method of any one of embodiments 88 to 162, further comprising administering a stimulant for one or more SCFA-producing bacterial strains to the human donor.

Embodiment 164. The pharmaceutical composition of any one of embodiments 1 to 49, wherein the stool of the human donor comprises an elevated level of at least one short chain fatty acid (SCFA) from ingestion of a stimulant for one or more SCFA-producing bacterial strains.

Embodiment 165. The pharmaceutical composition of any one of embodiments 1 to 49, wherein the stool of the human donor comprises an elevated level of at least one SCFA-producing bacterial strain from ingestion of a stimulant for one or more SCFA-producing bacterial strains.

Embodiment 166. The pharmaceutical composition of embodiment 164 or 165, wherein the human donor ingests a stimulant for one or more SCFA-producing bacterial strains every day for at least 1, 2, 3, 4, 5, 6, 7, 8, 9, 10, 11, 12, 13, or 14 days prior to donation of the stool.

Embodiment 167. The pharmaceutical composition of embodiment 164 or 165, wherein the human donor ingests a stimulant for one or more SCFA-producing bacterial strains at least 1, 2, 3, 4, 5, 6, 7, 8, 9, 10, 11, 12, 13, or 14 days prior to donation of the stool.

Embodiment 168. The pharmaceutical composition of embodiment Embodiment 64 or 165, wherein the human donor ingests a stimulant for one or more SCFA-producing bacterial strains at least 1, 2, 3, 4, 5, 6, 7, 8, 9, 10, 11, 12, 13, 14, 24, or 48 hours prior to donation of the stool.

Embodiment 169. The pharmaceutical composition of embodiment 164 or 165, wherein the human donor ingests a stimulant for one or more SCFA-producing bacterial strains every day for at least 1, 2, 3, 4, 5, 6, 7, 8, 9, 10, 11, 12, 13, or 14 weeks prior to donation of the stool.

Embodiment 170. The pharmaceutical composition of embodiment 164 or 165, wherein the human donor ingests a stimulant for one or more SCFA-producing bacterial strains every other day for at least 1, 2, 3, 4, 5, 6, 7, 8, 9, 10, 11, 12, 13, or 14 weeks prior to donation of the stool.

Embodiment 171. A pharmaceutical composition comprising a preparation of fecal bacteria from stool of a healthy human donor, wherein the fecal bacteria produce an amount of butyrate at or above a threshold level of butyrate when incubated with a substrate in a functional assay, wherein the preparation of fecal bacteria comprises uncultured bacteria, and wherein the threshold level of butyrate is at least 40 mM.

Embodiment 172. The pharmaceutical composition of embodiment 171, wherein the threshold level of butyrate is at least 60 mM.

Embodiment 173. The pharmaceutical composition of embodiment 171, wherein the threshold level of butyrate is at least 80 mM.

Embodiment 174. The pharmaceutical composition of any one of embodiments 171 to 173, wherein the substrate is selected from the group consisting of inulin, fructooligosaccharide, sunfiber, white navy bean powder, and a combination thereof.

Embodiment 175. The pharmaceutical composition of embodiment 174, wherein the substrate is white navy bean powder.

Embodiment 176. A pharmaceutical composition comprising a preparation of fecal bacteria from stool of a healthy human donor, wherein the fecal bacteria produce an amount of acetate at or above a threshold level of acetate when incubated with a substrate in a functional assay, wherein the preparation of fecal bacteria comprises uncultured bacteria, and wherein the threshold level of acetate is at least 60 mM.

Embodiment 177. The pharmaceutical composition of embodiment 176, wherein the threshold level of acetate is at least 80 mM.

Embodiment 178. The pharmaceutical composition of embodiment 176, wherein the threshold level of acetate is at least 100 mM.

Embodiment 179. A pharmaceutical composition comprising a preparation of fecal bacteria from stool of a healthy human donor, wherein the fecal bacteria produce an amount of propionate at or above a threshold level of propionate when incubated with a substrate in a functional assay, wherein the preparation of fecal bacteria comprises uncultured bacteria, and wherein the threshold level of propionate is at least 10 mM.

Embodiment 180. The pharmaceutical composition of embodiment 179, wherein the threshold level of propionate is at least 20 mM.

Embodiment 181. The pharmaceutical composition of embodiment 180, wherein the threshold level of acetate is at least 25 mM.

Embodiment 182. The pharmaceutical composition of any one of embodiments 176 to 181, wherein the substrate is selected from the group consisting of inulin, fructooligosaccharide, sunfiber, white navy bean powder, and a combination thereof.

Embodiment 183. The pharmaceutical composition of any one of embodiments 171 to 182, wherein the functional assay comprises an ex vivo assay.

Embodiment 184. The pharmaceutical composition of any one of embodiments 171 to 183, wherein the fecal bacteria are incubated with the substrate for at least 6 hours.

Embodiment 185. The pharmaceutical composition of any one of embodiments 171 to 183, wherein the fecal bacteria are incubated with the substrate for at least 10 hours.

Embodiment 186. A pharmaceutical composition comprising a preparation of fecal bacteria from stool of a healthy human donor, wherein the preparation of fecal bacteria comprises short-chain fatty acid (SCFA)-producing bacterial strains, wherein the SCFA-producing bacterial strains represent at least 40% of the total number of bacterial strains in the preparation of fecal bacteria, and wherein the preparation of fecal bacteria comprises uncultured bacteria.

Embodiment 187. The pharmaceutical composition of embodiment 186, wherein the SCFA-producing bacterial strains represent at least 50% of the total number of bacterial strains in the preparation of fecal bacteria.

Embodiment 188. The pharmaceutical composition of embodiment 186, wherein the SCFA-producing bacterial strains represent at least 60% of the total number of bacterial strains in the preparation of fecal bacteria.

Embodiment 189. The pharmaceutical composition of embodiment 186, wherein the SCFA-producing bacterial strains represent at least 70% of the total number of bacterial strains in the preparation of fecal bacteria.

Embodiment 190. The pharmaceutical composition of any one of embodiments 171 to 189, wherein the composition further comprises a short-chain fatty acid (SCFA).

Embodiment 191. The pharmaceutical composition of any one of embodiments 171 to 190, wherein the composition further comprises a prebiotic.

Embodiment 192. The pharmaceutical composition of embodiment 191, wherein the prebiotic is selected from the group consisting of inulin, fructooligosaccharide, sunfiber, white navy bean powder, and a combination thereof.

Embodiment 193. The pharmaceutical composition of any one of embodiments 171 to 192, wherein the preparation of fecal bacteria further comprises a cryoprotectant selected from the group consisting of polyethylene glycol, skim milk, erythritol, arabitol, sorbitol, glucose, fructose, alanine, glycine, proline, sucrose, lactose, ribose, trehalose, dimethyl sulfoxide (DMSO), glycerol, and a combination thereof.

Embodiment 194. The pharmaceutical composition of any one of embodiments 171 to 193, wherein the pharmaceutical composition is formulated for oral administration.

Embodiment 195. The pharmaceutical composition of embodiment 194, wherein the pharmaceutical composition comprises a capsule for encapsulating the preparation of fecal bacteria.

Embodiment 196. The pharmaceutical composition of embodiment 195, wherein the capsule comprises a delayed-release coating.

Embodiment 197. A method comprising extracting fecal bacteria from a stool of a healthy human donor to produce a preparation of uncultured fecal bacteria, wherein the healthy human donor is pre-selected for a fecal metabolite at or above a threshold level in one or more, two or more, three or more, four or more, five or more, six or more, seven or more, or ten or more stool samples from the donor.

Embodiment 198. A method comprising extracting fecal bacteria from a stool of a healthy human donor to produce a preparation of uncultured fecal bacteria, wherein one or more, two or more, three or more, four or more, five or more, six or more, seven or more, or ten or more stool samples of the healthy human donor comprise a fecal metabolite at or above a threshold level.

Embodiment 199. A method comprising extracting fecal bacteria from a stool of a healthy human donor to produce a preparation of uncultured fecal bacteria, wherein one or more, two or more, three or more, four or more, five or more, six or more, seven or more, or ten or more stool samples of the healthy human donor are capable of producing a fecal metabolite at or above a threshold level.

Embodiment 200. The method of any one of embodiments 197 to 199, wherein the fecal metabolite is a short-chain fatty acid (SCFA).

Embodiment 201. The method of any one of embodiments 197 to 200, wherein the method comprises determining the level of the metabolite in a raw stool sample of the donor.

Embodiment 202. The method of any one of embodiments 197 to 200, wherein the method comprises determining the level of the metabolite produced by the fecal bacterial cells in a functional assay.

Embodiment 203. The method of embodiment 202, wherein the functional assay is an ex vivo assay.

Embodiment 204. The method of any one of embodiments 197 to 203, further comprising formulating the preparation of uncultured fecal bacteria as a pharmaceutical composition.

Embodiment 205. A method comprising administering a pharmaceutical composition to a subject in need thereof, wherein the pharmaceutical composition comprises a preparation of uncultured fecal bacteria from a stool of a healthy human donor, wherein the subject is pre-selected for at least one SCFA at or below a threshold level in one or more, two or more, three or more, four or more, five or more, six or more, seven or more, or ten or more stool samples from the subject.

Embodiment 206. A method comprising administering a pharmaceutical composition to a subject in need thereof, wherein the pharmaceutical composition comprises a preparation of uncultured fecal bacteria from a stool of a healthy human donor, wherein one or more, two or more, three or more, four or more, five or more, six or more, seven or more, or ten or more stool samples of the subject comprise at least one SCFA at or below a threshold level.

Embodiment 207. A method comprising administering a pharmaceutical composition to a subject in need thereof, wherein the pharmaceutical composition comprises a preparation of uncultured fecal bacteria from a stool of a healthy human donor, wherein one or more, two or more, three or more, four or more, five or more, six or more, seven or more, or ten or more stool samples of the subject are capable of producing at least one SCFA at or below a threshold level.

Embodiment 208. A method comprising administering a pharmaceutical composition to a subject in need thereof, wherein the pharmaceutical composition comprises a preparation of uncultured fecal bacteria from a stool of a healthy human donor, wherein the subject is pre-selected for at least one SCFA at or above a threshold level in one or more, two or more, three or more, four or more, five or more, six or more, seven or more, or ten or more stool samples from the subject.

Embodiment 209. A method comprising administering a pharmaceutical composition to a subject in need thereof, wherein the pharmaceutical composition comprises a preparation of uncultured fecal bacteria from a stool of a healthy human donor, wherein one or more, two or more, three or more, four or more, five or more, six or more, seven or more, or ten or more stool samples of the subject comprise at least one SCFA at or above a threshold level.

Embodiment 210. A method comprising administering a pharmaceutical composition to a subject in need thereof, wherein the pharmaceutical composition comprises a preparation of uncultured fecal bacteria from a stool of a healthy human donor, wherein one or more, two or more, three or more, four or more, five or more, six or more, seven or more, or ten or more stool samples of the subject are capable of producing at least one SCFA at or above a threshold level.

Embodiment 211. The method of any one of embodiments 205 to 210, wherein the at least one SCFA is selected from the group consisting of butyrate, acetate, caproate, heptanoate, isobutyrate, isocaproate, isovalerate, propionate, valerate, and a combination thereof.

Embodiment 212. The method of any one of embodiments 205 to 211, wherein the level of the at least one SCFA is determined from a raw stool of the subject.

Embodiment 213. The method of any one of embodiments 205 to 210, wherein the at least one SCFA is butyrate.

Embodiment 214. The method of embodiment 213, wherein the threshold level of the butyrate is 2 nM per gram raw stool.

Embodiment 215. The method of embodiment 213, wherein the threshold level of the butyrate is 5 nM per gram raw stool.

Embodiment 216. The method of embodiment 213, wherein the threshold level of the butyrate is 10 nM per gram raw stool.

Embodiment 217. The method of any one of embodiments 205 to 210, wherein the at least one SCFA is acetate.

Embodiment 218. The method of embodiment 217, wherein the threshold level of the acetate is 10 nM per gram raw stool.

Embodiment 219. The method of embodiment 217, wherein the threshold level of the acetate is 25 nM per gram raw stool.

Embodiment 220. The method of embodiment 217, wherein the threshold level of the acetate is 40 nM per gram raw stool.

Embodiment 221. The method of any one of embodiments 205 to 210, wherein the at least one SCFA is propionate.

Embodiment 222. The method of embodiment 221, wherein the threshold level of the propionate is 2 nM per gram raw stool.

Embodiment 223. The method of embodiment 221, wherein the threshold level of the propionate is 8 nM per gram raw stool.

Embodiment 224. The method of embodiment 221, wherein the threshold level of the propionate is 12 nM per gram raw stool.

Embodiment 225. The method of any one of embodiments 205 to 224, wherein administering the pharmaceutical composition increases the level of the at least one SCFA in the intestine of the subject above the threshold level.

Embodiment 226. The method of embodiment 225, wherein administering the pharmaceutical composition increases the level of the at least one SCFA produced by intestinal bacteria of the subject by at least 5%.

Embodiment 227. The method of embodiment 225, wherein administering the pharmaceutical composition increases the level of the at least one SCFA produced by intestinal bacteria of the subject by at least 10%.

Embodiment 228. The method of embodiment 225, wherein administering the pharmaceutical composition increases the level of the at least one SCFA produced by intestinal bacteria of the subject by at least 15%.

Embodiment 229. The method of embodiment 225, wherein administering the pharmaceutical composition increases the level of the at least one SCFA produced by intestinal bacteria of the subject by at least 20%.

Embodiment 230. The method of any one of embodiments 205 to 224, wherein administering the pharmaceutical composition decreases the level of the at least one SCFA in the intestine of the subject above the threshold level.

Embodiment 231. The method of embodiment 230, wherein administering the pharmaceutical composition decreases the level of the at least one SCFA produced by intestinal bacteria of the subject by at least 5%.

Embodiment 232. The method of embodiment 230, wherein administering the pharmaceutical composition decreases the level of the at least one SCFA produced by intestinal bacteria of the subject by at least 10%.

Embodiment 233. The method of embodiment 230, wherein administering the pharmaceutical composition decreases the level of the at least one SCFA produced by intestinal bacteria of the subject by at least 15%.

Embodiment 234. The method of embodiment 230, wherein administering the pharmaceutical composition decreases the level of the at least one SCFA produced by intestinal bacteria of the subject by at least 20%.

Embodiment 235. The method of any one of embodiments 205 to 207, wherein the healthy human donor is pre-selected for at least one SCFA at or above a threshold level in one or more, two or more, three or more, four or more, five or more, six or more, seven or more, or ten or more stool samples from the donor.

Embodiment 236. The method of any one of embodiments 205 to 207, wherein one or more, two or more, three or more, four or more, five or more, six or more, seven or more, or ten or more stool samples of the subject comprise at least one SCFA at or above a threshold level.

Embodiment 237. The method of any one of embodiments 205 to 207, wherein one or more, two or more, three or more, four or more, five or more, six or more, seven or more, or ten or more stool samples of the healthy human donor are capable of producing at least one SCFA at or above a threshold level.

Embodiment 238. The method of any one of embodiments 208 to 210, wherein the healthy human donor is pre-selected for at least one SCFA at or below a threshold level in one or more, two or more, three or more, four or more, five or more, six or more, seven or more, or ten or more stool samples from the donor.

Embodiment 239. The method of any one of embodiments 208 to 210, wherein one or more, two or more, three or more, four or more, five or more, six or more, seven or more, or ten or more stool samples of the subject comprise at least one SCFA at or below a threshold level.

Embodiment 240. The method of any one of embodiments 208 to 210, wherein one or more, two or more, three or more, four or more, five or more, six or more, seven or more, or ten or more stool samples of the healthy human donor are capable of producing at least one SCFA at or below a threshold level.

Embodiment 241. A pharmaceutical composition comprising a preparation of fecal bacteria from a stool of a healthy human donor, wherein the fecal bacteria are selected to include at least one bile acid transforming bacterial strain, wherein the fecal bacteria comprises uncultured bacteria, and wherein the preparation of fecal bacteria comprises lyophilized bacteria.

Embodiment 242. The pharmaceutical composition of embodiment 241, wherein the bile acid transforming bacterial strain is capable of transforming at least one primary bile acid selected from the group consisting of cholic acid (CA), chenodeoxycholic acid (CDCA), glycocholic acid (GCA), taurocholic acid (TCA), glycochenodeoxycholic acid (GCDCA), and taurochenodeoxycholic acid (TCDCA).

Embodiment 243. The pharmaceutical composition of any one of embodiment 241 or embodiment 242, wherein the fecal bacteria are selected to produce at least one secondary bile acid at or above a threshold level.

Embodiment 244. The pharmaceutical composition of embodiment 243, wherein the secondary bile acid is selected from the group consisting of deoxycholic acid (DCA), glycodeoxycholic acid (GDCA), taurodeoxycholic acid (TDCA), glycolithocholic acid (GLCA), taurolithocholic acid (TLCA), lithocholic acid (LCA), ursodeoxycholic acid (UDCA) glycoursodeoxycholic acid (GCDCA), and tauroursodeoxycholic acid (TUDCA).

Embodiment 245. The pharmaceutical composition of embodiment 243, wherein the fecal bacteria are selected using a functional assay for determining the level of the at least one bile acid transforming bacterial strain.

Embodiment 246. The pharmaceutical composition of embodiment 245, wherein the functional assay comprises an ex vivo assay.

Embodiment 247. The pharmaceutical composition of embodiment 243, wherein the threshold level of at least one secondary bile acid is 100 μM.

Embodiment 248. The pharmaceutical composition of embodiment 241, wherein the fecal bacteria are selected on the basis of the relative abundance of two or more bile acid transforming bacterial strains in the stool of the donor.

Embodiment 249. The pharmaceutical composition of embodiment 241, wherein the at least one bile acid transforming bacterial strain is a member of a taxonomic group selected from the group consisting of Firmicutes, Bacteroidetes, Eubacterium sp., Clostridium sp., Faecalibacterium sp., Roseburia sp., Butyrivibrio sp., Anaerostipes sp., Coprococcus sp., Subdoligranulum sp., Anaerotruncus sp., Ruminococcus sp., Eubacterium rectale, Roseburia intestinalis, Roseburia faecis, Roseburia hominis, Roseburia inulinivorans, Roseburia cecicola, Butyrivibrio fibrisolvens, Eubacterium ramulus, Eubacterium hallii, Eubacterium ruminantium, Eubacterium cylindroides, Eubacterium oxidoreducens, Coprococcus catus, Coprococcus eutactus, Coprococcus comes, Faecalibacterium prausnitzii, Subdoligranulum variabile, Anaerotruncus colihominis, Clostridium nexile, Clostridium hathewayi, Clostridium indolis, Clostridium leptum, Ruminococcus gnavus, Ruminococcus obeum, and Anaerostipes caccae.

Embodiment 250. The pharmaceutical composition of any one of embodiments 241 to 249, wherein the composition further comprises an SCFA.

Embodiment 251. The pharmaceutical composition of any one of embodiments 241 to 250, wherein the composition further comprises a prebiotic.

Embodiment 252. The pharmaceutical composition of embodiment 251, wherein the prebiotic is selected from the group consisting of inulin, fructooligosaccharide, sunfiber, white navy bean powder, and a combination thereof.

Embodiment 253. The pharmaceutical composition of any one of embodiments 241 to 252, wherein the preparation of fecal bacteria further comprises a cryoprotectant selected from the group consisting of polyethylene glycol, skim milk, erythritol, arabitol, sorbitol, glucose, fructose, alanine, glycine, proline, sucrose, lactose, ribose, trehalose, dimethyl sulfoxide (DMSO), glycerol, and a combination thereof.

Embodiment 254. The pharmaceutical composition of any one of embodiments 241 to 253, wherein the pharmaceutical composition is formulated for oral administration.

Embodiment 255. The pharmaceutical composition of embodiment 241, wherein the pharmaceutical composition comprises a capsule for encapsulating the preparation of fecal bacteria.

Embodiment 256. The pharmaceutical composition of embodiment 241, wherein the capsule comprises a delayed-release coating.

Embodiment 257. The pharmaceutical composition of embodiment 241, wherein the fecal bacteria produce the at least one secondary bile acid in an intestine of a subject administered the composition.

Embodiment 258. A method of selecting a stool of a donor for producing a preparation of uncultured fecal bacteria comprising determining the relative abundance of secondary bile acid in a first stool of an individual is at or above a threshold level, determining that a relative abundance of the secondary bile acid in a second stool of a donor is at or above a threshold level, and selecting the individual as the stool donor based on determining that the relative abundance of the secondary bile acid in the first and second stools is at or above the threshold level.

Embodiment 259. A method of selecting a stool of a donor for producing a preparation of uncultured fecal bacteria comprising determining the relative abundance of secondary bile acid in a stool of an individual and selecting the individual as a stool donor if the relative abundance of the secondary bile acid is at or above a threshold level.

Embodiment 260. A method of selecting a stool of a donor for producing a preparation of fecal bacteria comprising determining the relative abundance of bacteria capable of transforming primary bile acids to secondary bile acids and selecting the stool if the relative abundance of the secondary bile acids is at or above the threshold level.

Embodiment 261. A method of selecting a desired donor by screening potential donors' fecal microbial gene content associated with bile acid transforming strains.

Embodiment 262. A method of treating an irritable bowel disease (IBD) patient in need thereof comprising administering a preparation of fecal bacteria replete with bile acid transforming strains, wherein the patient in need thereof has a deficient bile acid metabolism.

Embodiment 263. A method of treating an irritable bowel disease (IBD) patient in need thereof comprising administering a preparation of fecal bacteria with a relative abundance of secondary bile acids, wherein the patient in need thereof has a deficient bile acid metabolism and the relative abundance of secondary bile acid is at or above a threshold level.

Embodiment 264. A method of treating an irritable bowel disease (IBD) in a patient in need thereof having a deficient bile acid metabolism comprising administering to the patient a preparation of fecal bacteria derived from a donor having a quantity of secondary bile acids above a threshold and bile acid transforming strains.

Embodiment 265. The method of any one of embodiments 262, 263, or 264, wherein the IBD is selected from the group consisting of Crohn's disease, ulcerative colitis, pouchitis, and a combination thereof.

Embodiment 266. The method of any one of embodiments 258, 259, 260, 263, or 264 wherein the threshold level of secondary bile acid is at least 100 μM.

Embodiment 267. A method of treating Crohn's disease or ulcerative colitis in a patient with deficient bile acid metabolism comprising treating the patient with donor materials replete with (i) a quantity of secondary bile acids and (ii) a quantity of bile acid transforming strains, wherein the quantity is at or above a predetermined threshold.

Embodiment 268. The method of embodiment 267, wherein the predetermined threshold of the quantity of secondary bile acids is 100 μM.

Embodiment 269. The method of embodiment 248, wherein the fecal bacteria are selected on the basis of the relative abundance of three or more bile acid transforming bacterial strains in the stool of the donor.

Embodiment 270. A method of treating a Crohn's disease patient with deficient bile acid metabolism by increasing bile acid transforming strains in the patient comprising administering to the patient a preparation of fecal bacteria derived from a donor having a quantity of bile acid transforming strains at or above a threshold level, wherein the patient's bile acid metabolism is restored.

Embodiment 271. A method of treating an ulcerative colitis patient with deficient bile acid metabolism by increasing bile acid transforming strains in the patient comprising administering to the patient a preparation of fecal bacteria derived from a donor having a quantity of bile acid transforming strains at or above a threshold level, wherein the patient's bile acid metabolism is restored.

Embodiment 272. A method of delivering a bile acid transforming bacteria to an intestine of a subject in need thereof, the method comprising administering to the subject a pharmaceutical composition comprising a preparation of uncultured fecal bacteria derived from a stool of a healthy human donor, wherein: the uncultured fecal bacteria are selected to transform primary bile acid to secondary bile acid at or above a threshold level, fecal bacteria in a stool of the healthy human donor produce the secondary bile acid at or above a threshold level, or the healthy human donor produces a stool comprising the secondary bile acid at or above a threshold level.

Embodiment 273. The method of embodiment 272, further comprising administering a prebiotic to the subject.

Embodiment 274. The method of embodiment 272, wherein the prebiotic is selected from the group consisting of an amino acid (e.g., valine, leucine, isoleucine), lactic acid, ammonium nitrate, amylose, barley mulch, biotin, carbonate, cellulose, chitin, choline, fructooligosaccharides (FOSs), fructose, glucose, glycerol, heteropolysaccharide, histidine, homopolysaccharide, hydroxyapatite, inulin, isomaltulose, lactose, lactulose, maltodextrins, maltose, nitrogen, oligodextrose, oligofructose, oligofructose-enriched inulin, an oligosaccharide (e.g. comprising a galactooligosaccharide (GOS), trans-galactooligosaccharide, fructooligosaccharide (FOS), xylooligosaccharides (XOS), mannooligosaccharide, or chitooligosaccharide), pectin, phosphate salts, phosphorus, polydextroses, polyols, potash, potassium, sodium nitrate, starch, sucrose, sulfur, sun fiber, tagatose, thiamine, trehalose, vitamins, a water-soluble carbohydrate, a fermentable polysaccharide, a dietary fiber, resistant starch, barley, white navy bean powder, and a combination thereof.

Embodiment 275. The method of any one of embodiments 272 to 274, further comprising pretreating the subject with an antibiotic before administering the pharmaceutical composition to the subject.

Embodiment 276. The method of any one of embodiments 272 to 275, the secondary bile acid is selected from the group consisting of deoxycholic acid (DCA), glycodeoxycholic acid (GDCA), taurodeoxycholic acid (TDCA), glycolithocholic acid (GLCA), taurolithocholic acid (TLCA), lithocholic acid (LCA), ursodeoxycholic acid (UDCA) glycoursodeoxycholic acid (GUDCA), and tauroursodeoxycholic acid (TUDCA).

Embodiment 277. The method of any one of embodiments 272 to 276, wherein the fecal bacteria are selected by determining a level of the at least one secondary bile acid in a stool of the donor.

Embodiment 278. The method of any one of embodiments 272 to 277, further comprising determining an amount of the secondary bile acid produced by fecal bacteria of the subject to be below a threshold level prior to administering the pharmaceutical composition to the subject.

Embodiment 279. The method of any one of embodiments 272 to 278, wherein administering the pharmaceutical composition treats or prevents a disorder in the subject, wherein the disorder is selected from a C. difficile infection, inflammatory bowel disease, irritable bowel syndrome, and a combination thereof.

Embodiment 280. A method comprising determining a level of at least one secondary bile acid produced by fecal bacteria of a subject comprising at least one bile acid transforming bacterial strain; and administering to the subject a pharmaceutical composition based on determining the level of the at least one secondary bile acid to be below a threshold level, wherein the pharmaceutical composition comprises a preparation of uncultured fecal bacteria of stool of a healthy human donor.

Embodiment 281. The method of embodiment 280, wherein the at least one secondary bile acid is selected from the group consisting of deoxycholic acid (DCA), glycodeoxycholic acid (GDCA), taurodeoxycholic acid (TDCA), glycolithocholic acid (GLCA), taurolithocholic acid (TLCA), lithocholic acid (LCA), ursodeoxycholic acid (UDCA) glycoursodeoxycholic acid (GUDCA), and tauroursodeoxycholic acid (TUDCA).

Embodiment 282. The method of embodiment 280 or embodiment 281, wherein the level of the at least one secondary bile acid is determined from raw stool of the subject.

Embodiment 283. The method of embodiment 281, wherein the at least one secondary bile acid is deoxycholic acid (DCA).

Embodiment 284. The method of embodiment 281, wherein the at least one secondary bile acid is glycodeoxycholic acid (GDCA).

Embodiment 285. The method of embodiment 281, wherein the at least one secondary bile acid is taurodeoxycholic acid (TDCA).

Embodiment 286. The method of embodiment 281, wherein the at least one secondary bile acid is glycolithocholic acid (GLCA).

Embodiment 287. The method of embodiment 281, wherein the at least one secondary bile acid is taurolithocholic acid (TLCA).

Embodiment 288. The method of embodiment 281, wherein the at least one secondary bile acid is lithocholic acid (LCA).

Embodiment 289. The method of embodiment 281, wherein the at least one secondary bile acid is ursodeoxycholic acid (UDCA).

Embodiment 290. The method of embodiment 281, wherein the at least one secondary bile acid is glycoursodeoxycholic acid (GUDCA).

Embodiment 291. The method of embodiment 281, wherein the at least one secondary bile acid is tauroursodeoxycholic acid (TUDCA).

Embodiment 292. The method of embodiment 280, wherein administering the pharmaceutical composition increases the level of the at least one secondary bile acid in the intestine of the subject above the threshold level.

Embodiment 293. The method of embodiment 280, wherein administering the pharmaceutical composition increases the level of the at least one secondary bile acid produced by intestinal bacteria of the subject by at least 5%.

Embodiment 294. The method of embodiment 280, wherein administering the pharmaceutical composition increases the level of the at least one secondary bile acid produced by intestinal bacteria of the subject by at least 10%.

Embodiment 295. The method of embodiment 280, wherein administering the pharmaceutical composition increases the level of the at least one secondary bile acid produced by intestinal bacteria of the subject by at least 15%.

Embodiment 296. The method of embodiment 280, wherein administering the pharmaceutical composition increases the level of the at least one secondary bile acid produced by intestinal bacteria of the subject by at least 20%.

Embodiment 297. The method of embodiment 280, wherein the at least one bile acid transforming bacterial strain is a member of a taxonomic group selected from the group consisting of Firmicutes, Bacteroidetes, Eubacterium sp., Clostridium sp., Faecalibacterium sp., Roseburia sp., Butyrivibrio sp., Anaerostipes sp., Coprococcus sp., Subdoligranulum sp., Anaerotruncus sp., Ruminococcus sp., Eubacterium rectale, Roseburia intestinalis, Roseburia faecis, Roseburia hominis, Roseburia inulinivorans, Roseburia cecicola, Butyrivibrio fibrisolvens, Eubacterium ramulus, Eubacterium hallii, Eubacterium ruminantium, Eubacterium cylindroides, Eubacterium oxidoreducens, Coprococcus catus, Coprococcus eutactus, Coprococcus comes, Faecalibacterium prausnitzii, Subdoligranulum variabile, Anaerotruncus colihominis, Clostridium nexile, Clostridium hathewayi, Clostridium indolis, Clostridium leptum, Ruminococcus gnavus, Ruminococcus obeum, and Anaerostipes caccae.

Embodiment 298. A method comprising: determining a level of a secondary bile acid transformed by fecal bacterial cells to be at or above a threshold level, wherein the fecal bacterial cells are from a stool of a healthy human donor; and extracting fecal bacteria from a stool of the donor based on determining the level of the secondary bile acid to be at or above the threshold level to produce a preparation of uncultured fecal bacteria.

Embodiment 299. The method of embodiment 298, wherein the fecal bacterial cells and the fecal bacteria are from the same stool of the donor.

Embodiment 300. The method of embodiment 298, wherein the fecal bacterial cells and the fecal bacteria are from different stools of the donor.

Embodiment 301. The method of embodiment 298, wherein determining the level of the secondary bile acid comprises determining a level of the secondary bile acid in raw stool of the donor.

Embodiment 302. The method of embodiment 298, wherein determining the level of the secondary bile acid comprises determining a level of the secondary bile acid produced by the fecal bacterial cells in a functional assay.

Embodiment 303. The method of embodiment 302, wherein the functional assay is an ex vivo assay.

Embodiment 304. The method of any one of embodiments 298 to 303, further comprising formulating the preparation of uncultured fecal bacteria as a pharmaceutical composition.

305. The method of embodiment 298, wherein the at least one secondary bile acid is selected from the group consisting of deoxycholic acid (DCA), glycodeoxycholic acid (GDCA), taurodeoxycholic acid (TDCA), glycolithocholic acid (GLCA), taurolithocholic acid (TLCA), lithocholic acid (LCA), ursodeoxycholic acid (UDCA) glycoursodeoxycholic acid (GUDCA), and tauroursodeoxycholic acid (TUDCA).

Embodiment 306. A method comprising: determining that a level of secondary bile acids transformed by fecal bacterial cells is at or above a threshold level, wherein the fecal bacterial cells are from a stool of a healthy human donor; selecting fecal bacteria of the donor based on determining that the level of the secondary bile acid is at or above the threshold level, wherein the selected fecal bacteria are uncultured; mixing the selected fecal bacteria with a cryoprotectant to produce a preparation of uncultured fecal bacteria.

Embodiment 307. The method of embodiment 306, wherein the fecal bacteria are selected from the stool of the donor used to determine that the level of the secondary bile acid produced by the fecal bacterial cells is at or above the threshold level.

Embodiment 308. The method of embodiment 306, wherein the fecal bacterial cells are selected from a different stool of the donor than the stool used to determine that the level of the secondary bile acid transformed by the fecal bacterial cells is at or above the threshold level.

Embodiment 309. The method of embodiment 306, wherein determining the level of the secondary bile acid comprises determining a level of the secondary bile acid transformed by the fecal bacterial cells in a functional assay.

Embodiment 310. The method of embodiment 309, wherein the functional assay comprises an ex vivo assay.

Embodiment 311. The method of embodiment 306, wherein determining the level of the secondary bile acid comprises determining a level of the secondary bile acid in raw stool of the donor.

Embodiment 312. The method of embodiment 306, wherein the secondary bile acid is selected from the group consisting of deoxycholic acid (DCA), glycodeoxycholic acid (GDCA), taurodeoxycholic acid (TDCA), glycolithocholic acid (GLCA), taurolithocholic acid (TLCA), lithocholic acid (LCA), ursodeoxycholic acid (UDCA) glycoursodeoxycholic acid (GUDCA), tauroursodeoxycholic acid (TUDCA) and a combination thereof.

Embodiment 313. The method of embodiment 312, wherein the at least one secondary bile acid is deoxycholic acid (DCA) and the threshold level is 100 μM.

Embodiment 314. The method of embodiment 312, wherein the at least one secondary bile acid is glycodeoxycholic acid (GDCA) and the threshold level is 100 μM.

Embodiment 315. The method of embodiment 312, wherein the at least one secondary bile acid is taurodeoxycholic acid (TDCA) and the threshold level is 100 μM.

Embodiment 316. The method of embodiment 312, wherein the at least one secondary bile acid is glycolithocholic acid (GLCA) and the threshold level is 100 μM.

Embodiment 317. The method of embodiment 312, wherein the at least one secondary bile acid is taurolithocholic acid (TLCA) and the threshold level is 100 μM.

Embodiment 318. The method of embodiment 312, wherein the at least one secondary bile acid is lithocholic acid (LCA) and the threshold level is 300 μM.

Embodiment 319. The method of embodiment 312, wherein the at least one secondary bile acid is ursodeoxycholic acid (UDCA) and the threshold level is 100 μM.

Embodiment 320. The method of embodiment 312, wherein the at least one secondary bile acid is glycoursodeoxycholic acid (GUDCA) and the threshold level is 100 μM.

Embodiment 321. The method of embodiment 312, wherein the at least one secondary bile acid is tauroursodeoxycholic acid (TUDCA) and the threshold level is 100 μM.

Embodiment 322. The method of any one of embodiments 306 to 321, wherein the method further comprises extracting the selected fecal bacteria from a stool of the donor prior to mixing the selected fecal bacteria with the cryoprotectant.

Embodiment 323. The method of embodiment 322, wherein the extracting comprises filtering the selected fecal bacteria.

Embodiment 324. The method of any one of embodiments 306 to 323, wherein the cryoprotectant is selected from the group consisting of polyethylene glycol, skim milk, erythritol, arabitol, sorbitol, glucose, fructose, alanine, glycine, proline, sucrose, lactose, ribose, trehalose, dimethyl sulfoxide (DMSO), glycerol, and a combination thereof.

Embodiment 325. The method of any one of embodiments 306 to 324, wherein the preparation of uncultured fecal bacteria further comprises an antioxidant.

Embodiment 326. The method of any one of embodiments 306 to 325, wherein the method further comprises encapsulating the preparation of uncultured fecal bacteria in a pharmaceutically acceptable carrier.

Embodiment 327. The method of embodiment 326, wherein the pharmaceutically acceptable carrier comprises a tablet, geltab, pill or capsule.

Embodiment 328. The method of embodiment 326, wherein the pharmaceutically acceptable carrier is a capsule, and the capsule is acid-resistant.

Embodiment 329. A method comprising: determining that a relative abundance of one or more bile acid transforming bacterial strains is at or above a threshold level in stool of a healthy human donor; selecting fecal bacteria of the donor based on determining that the relative abundance of the one or more bile acid transforming bacterial strains is at or above the threshold level, wherein the selected fecal bacteria are uncultured; and mixing the selected fecal bacteria with a cryoprotectant to produce a preparation of uncultured fecal bacteria.

Embodiment 330. The method of embodiment 329, wherein the fecal bacteria are selected from the stool of the donor used to determine that the relative abundance of one or more bile acid transforming bacterial strains is at or above the threshold level.

Embodiment 331. The method of embodiment 329, wherein the fecal bacteria are selected from a different stool of the donor than the stool used to determine that the relative abundance of one or more bile acid transforming bacterial strains is at or above the threshold level.

Embodiment 332. The method of any one of embodiments 329 to 331, wherein the one or more SCFA-producing bacterial strains is a member of a taxonomic group selected from the group consisting of Firmicutes, Bacteroidetes, Eubacterium sp., Clostridium sp., Faecalibacterium sp., Roseburia sp., Butyrivibrio sp., Anaerostipes sp., Coprococcus sp., Subdoligranulum sp., Anaerotruncus sp., Ruminococcus sp., Eubacterium rectale, Roseburia intestinalis, Roseburia faecis, Roseburia hominis, Roseburia inulinivorans, Roseburia cecicola, Butyrivibrio fibrisolvens, Eubacterium ramulus, Eubacterium hallii, Eubacterium ruminantium, Eubacterium cylindroides, Eubacterium oxidoreducens, Coprococcus catus, Coprococcus eutactus, Coprococcus comes, Faecalibacterium prausnitzii, Subdoligranulum variabile, Anaerotruncus colihominis, Clostridium nexile, Clostridium hathewayi, Clostridium indolis, Clostridium leptum, Ruminococcus gnavus, Ruminococcus obeum, and Anaerostipes caccae.

Embodiment 333. The method of any one of embodiments 329 to 332, wherein the relative abundance of the one or more bile acid transforming bacterial strains is at least 10%.

Embodiment 334. The method of any one of embodiments 329 to 333, wherein the relative abundance of the one or more bile acid transforming bacterial strains is at least 25%.

Embodiment 335. The method of any one of embodiments 329 to 334, wherein the relative abundance of the one or more bile acid transforming bacterial strains is at least 50%.

Embodiment 336. A pharmaceutical composition comprising a preparation of fecal bacteria from stool of a healthy human donor, wherein the fecal bacteria transform an amount of primary bile acid to secondary bile acid, wherein the secondary bile acid is at or above a threshold level of secondary bile acid when incubated with a substrate in a functional assay, wherein the preparation of fecal bacteria comprises uncultured bacteria, and wherein the threshold level of the secondary bile acid is at least 100 μM.

Embodiment 337. The method of embodiment 336, wherein the at least one secondary bile acid is deoxycholic acid (DCA) and the threshold level is 100 μM.

Embodiment 338. The method of embodiment 336, wherein the at least one secondary bile acid is glycodeoxycholic acid (GDCA) and the threshold level is 50 μM.

Embodiment 339. The method of embodiment 336, wherein the at least one secondary bile acid is taurodeoxycholic acid (TDCA) and the threshold level is 50 μM.

Embodiment 340. The method of embodiment 336, wherein the at least one secondary bile acid is glycolithocholic acid (GLCA) and the threshold level is 50 μM.

Embodiment 341. The method of embodiment 336, wherein the at least one secondary bile acid is taurolithocholic acid (TLCA) and the threshold level is 50 μM.

Embodiment 342. The method of embodiment 336, wherein the at least one secondary bile acid is lithocholic acid (LCA) and the threshold level is 300 μM.

Embodiment 343. The method of embodiment 336, wherein the at least one secondary bile acid is ursodeoxycholic acid (UDCA) and the threshold level is 50 μM.

Embodiment 344. The method of embodiment 336, wherein the at least one secondary bile acid is glycoursodeoxycholic acid (GUDCA) and the threshold level is 50 μM.

Embodiment 345. The method of embodiment 336, wherein the at least one secondary bile acid is tauroursodeoxycholic acid (TUDCA) and the threshold level is 50 μM.

Embodiment 346. A method comprising extracting fecal bacteria from a stool of a healthy human donor to produce a preparation of uncultured fecal bacteria, wherein the healthy human donor is pre-selected for secondary bile acid at or above a threshold level in one or more, two or more, three

The disclosure may be better understood by reference to the following non-limiting Examples, which are provided as exemplary of the disclosure. The following examples are presented in order to more fully illustrate the preferred aspects of the disclosure and should in no way be construed, however, as limiting the broad scope of the disclosure. Therefore, the scope of the appended claims should not be limited to the description of the aspects contained herein.

### EXAMPLES

### Example 1: Quantification of SCFA Using an Ex Vivo Fecal Microbiota Assay

10 ml raw stool is centrifuged in 15 ml conical tubes at 3220×g for 15 minutes. The supernatant is discarded, 10 ml of 0.1M sodium phosphate buffer, pH 7 (SPB) is added to the pellet, and the tube is vortexed until the pellet is homogenized to produce a fecal bacteria suspension. 150 μl of the fecal bacteria is mixed with 150 μl of each SCFA substrate in five different 96-well plates (representing time points 0 h, 6 h, 9 h, 12 h, and 24 h). SCFA substrates are agave inulin (2 g/50 ml SPB in 0.05% L-cysteine), fructooligosaccharide (FOS) (2 g/50 ml SPB in 0.05% L-cysteine), sunfiber partially hydrolyzed guar gum (PHGG) (2 g/50 ml SPB in 0.05% L-cysteine), white navy bean powder (2 g/50 ml SPB in 0.05% L-cysteine), and filtered white navy bean powder (2 g/50 ml SPB, centrifuged at 32.20×g for 5 minutes, heated to 60° C. and sterile filtered, and L-cysteine added to 0.05%). At each timepoint, the respective assay plate is immediately placed on dry ice for 2-5 minutes, and the plate is centrifuged at 3220×g for 15 minutes. 100 μl of supernatant is mixed with 10 μl of 50% sulfuric acid and 500 μl of diethyl ether containing 5 mM 2-methylpentanoic acid in a 2 mL glass vial. Samples are centrifuged at 3,220 g for 10 minutes to obtain a clear phase boundary. SCFA are were measured using a Gas Chromatograph (GC) with a Flame Ionization Detector (FID) using the following parameters:
Injection: A 1 μL sample 1 injected at a sample depth of 8 mm with a fast plunger speed and four sample washes.
Inlet: Split mode at 225° C. with a 20:1 split ratio.
Carrier Gas: Helium
Oven: Isothermal temperature program at 140° C. for 5 min.
Column: Nitroterephthalic-acid modified/polyethylene glycol (PEG) capillary column that is 0.25 mm in diameter, approximately 30 m long with a 0.25 μm film thickness. Column kept at a constant flow of 6.0 mL/min.
Flame Ionization Detector (FID): Temperature set at 225° C. Flow rate for hydrogen is 30.0 mL/min. Flow rate for air is 400.0 mL/min. Makeup flow of helium is 20.0 mL/min.

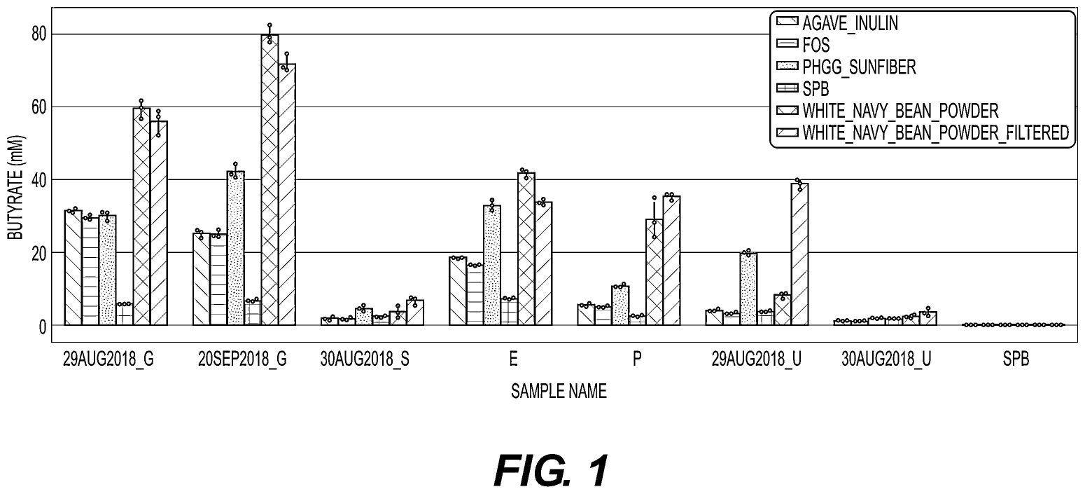

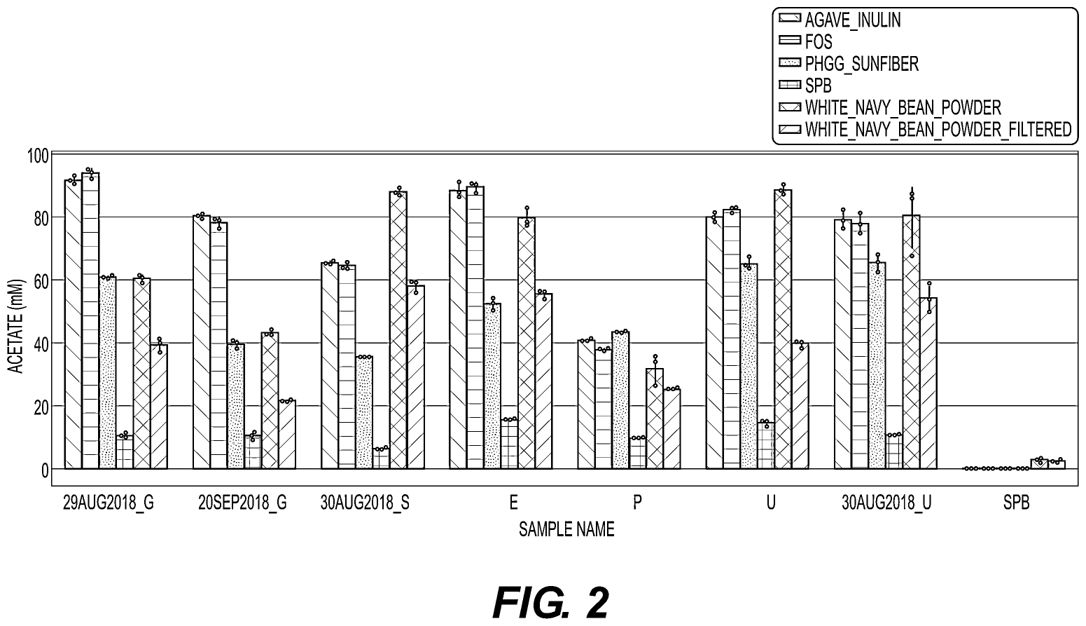

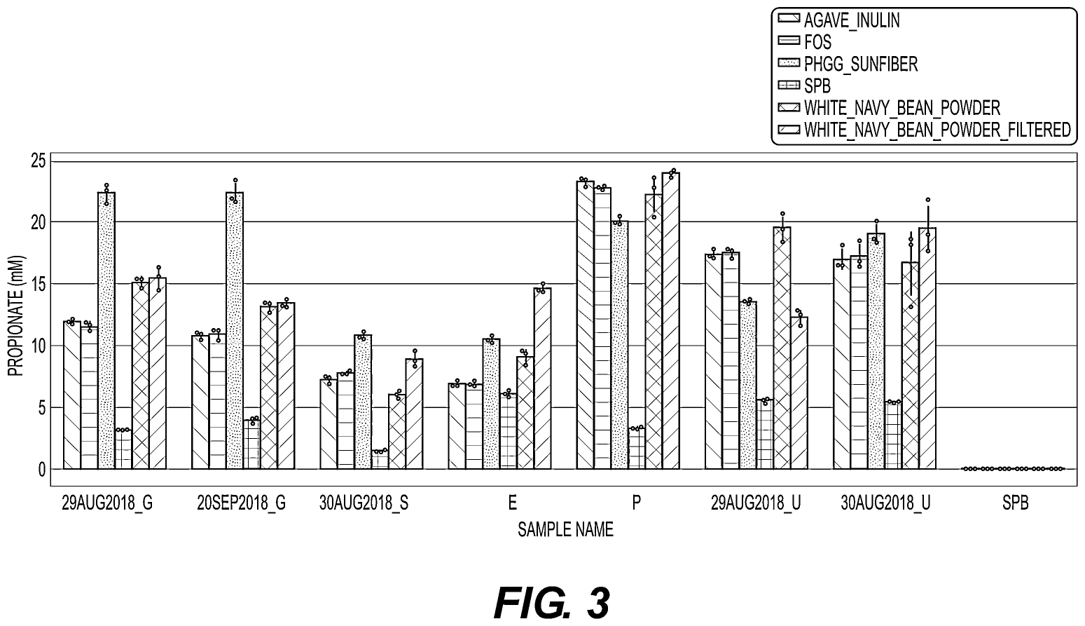

FIG. 1 shows the concentration of butyrate (in mM) produced by the fecal bacteria at the 12 hour timepoint. FIG. 2 shows the concentration of acetate (in mM) produced by the fecal bacteria at the 12 hour timepoint. FIG. 3 shows the concentration of propionate (in mM) produced by the fecal bacteria at the 12 hour timepoint. Each graph shows results from stool of five different donors (1, 16, 76, 81, and 8). For donors 1 and 8, results from two different stool samples are shown. The results of the ex vivo assay show that the level of SCFA production by fecal bacteria is variable across donors. For example, fecal bacteria from donor 1 produce higher levels of butyrate in the ex vivo assay than fecal bacteria from the other donors shown.

### Example 2: Quantification of SCFA Directly from Raw Stool

250 mg of raw stool is mixed with 1.25 ml of HPLC water in a 5 ml conical tube. Samples are centrifuged for 15 minutes at 3200×g. 100 μl of supernatant is mixed with 10 μl of 50% sulfuric acid and 500 μl of diethyl ether containing 5 mM 2-methylpentanoic acid in a 2 mL glass vial. Samples are centrifuged at 3,220 g for 10 minutes to obtain a clear phase boundary. SCFAs are measured using a Gas Chromatograph (GC) with a Flame Ionization Detector (FID) as described in Example 1.

The results of the quantification are shown in Table 2 for the SCFAs acetate (A), butyrate (B), caproate (C), heptanoate (H), isobutyrate (Ib), isocaproate (Ic), isovalerate (Iv), propionate (P), and valerate (V). Similar to the results from ex vivo assay, Table 2 shows that stool SCFA levels vary across donors. For example, stool derived from donor 7 contains higher levels of butyrate than stool of the other donors shown.

TABLE 2

| | Total SCFA (mM) | | | | | | | | | nmol/g Raw Stool | | | | | | | | |
|---|---|---|---|---|---|---|---|---|---|---|---|---|---|---|---|---|---|---|
| Name | A | B | C | H | Ib | Ic | Iv | P | V | A | B | C | H | Ib | Ic | Iv | P | V |
| 30AUG2018_C | 14.8 | 2.75 | 0 | 0 | 0 | 0 | 0 | 2.50 | 0 | 89.1 | 16.5 | 0 | 0 | 0 | 0 | 0 | 15.0 | 0 |
| 30AUG2018_C | 13.9 | 2.46 | 0 | 0 | 0 | 0 | 0 | 2.51 | 0 | 83.6 | 14.8 | 0 | 0 | 0 | 0 | 0 | 15.1 | 0 |
| 30AUG2018_C | 12.5 | 2.66 | 0 | 0 | 0 | 0 | 0 | 2.15 | 0 | 76.0 | 16.2 | 0 | 0 | 0 | 0 | 0 | 13.1 | 0 |
| 30Aug2018_D | 15.8 | 4.90 | 0 | 0 | 0 | 0 | 0 | 7.41 | 0 | 94.1 | 29.1 | 0 | 0 | 0 | 0 | 0 | 44.1 | 0 |
| 30Aug2018_D | 16.9 | 4.74 | 0 | 0 | 0 | 0 | 0 | 6.94 | 0 | 100.5 | 28.3 | 0 | 0 | 0 | 0 | 0 | 41.3 | 0 |
| 30Aug2018_D | 16.1 | 4.93 | 0 | 0 | 0 | 0 | 0 | 7.17 | 0 | 93.5 | 28.6 | 0 | 0 | 0 | 0 | 0 | 41.6 | 0 |
| 30AUG2018_F | 9.6 | 3.29 | 0 | 0 | 0.69 | 0 | 1.15 | 3.70 | 0.83 | 56.7 | 19.5 | 0 | 0 | 4.06 | 0 | 6.81 | 21.9 | 4.92 |
| 30AUG2018_F | 10.7 | 3.63 | 0.76 | 0 | 0.81 | 0 | 1.23 | 3.90 | 0.88 | 64.6 | 22.0 | 4.62 | 0 | 4.89 | 0 | 7.47 | 23.6 | 5.34 |
| 30AUG2018_F | 9.8 | 3.28 | 0 | 0 | 0.65 | 0 | 1.11 | 3.62 | 0.78 | 59.6 | 20.1 | 0 | 0 | 3.98 | 0 | 6.77 | 22.1 | 4.79 |
| 29AUG2018_G | 8.8 | 2.06 | 1.38 | 1.87 | 0.66 | 0.84 | 1.07 | 2.25 | 1.12 | 52.3 | 12.2 | 8.19 | 11.07 | 3.94 | 4.99 | 6.34 | 13.4 | 6.62 |
| 29AUG2018_G | 9.1 | 1.99 | 1.06 | 1.39 | 0.70 | 0 | 0.98 | 2.22 | 0.92 | 51.8 | 11.3 | 6.04 | 7.91 | 3.95 | 0 | 5.56 | 12.6 | 5.22 |
| 29AUG2018_G | 10.4 | 1.96 | 0 | 0 | 0.68 | 0 | 1.07 | 2.30 | 0.77 | 63.8 | 12.0 | 0 | 0 | 4.20 | 0 | 6.58 | 14.1 | 4.70 |
| 20SEP2018_H | 9.8 | 2.07 | 0 | 0 | 0.65 | 0 | 0.82 | 3.65 | 0 | 59.8 | 12.6 | 0 | 0 | 3.95 | 0 | 4.99 | 22.2 | 0 |
| 20SEP2018_H | 11.4 | 2.14 | 0 | 0 | 0.50 | 0 | 0.73 | 3.43 | 0 | 66.5 | 12.5 | 0 | 0 | 2.92 | 0 | 4.29 | 20.0 | 0 |
| 20SEP2018_H | 13.1 | 2.28 | 0 | 0 | 0.70 | 0 | 0.88 | 3.99 | 0.68 | 79.2 | 13.8 | 0 | 0 | 4.22 | 0 | 5.31 | 24.1 | 4.15 |
| 30AUG2018_S | 10.3 | 2.50 | 0 | 0 | 0 | 0 | 0 | 3.70 | 0 | 63.0 | 15.2 | 0 | 0 | 0 | 0 | 0 | 22.5 | 0 |
| 30AUG2018_S | 9.8 | 2.87 | 0 | 0 | 0 | 0 | 0 | 4.23 | 0 | 56.3 | 16.4 | 0 | 0 | 0 | 0 | 0 | 24.2 | 0 |
| 30AUG2018_S | 10.3 | 2.47 | 0 | 0 | 0 | 0 | 0 | 3.71 | 0 | 61.2 | 14.7 | 0 | 0 | 0 | 0 | 0 | 22.1 | 0 |
| 29AUG2018_J | 15.4 | 5.62 | 0.90 | 0 | 0.70 | 0 | 0.91 | 4.72 | 0.98 | 90.6 | 33.1 | 5.30 | 0 | 4.14 | 0 | 5.39 | 27.8 | 5.76 |
| 29AUG2018_J | 15.8 | 6.35 | 1.05 | 0 | 0.63 | 0 | 0.92 | 5.15 | 1.05 | 92.5 | 37.3 | 6.14 | 0 | 3.71 | 0 | 5.37 | 30.2 | 6.15 |
| 29AUG2018_J | 16.7 | 7.14 | 0.95 | 0 | 0.63 | 0 | 0.79 | 5.46 | 0.98 | 99.2 | 42.6 | 5.66 | 0 | 3.76 | 0 | 4.72 | 32.5 | 5.83 |
| 29AUG2018_K | 12.6 | 3.25 | 0 | 0 | 0 | 0 | 0 | 3.34 | 0 | 76.4 | 19.6 | 0 | 0 | 0 | 0 | 0 | 20.2 | 0 |
| 29AUG2018_K | 11.1 | 3.08 | 0 | 0 | 0 | 0 | 0.63 | 3.44 | 0.74 | 68.0 | 18.8 | 0 | 0 | 0 | 0 | 3.83 | 21.0 | 4.52 |
| 29AUG2018_K | 13.8 | 3.21 | 0 | 0 | 0 | 0 | 0 | 3.57 | 0.73 | 83.2 | 19.4 | 0 | 0 | 0 | 0 | 0 | 21.6 | 4.41 |
| 29AUG2018_L | 17.1 | 9.27 | 1.44 | 0 | 0 | 0 | 0 | 5.61 | 1.11 | 100.8 | 54.5 | 8.45 | 0 | 0 | 0 | 0 | 33.0 | 6.52 |
| 29AUG2018_L | 17.0 | 8.52 | 1.24 | 0 | 0 | 0 | 0 | 5.17 | 1.02 | 97.6 | 49.0 | 7.12 | 0 | 0 | 0 | 0 | 29.7 | 5.84 |
| 29AUG2018_L | 15.0 | 7.56 | 1.27 | 0 | 0 | 0 | 0 | 4.82 | 1.02 | 87.7 | 44.3 | 7.46 | 0 | 0 | 0 | 0 | 28.2 | 5.95 |
| 29AUG2018_U | 7.2 | 2.78 | 0 | 0 | 0 | 0 | 0.61 | 3.38 | 0 | 41.8 | 16.2 | 0 | 0 | 0 | 0 | 3.53 | 19.7 | 0 |
| 29AUG2018_U | 10.5 | 2.30 | 0 | 0 | 0 | 0 | 0.55 | 3.53 | 0 | 62.0 | 13.5 | 0 | 0 | 0 | 0 | 3.22 | 20.8 | 0 |
| 29AUG2018_U | 7.8 | 2.77 | 0 | 0 | 0 | 0 | 0.62 | 3.35 | 0 | 43.7 | 15.5 | 0 | 0 | 0 | 0 | 3.46 | 18.8 | 0 |

### Example 3: Production of a Bacterial Mixture Selected to Produce Butyrate

A stool is collected from a screened, healthy human donor and 10 ml of the stool aliquoted for use in determining butyrate production capability of the fecal bacteria in the stool according to the ex vivo assay described in Example 1, using white navy bean powder as the substrate. The remaining portion of the stool sample is diluted with 15% trehalose and 0.05% cysteine in PBS saline, homogenized and filtered. The filtrate is stored at −80° C. If the results of the ex vivo assay show that butyrate is present in the stool sample at a level of greater than 30 mM, then the filtrate is thawed and lyophilized to give rise to a lyophilized preparation of uncultured fecal bacteria. The lyophilized preparation is then encapsulated in an acid-resistant capsule to produce a pharmaceutical composition containing uncultured fecal bacteria selected to produce butyrate.

### Example 4: Treatment of IBD with Uncultured Fecal Bacteria that Produce Butyrate

The butyrate level in stool samples of two stool donors (A and B) is quantified directly from aliquots of the samples. The remaining portions of the stool samples are used to prepare uncultured fecal bacteria preparations that are administered by FMT colonoscopy to patients diagnosed with IBD (ulcerative colitis (UC) and Crohn's Disease (CD)). The post-FMT butyrate level in stool of each patient is then quantified directly from each patient's stool at timepoints of 1 week, 8 weeks and 12 weeks after FMT administration. Butyrate levels for each patient are then averaged across timepoints, and baseline concentrations subtracted.

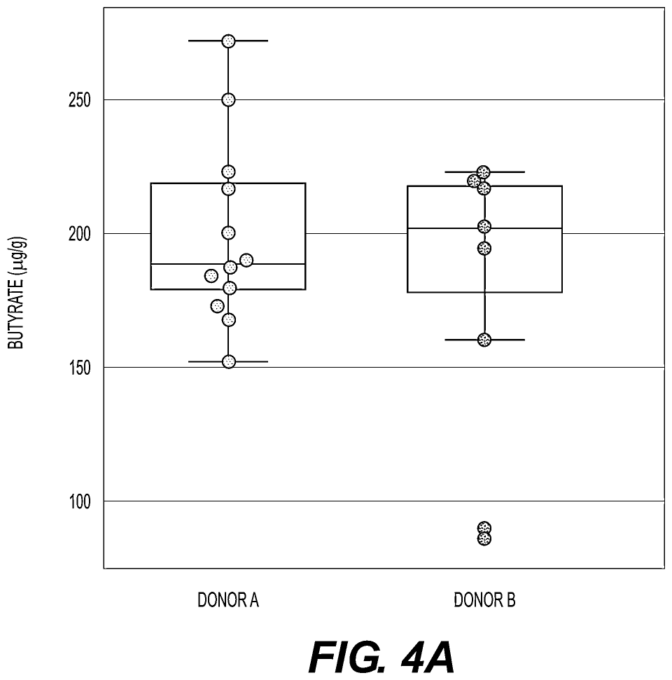

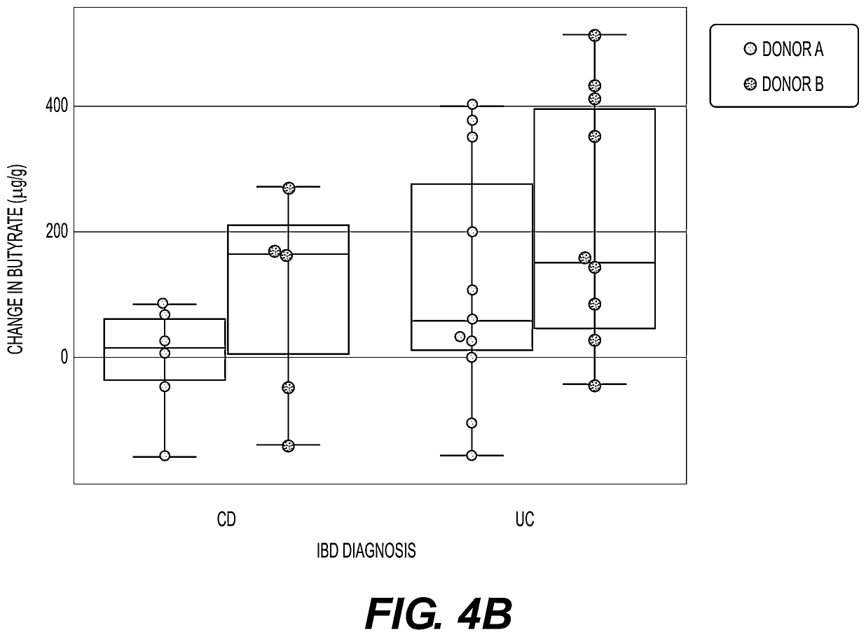

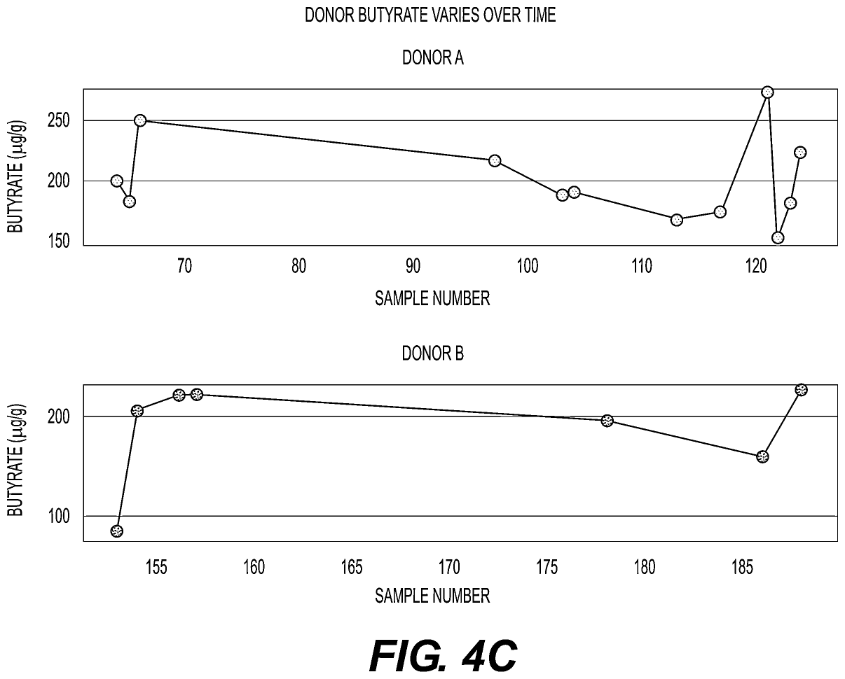

FIG. 4A shows that the quantity of butyrate in donor stool was determined to be approximately 190 μg/g for donor A and 200 μg/g for donor B. The change in butyrate level (in μg/g) in stool of patients after receiving an FMT from each donor is shown in FIG. 4B. For both CD and UC patient diagnoses, the change in patient butyrate induced by FMTs containing fecal bacteria of donor B is greater than the change in patient butyrate induced by FMTs containing fecal bacteria of donor A. These results show that fecal bacteria selected for their ability to produce butyrate in the stool of a donor can induce higher levels of butyrate in patients following FMT administration, compared to fecal bacteria that produce lower levels of butyrate.

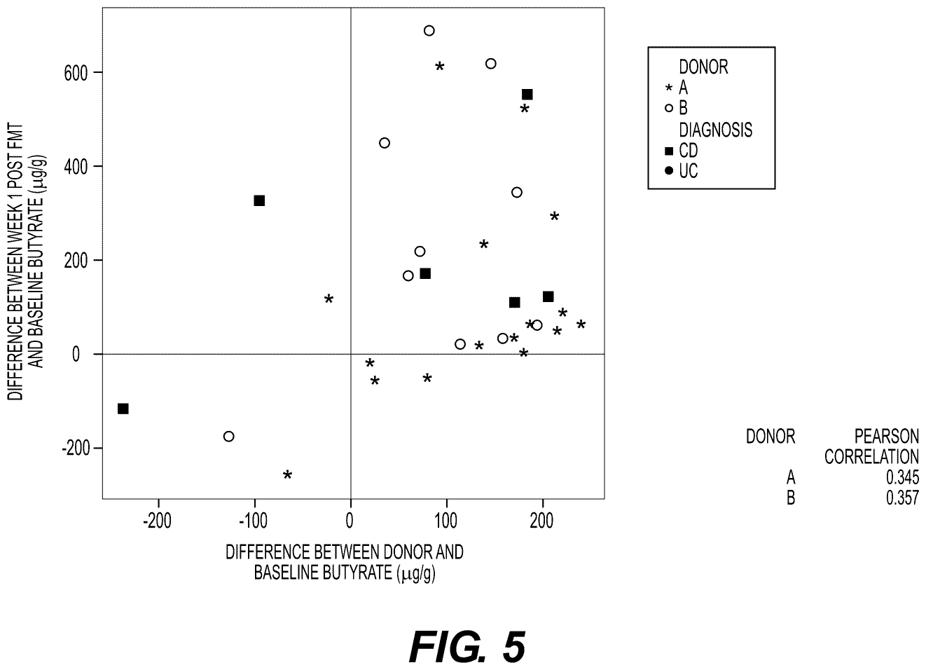

FIG. 4C shows that the quantity of butyrate in donor stool can vary over time. Changes between post-FMT and baseline butyrate in a treated CD or UC patient correlates with the differences between donor butyrate and patient baseline butyrate levels (FIG. 5). Therefore, a donor can be selected based on the difference in butyrate levels between stool of donors and pre-FMT patients, in order to provide for larger increases in butyrate level post-FMT in treated patients.

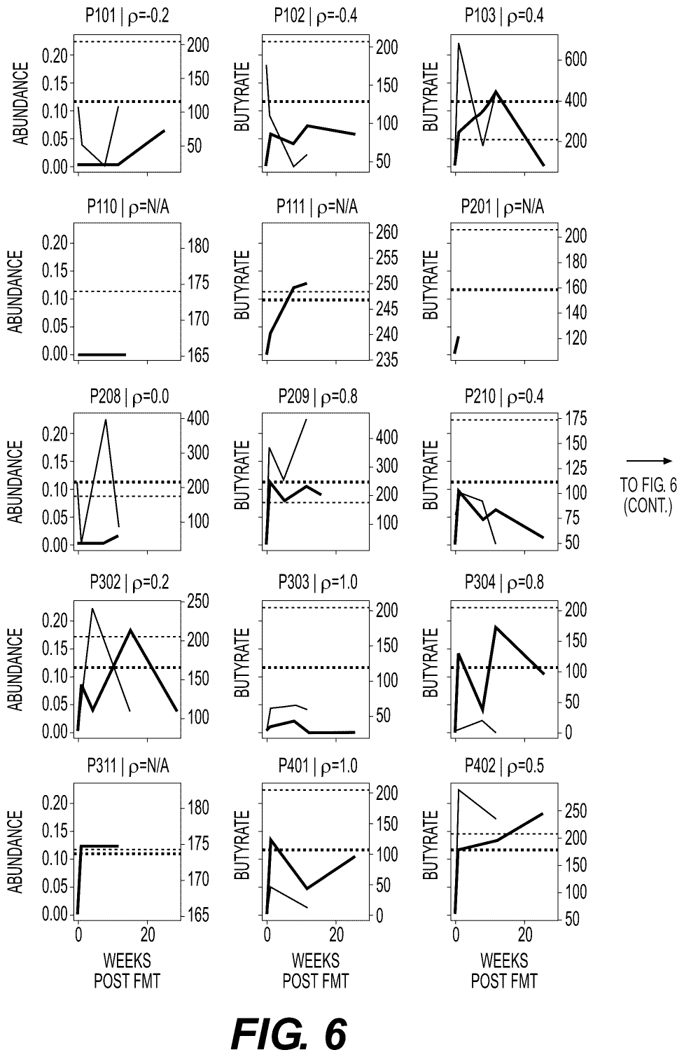

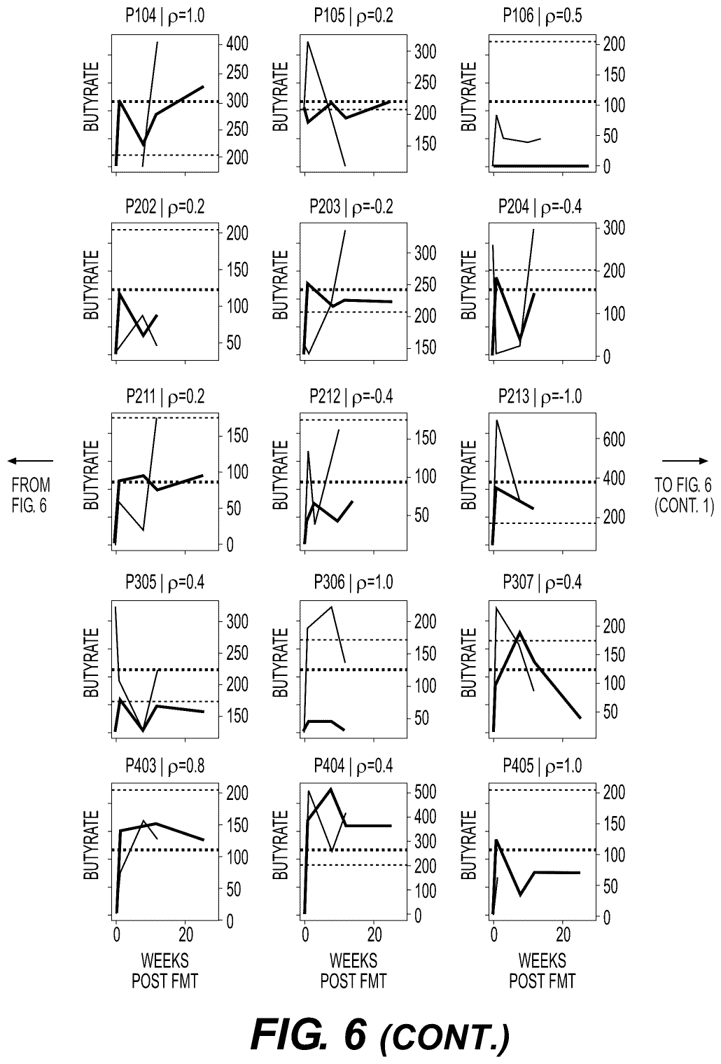

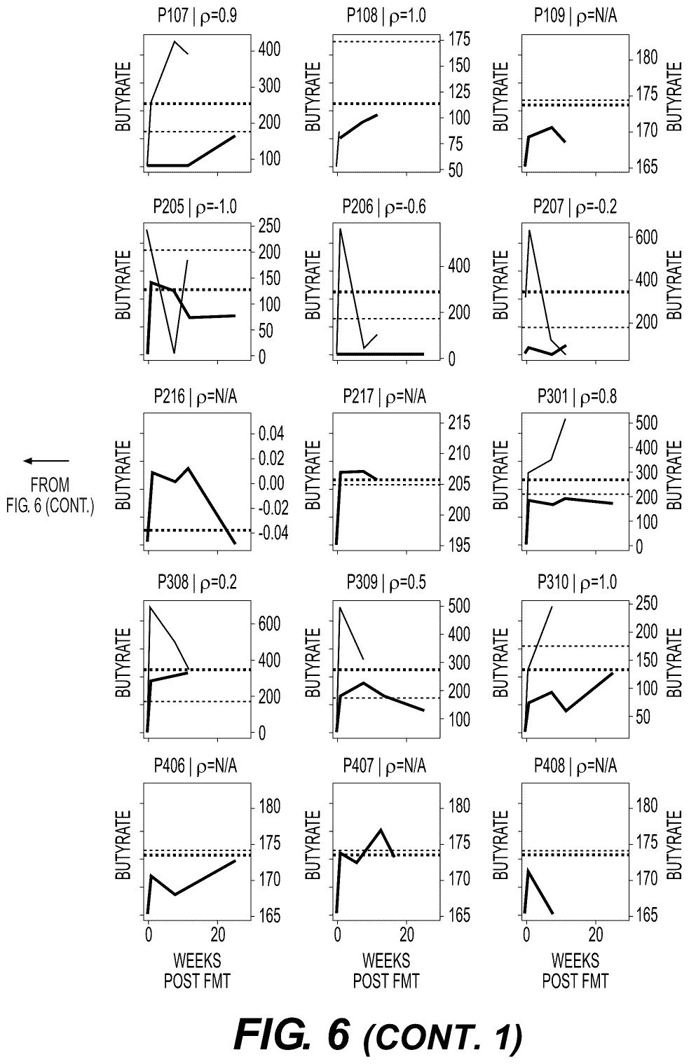

Studies report that Faecalibacterium prausnitzii and Eubacterium rectale/Roseburia spp. are important butyrate producing bacteria. See Louis, P. & Flint, H., FEMS Microbiol Lett. 294: 1-8 (2009) (incorporated herein by reference). FIG. 6 shows that analysis of patient samples following FMT treatment show that Faecalibacterium abundance (black lines) often moves in concert with butyrate (grey lines).

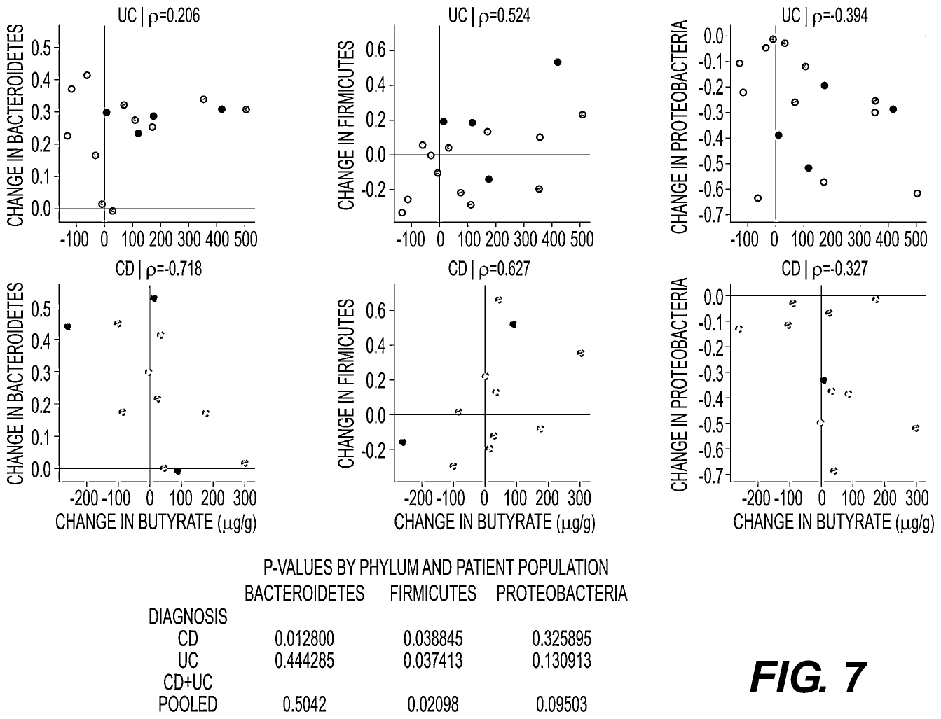

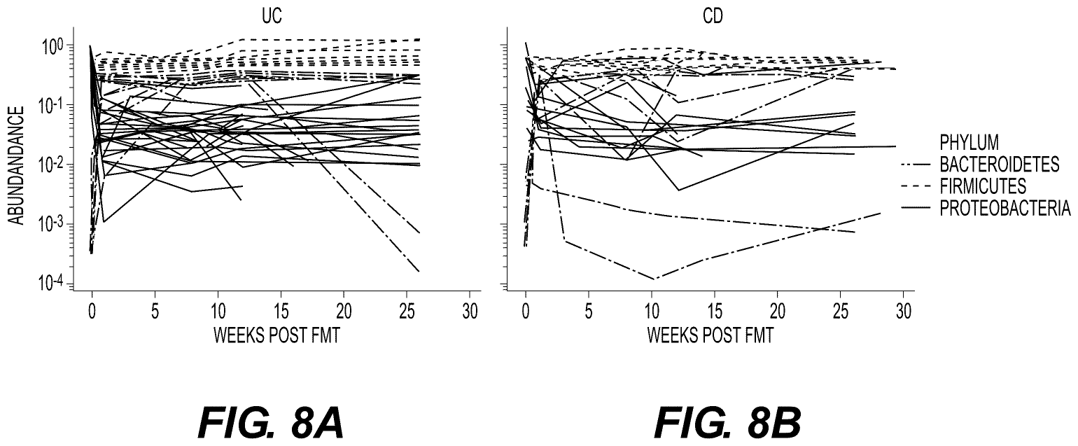

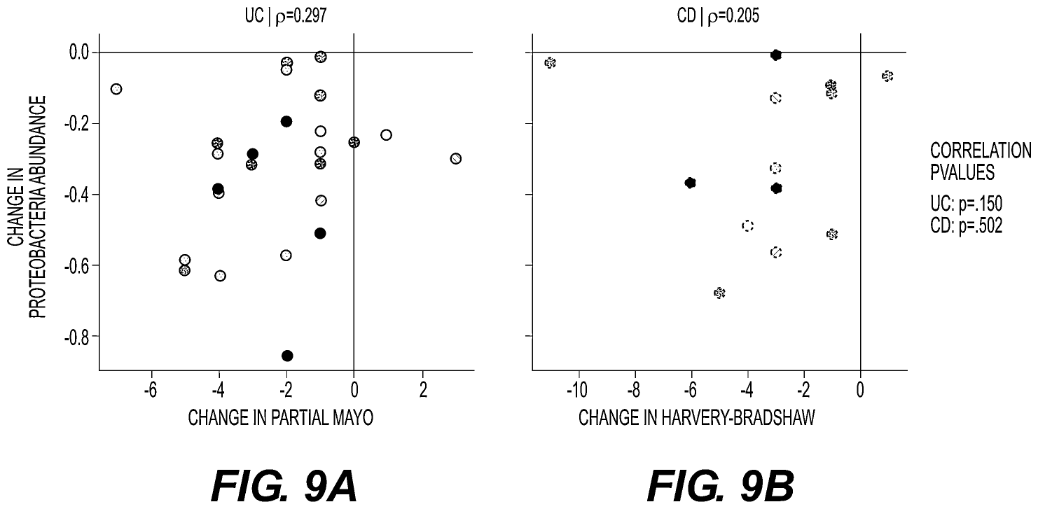

FIG. 7. shows that increases in butyrate significantly correlates with increase in Firmicutes and decrease in Proteobacteria in both Ulcerative Colitis (UC) and Crohn's Disease (CD) patients at 12 weeks post-FMT treatment. Also post-FMT treatment, both Firmicutes and Bacteroidetes increase, while Proteobacteria levels decrease in UC and CD patients (FIG. 8). A review of patient partial Mayo index and Harvery-Bradshaw index show that patient IBD improvement weakly correlates with a decrease in Proteobacteria (FIG. 9; correlation p-values for UC, p=0.150 and CD, p=0.502).

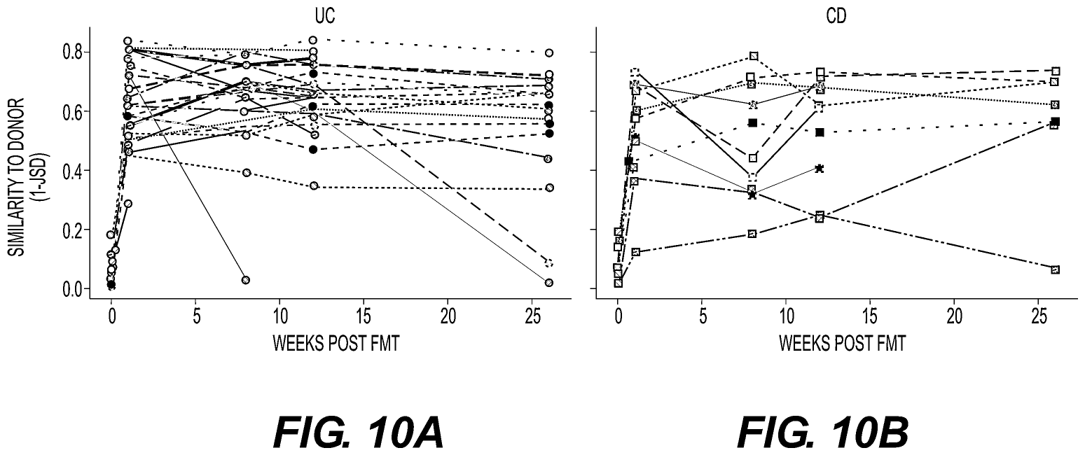

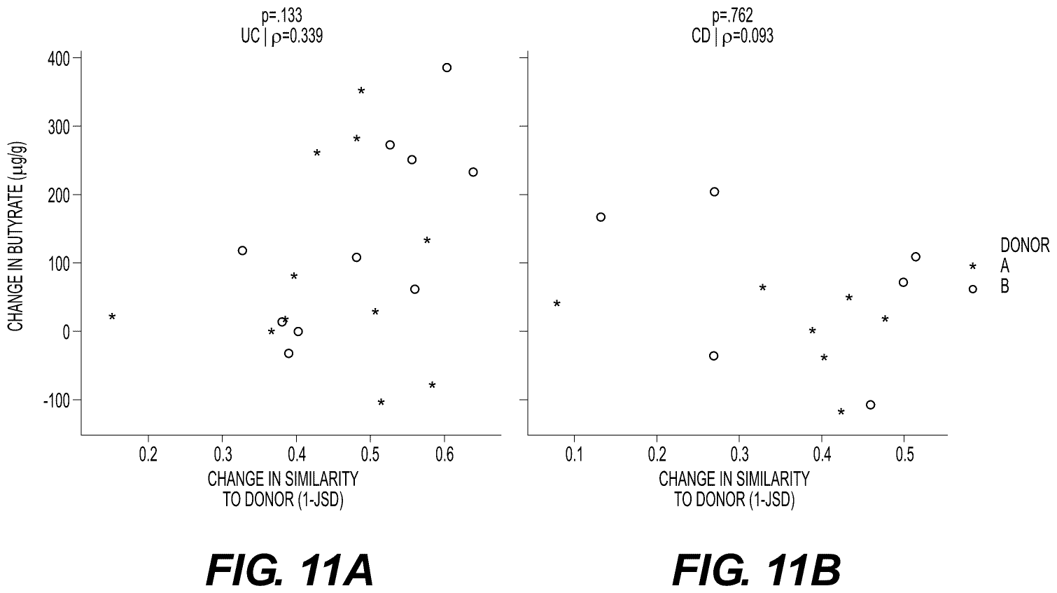

The microbiota bacterial composition of UC and CD patients treated with donor FMT becomes more similar to the microbiota bacterial composition of the donor post-FMT treatment (FIG. 10). FIG. 11 shows that post-FMT changes in patient stool butyrate levels correlate with an increase in similarity of patient and donor microbiomes.

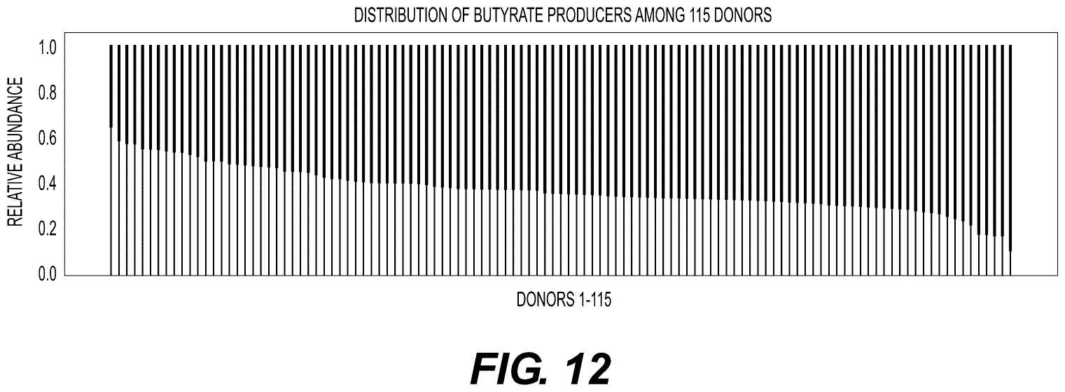

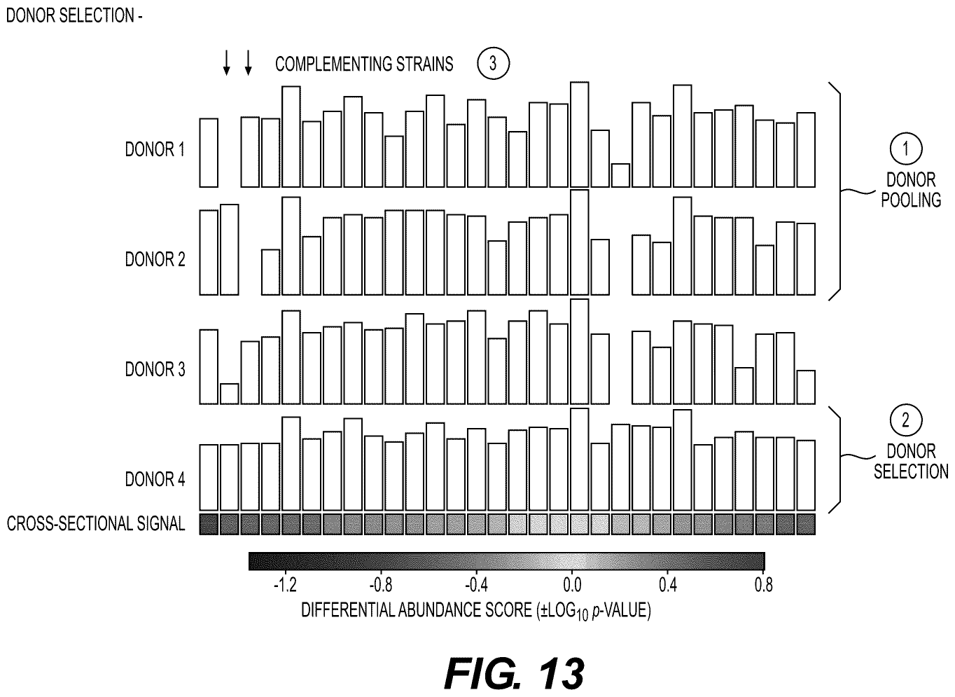

FIG. 12 shows that the relative abundance of SCFA-producing bacterial strains (i.e., butyrate producers) in stool of 115 stool donors varies across the donors. To generate the graph, 16S rRNA sequences of OTUs from stool of each donor were compared to 16S rRNA sequences from bacterial strains predicted to be butyrate-producers based on published data (Vital et al. (2017), “Colonic butyrate-producing communities in humans: an overview using omics data” American Society for Microbiology 2(6): e00130-17; Vital et al., (2014), “Revealing the bacterial butyrate synthesis pathways by analyzing (meta)genomic data” mbio 5(2): e00889-14). Relative abundance for each donor was determined by summing the number of reads from butyrate-producing OTUs in each sample, and dividing by the total number of bacterial reads obtained from the sample.

### Example 5: Selection of Donors for Treatment of Ulcerative Colitis

There have been reports of variable donor efficacy in indications such as ulcerative colitis (UC). Fecal transplants and other donor-derived therapies can vary widely in the abundances of microbial strains and species, and this could result in differences in drug potency. As illustrated in FIG. 1, three approaches can serve to minimize product variability: donor selection, pooling, and strain complementation. Donors or stools can be selected for the presence of desired characteristics. Pooling across donors can increase the chance that non-ubiquitous organisms are included in the product, although these will be diluted in abundance. If there are certain components that are critical to deliver, a hybrid strategy of complementing a whole-community product with separately-cultured single strains provides another therapeutic approach. The whole-community product also provides a background healthy ecology to optimize engraftment of the added strains.

Natural variation in individual donors' presence and abundance of disease-relevant microbes motivates strategies for improving product uniformity. Differential abundance of major bacterial taxa is calculated by comparing UC versus non-IBD controls in the HMP2 16S amplicon sequencing dataset (red: enriched in UC; blue: depleted in UC; abundances collapse at Family level) (Lloyd-Price, J., et al. (2019). “Multi-omics of the gut microbial ecosystem in inflammatory bowel diseases.” Nature 569, 655-662). Abundances of these taxa are shown in the four donors used in the Jacob (2017) FMT trial in UC (yellow bars) (Jacob et al., (2017). “Single delivery of high-diversity fecal microbiota preparation by colonoscopy is safe and effective in increasing microbial diversity in active ulcerative colitis.” Inflammatory Bowel Disease 23(6): 903-911). Two UC-depleted taxa that vary in prevalence across the donors are indicated (orange arrows). Three mitigation strategies are shown: (1) In Jacob et al., patients each receive two of the four donors; this donor pooling strategy increases the diversity of strains delivered. (2) Pre-screening for donors containing desirable components; for instance, Donor 1 contains all the identified UC-depleted taxa. (3) Complementation by co-administering separately-cultured strains in order to ensure uniform delivery of critical components along with the full community.

1. A pharmaceutical composition comprising a preparation of fecal bacteria from a stool of a healthy human donor, wherein the fecal bacteria are selected to produce at least one short chain fatty acid (SCFA), wherein the preparation of fecal bacteria comprises uncultured bacteria, and wherein the preparation of fecal bacteria comprises lyophilized bacteria.

2. A method of delivering a short chain fatty acid (SCFA) to an intestine of a subject in need thereof, the method comprising administering to the subject a pharmaceutical composition comprising a preparation of uncultured fecal bacteria derived from a stool of a healthy human donor, wherein:
the uncultured fecal bacteria are selected to produce the SCFA at or above a threshold level,
fecal bacteria in a stool of the healthy human donor produce the SCFA at or above a threshold level, or
the healthy human donor produces a stool comprising the SCFA at or above a threshold level.

3. A method comprising determining a level of at least one SCFA produced by fecal bacteria of a subject; and administering to the subject a pharmaceutical composition based on determining the level of the at least one SCFA to be below a threshold level, wherein the pharmaceutical composition comprises a preparation of uncultured fecal bacteria of stool of a healthy human donor.

4.-41. (canceled)
